(19) United States
(12) Patent Application Publication
(10) Pub. No.: US 2006/0210536 A1
(43) Pub. Date: Sep. 21, 2006

(54) Methods and compositions for inhibiting stat signaling pathways

(76) Inventors: Curt Horvath, Wilmette, IL (US); Jason Rodriguez, Evanston, IL (US); Christina Marie Ulane, New York, NY (US); Jean-Patrick Parisien, Wilmette, IL (US)

Correspondence Address: DORSEY & WHITNEY LLP;INTELLECTUAL PROPERTY DEPARTMENT, 250 PARK AVENUE, NEW YORK, NY, 10177, US

(21) Appl. No.: 10/553,160

(22) PCT Filed: Apr. 19, 2004
(86) PCT No.: PCT/US04/12066

§ 371 Date: Mar. 6, 2006

Related U.S. Application Data
(60) Provisional application No. 60/463,764, filed on Apr. 17, 2003.

(51) Int. Cl. A61K 48/00; C12N 15/867

(52) U.S. Cl. 424/93.2; 435/456

(57) ABSTRACT

The invention relates to compositions and methods for modulating cell signaling mediated by signal transducers and activators of transcription (STAT). The compositions target cellular STAT3 and STAT1 protein, particularly STAT3, for degradation via the ubiquitination pathway. Thus, the STAT inhibiting agents are useful for inhibiting STAT mediated signal transduction events, such as responses to IL6 and v-Src, any may be applied to treating diseases associated with activated STAT proteins, particularly STAT3 activity, such as cell proliferative disorders, inflammatory reactions, and autoimmune conditions.

### CROSS REFERENCES TO RELATED APPLICATIONS

This application claims the benefit of U.S. Provisional Application No. 60/463,764, filed Apr. 17, 2003, which is incorporate herein by reference.

### STATEMENT OF GOVERNMENTAL INTEREST

This invention was made in the course of research sponsored by the National Institutes of Health under Grant No. R101AI507707-01A1. The Government may have certain rights in the invention.

### TECHNICAL FIELD

The present Invention relates generally to compositions and methods for inhibiting cellular signaling pathways, particularly for decreasing STAT signaling activity. Further provided are methods of using the compositions for inhibiting cancer cell growth and modulating immune responses related to inflammatory reactions, particularly inflammatory disorders and autoimmune diseases.

### BACKGROUND OF THE INVENTION

Cytokines and growth factors act by binding to their cognate receptors on the cell surface and Initiating signal transduction events that ultimately lead to cellular responses to the extracellular stimuli. The family of signal transducer and activator of transcription (STATs) proteins are important mediators of the intracellular signaling pathways initiated by certain cytokines and growth factors. STATs are latent transcription factors, which become phosphorylated after recruitment to ligand-activated receptors. The activated STATs dimerize and translocate to the nucleus where they regulate expression of various target genes. Seven STATs (STAT1-6, with two STAT5 genes) have been identified in mammalian cells and are implicated in controlling cell growth, cell differentiation, organogenesis, embryonic development, and host responses to cancer and infection. Homologs of STAT proteins present in other eukaryotes, such as Dictyostelium, Caenorhabditis elegans, and Drosophila, also appear to regulate various developmental processes in these organisms (Kisseleva, T. et al., Gene 285:1-24 (2002)).

STAT proteins display a number of conserved features. The amino terminal part of the protein participates in cooperative interactions involved in DNA binding. An adjacent coiled-coil domain functions as an interaction region for a number of regulatory modifiers, including interferon regulatory factor IRF-9 and STAT-interacting protein StlPI (Collum, R. G. et al., Proc. Natl. Acad. Sci. USA 97(18):10120-10125 (2000)). The DNA binding domain at the carboxy terminal half of STAT recognizes GAS type enhancer sequences on target genes while transcriptional activation occurs through the transcriptional activation domain (TAD) localized toward the carboxy terminus. The length and sequence of the TAD vary substantially between STATs. Src homology 2 domain (SH2) is the most conserved region of STATs and mediates recruitment of STAT to receptors and STAT dimerization. A linker region separating the SH2 domain from the DNA binding region appears to function as a buffer to limit perturbations to DNA binding interactions.

Upon receptor activation by ligand binding, the receptor tyrosine kinase or receptor-associated family of cytoplasmic kinases, such as Janus kinase (JAK), Tyk2, and src kinases, phosphorylates the receptor to generate docking sites for the SH2 domains of STAT. Following recruitment, the receptor bound STATs become phosphorylated at the SH2 domains, dimerize, and then translocate to the nucleus where they bind to GAS related sequences in the control regions of target genes, thereby regulating their transcription. Activated STATs are found as homodimers, and in some circumstances as heterodimers, depending on the extracellular ligand. For instance, IFNα signaling involves a dimer of STAT 1 and STAT2; IL6 signaling involves a dimer of STAT1 and STAT3; and growth hormone signaling involves a dimer of STAT5a and STAT5b.

Of the known mammalian STAT proteins, STAT1, STAT2, and STAT3 display the broadest expression profiles and regulate cellular responses to extracellular ligands in most cell types (Zhong, Y. et al., Oncogene 21:217-226 (2002); Zhong, Z. et al., Proc. Natl Acad. Sci. USA 91:4806-4810 (1994)). Activated STAT1 and STAT2, acting in a protein complex (e.g., ISGF3) with proteins IRFp48, ISGF3γ, and IRFp9, mediate induction of type I interferon (IFNα/β) target genes. Similarly, STAT1 homodimer mediates transcriptional responses to type II interferon (e.g., IFNγ), which is necessary for development of innate and adaptive immune responses (Bach, E. A. et al., Ann. Rev. Immunol. 15:563-591 (1997); Ikeda, H. et al., Cytokine Growth Factor Rev. 13:95-109)). Confirming the physiological role of STAT1, inactivation of the STAT1 gene in mice results in animals with defective immune responses as a result of disruptions to IFN signaling.

STAT3 is activated by a variety of cytokines including, among others, interleukin-6 (IL6), leukemia inhibitory factor (LIF), ciliary neurotrophic factor (CNTF), oncostatin M, and leptin (Hirano, T. et al., Oncogene 19:2548-2556 (2000)). STAT3 is also activated by growth factor receptors with intrinsic protein tyrosine kinase activity, Including, among others, PDGF, EGF, G-CSF, and Neuregulin-1 (NRG-1) receptors (Sadowski, H B. et al., Science 261:1739-1744 (1993); Vignais, M. L. et al., Mol. Cell Biol. 16:1759-1769 (1996); Liu, J. and Kern, J. A., Am. J. Respir. Cell Mol. Biol. 27(3):306-313 (2002)) as well as cellular and viral cytoplasmic tyrosine kinases, such as c-Src and v-Src (Bromberg, J. et al., Mol. Cell Biol. 18:2553-2558 (1998), Yu, C. L., et al., Science 269:81-83 (1995)). STAT3 plays a role in cellular growth and differentiation of mammary gland, liver, keratinocytes, thymus, blood, and neurons, and its activation is associated with the inflammatory process, liver regeneration, acute phase responses, and other physiological responses (Akira, S., Oncogene 19:2607-2611 (2000); Levy, D. E. and Lee, C. K., J. Clin. Invest. 109:1143-1148 (2002)). In concert with the role of STAT3 in cytokine and growth factor signaling, targeted disruption of the STAT3 gene in mice results in early embryonic lethality (Takeda, K. et al., Proc. Natl. Acad. Sci USA 94:3801-3804 (1997)). STAT3 is frequently found hyper-activated in human cancers and cancer cell lines, and studies implicate STAT3 as an oncogene involved in malignant transformation ((Darnell, J. E., Nat. Rev. Cancer 2:740-749 (2002), Bromberg, J. et al., supra; Yoshida, T. et al., J. Exp. Med. 196:641-653 (2002); and Yu, C. et al., supra) and tumor maintenance, as suggested by spontaneous programmed cell death following STAT3 inhibition in cancer cells (Catlet-Falcone, R. et al., Immunity 10:105-115 (1999), Grandis, J. R. et al., Proc. Natl. Acad. Sci. USA 97:4227-4232 (2000); Niu, G. et al., Cancer Res. 61:3276-3280 (2001); Niu, G. et al., Oncogene 21:2000-2008 (2002)). Other disorders associated with constitutive STAT3 activity Include rheumatoid arthritis, asthma, and HIV infection (Turkson, J. and Jove, R., Oncogene 19:6613-6626 (2000)). The significant role of STAT3 in normal and disease processes suggests the desirability of identifying inhibitors specifically directed to STAT3 (Buettner, R. et al., Clin. Cancer Res. 8:945-954 (2002); Darnel, J. E., supra).

Modulating STAT activity may be accomplished by targeting any number of STAT properties, including (a) recruitment to the receptor, (b) phosphorylation of SH2 domains, (c) STAT dephosphorylation (d) STAT dimerization, (e) nuclear translocation, (0 DNA binding and transcriptional activition, (g) STAT protein expression, and (h) intracellular half-life of STAT.

A number of STAT modulators are described in the art. Peptide agents affecting recruitment are described in U.S. Pat. No. 5,731,155. These peptides or peptidomimetics are receptor sequences phosphorylated by the intrinsic receptor tyrosine kinase or the associated cytoplasmic kinases, and thus mimic receptor domains Interacting with STAT. Binding of these peptides to STAT may prevent receptor recruitment of STATs. Kinase inhibitors, including, among others, piceatannol (JAK1), tyrphostin AG490 (JAK2), and parthenolide (JAK2), are described in Su, L. and David, M., J. Biol. Chem. 275:12661-12666 (2000); Meydan, N. et al., Nature 379:645-648 (1996); and Sobota, R. et al., Biochem. Biophys. Res. Commun. 267:329-333 (2000)). Phosphatases that attenuate STAT activity by dephosporylation is described in Aoki, N. and Matsuda, T., J. Biol. Chem. 275:39718-39726 (2000) and Wu, T. R. et al., J. Biol. Chem. 277:47572-47580 (2002)). Disruption of transcriptional activation may be accomplished through use of dominant negative forms of STAT. For example, STAT3β isoform lacking the transcriptional activation domain appears to dimerize with STAT3 and prevent transcription of respective target genes (Niu, supra). Inhibition of STAT protein expression with antisense oligonucleotides is described in Grandis, J. R. et al., J. Clin. Invest. 102:1385-1392 3(1998)).

Decreasing the half-life of STAT provides another basis of modulating STAT activity. It is known that infection of cells with certain negative-strand RNA viruses (e.g., Paramyxovirinae from the genus Rubulavirus) decreases the stability of STAT1 and STAT2 (Didcock, L. et al., J. Virol. 73:9928-9933 (1999); Goodbourn, S. et al., Gen. Virol. 81:2341-2364 (2000); Parisien, J. P. et al., Virology 283:230-239 (2001), Parisien, J. P. et al., J. Virol. 76:4190-4198 (2002); Parisien, J. P. et al., J. Virol. 76:6435-6441 (2002), Yokosowa, N. S. et al., J. Virol. 76:12683-12690 (2002); Young, D. F. et al., Virology 269:383-390 (2000)). This property is attributable to a viral gene product, the V protein, responsible for assembling a degradation complex (VDC) containing V protein itself, both STAT1 and STAT2, and V-interacting cellular proteins that include the damaged-DNA binding protein, DDB1 (Andrejeva, J. E. et al., J. Virol. 76:11379-11386 (2002), Lin, G. Y. et al., Virology 249:189-200 (1998); and Ulane, C. M. et al., Virology 304:160-166 (2002)), and the cullin family member Cul4A (Ulane, C. M. et al., supra). This protein complex functions as a STAT-specific E3 ubiquitin ligase enzyme, efficiently inducing STAT1 and STAT2 polyubiquitination and proteasomal degradation. By destabilizing STAT1 in the cell, the virus attenuates the anti-viral consequences of IFN signaling, thereby evading a key host defense mechanism. The STAT1 destabilizing properties of V protein is best described for simian virus 5 (SV5) and type II human parainfluenza virus (HPIV2). Infection with SV5 or expression of SV5 V protein targets STAT1 for ubiquitin-mediated proteasome degradation. Similarly, infection with HPIV2 or expression of the HPIV2 V protein targets STAT2 (Didcock, L. et al., supra; Goodbourn, S. et al., supra; Kubota, supra, Parisien, J. P. (2002), supra).

The Mumps V protein has about 42% amino acid identity with SV5 and about 37% identity with HPIV2. Mumps virus V protein is known to mediate STAT1 and STAT2 degradation (Yokosawa, N. S. et al., J. Virol. 76:12683-12690 (2002); Kubota, T. et al., Biochem. Biophys. Res. Commun. 283:255-259 (2001); Fuji, N. et al., Virus Res. 65:175-185 (1999)). The carboxyl terminal region of Mumps V protein interacts with STAT proteins in vitro (Nishio, M. D. et al., Virology 300:92099 (2002), although it is not necessary for ubiquitin-mediated degradation of STAT1 (Yokosawa, N. S. et al., supra). Intact Mumps virus has been used to treat malignant cells, albeit based on its ability to induce cell death upon infection (Nemuniaitis, J., Investigational New Drugs 17:375-386 (1999)). The oncolytic property of Mumps virus was attributable to an unknown factor present in the media of cells following viral infection. (Miki, S. et al., FEBS 278:179-182 (1991)).

Since the art describes agents capable of affecting stability of STAT1 and its potential use for regulating STAT1 activity, it is desirable to Identify agents that specifically alter stability of other STATs, particularly STAT3 given its broad role in mediating cellular responses to a variety of cytokines and growth factors in many cell types. Targeting specific STATs by affecting stability is advantageous because of the irreversible nature of the inhibition and absence of side effects arising from inhibition of other signaling pathways. These agents will have particularly useful applications in treating diseases related to tumorigenesis and immune dysregulation.

### SUMMARY OF THE INVENTION

In summary, the present invention provides for a method of inhibiting STAT activity in a sample, comprising adding to the sample a sufficient amount of a STAT inhibiting agent capable of forming a complex with STAT protein and inducing a decrease in levels of said STAT protein. Preferably, the STAT protein is STAT3 or STAT1, more preferably STAT3.

Also provided herein Is a method of inhibiting STAT mediated signaling in a cell, comprising contacting the cell in vivo, in vitro, or ex vivo with a STAT inhibiting agent capable of forming a complex with STAT protein and inducing a decrease in levels of the STAT protein, preferably STAT1 or STAT3, more preferably STAT3. The cell can be any cell expressing STAT proteins, particularly cells with elevated levels of STAT3 activity. These include, among others, tumor cells or lymphocytes (e.g., T cell) displaying STAT3 activity.

In one aspect, the STAT inhibiting agent comprises a protein or peptide having at least about 80% identity to the amino acid sequence of FIG. 1 (SEQ ID NO:1). More preferably, the inhibiting agent comprises a peptide of SEQ ID NO: 1, or a fusion protein or a peptide fragment thereof. In one embodiment, the present invention provides for use of a substantially pure preparation of the STAT inhibiting agent.

In another aspect, the STAT inhibiting protein or peptide is encoded by a nucleic acid. In one embodiment, the nuclei acid has at least about 80% identity to the nucleic acid sequence of FIG. 2 (SEQ ID NO:2). More preferably, the nucleic acid has the nucleic acid sequence of SEQ ID NO:2. The nucleic acid is an isolated nucleic acid, or a recombinant nucleic acid which encodes the STAT inhibiting protein or peptide.

In a further embodiment, the nucleic acids are part of expression vectors used for expressing the STAT inhibiting agents in cells. These include vectors for expression in various cell types, including yeast, insect, avian, and mammalian cells. One embodiment of the expression vectors are viral vectors for delivery into cells. These include retroviruses, lentiviruses, adenoviruses, adenoassociated viruses, alphaviruses, and the like, which can be used to express the STAT inhibiting protein when administered to a mammal.

The STAT inhibiting agents are applicable in a variety of contexts for regulating STAT activity. In one aspect, the present invention provides for a method of inhibiting growth of a tumor cell, where the method comprises contacting the tumor cell with a composition comprising a STAT inhibiting agent in an amount sufficient to inhibit tumor cell growth, particularly in those tumors where abnormal activity of STAT, particularly the activities of STAT1 or STAT3 or variants thereof, is correlated with formation or maintenance of the tumor. The STAT inhibiting proteins are particularly applicable to tumor cells in which elevated levels or constitutive expression of STAT3 is associated with the tumor cell growth. Exemplary tumor cells include, among others, multiple myeloma; leukemia, including HTLV-1 dependent, chronic lymphocytic leukemia, erythroleukemia, acute myelogenous leukemia, acute lymphocytic leukemia, large granular lymphocyte leukemia; lymphomas, including EBV related Burkitt's, mycosis fungoides; HSV saimiri-dependent (T-cell); cutaneous T-cell lymphoma, Hodgkins disease; and solid tumors, including breast cancer, SCCHN, renal cell carcinoma, melanoma, ovarian carcinoma, lung cancer, prostate carcinoma, and pancreatic adenocarcinoma.

The STAT inhibiting agents may be used alone, or used in combination with other therapeutic agents, preferably chemotherapeutic agents, such as DNA damaging agents, mitotic spindle inhibitors, STAT phosphorylation inhibitors, or STAT dimerization inhibitors (e.g., STAT dominant negative mutants).

The present invention further provides for a method of inhibiting an inflammatory reaction, where the method comprises administering to a subject in need thereof an effective amount of a STAT inhibiting agent, whereby the inflammatory reaction is inhibited. In this embodiment, the inflammatory reaction is associated with abnormal STAT activity, particularly the activities of STAT3 or STAT1 or variants thereof. The method is particularly applicable to inflammatory reactions associated with elevated or constitutive levels of STAT3 activity. Exemplary diseases which may be treated with the subject agents include, e.g., Crohn's disease, inflammatory bowel disease, multiple sclerosis, ischemia, stroke, traumatic brain injury, spinal injury, rheumatoid arthritis, atherosclerosis, and Alzheimer's disease.

In another aspect, the present invention provides for methods of treating an autoimmune disease, comprising administering to a subject an effective amount of a STAT inhibiting agent which forms a complex with STAT protein and decreases levels of said STAT protein, thereby by ameliorating the autoimmune disease. Similar to other conditions described herein, the autoimmune disease is associated with abnormal STAT activity, particularly the activities of STAT1 or STAT3 or variants thereof. The methods are particularly directed to autoimmune conditions associated with elevated or constitutive levels of STAT3 activity. Exemplary autoimmune diseases include insulin dependent diabetes mellitus, systemic lupus erythematosus, and psoriasis.

In yet a further aspect, the present invention provides a method of inhibiting signaling by compositions that exert their biological effects through the STAT signalling pathway. These include growth factors and cytokines, such as IFNs, EGF, IL5, IL6, IL-10, HGF, LIF, or BMP2. In particular, the method is useful in specifically inhibiting action of factors acting through the STAT3 mediated signalling pathway.

### BRIEF DESCRIPTION OF THE DRAWINGS

FIG. 1 shows the amino acid sequence of Mumps V protein (SEQ ID NO: 1).

FIG. 2 shows the nucleotide sequence of the Mumps V protein cDNA sequence beginning with the initiating methionine (SEQ ID NO:2). Lower case refers to sites where nucleotide differs from GenBank: Position 216: AGC(Ser) instead of AGA(Arg); Position 382: GCG(Ala) instead of ACG(Thr); Position 434: ACC(Thr) instead of ATC(Ile).

FIG. 3 shows the amino acid sequence of human STAT-3 protein, isoform 1 (Accession No.: GI: 21618340/NP 644805)

FIGS. 4A and 4B show the nucleotide sequence of human STAT-3 protein (Accession No.: GI: 21618339/NM 139276.1). The coding region spans nucleotide residues 241-2553. The initiating methionine and the termination codons are underlined.

FIG. 5 shows the amino acid sequence of human STAT-1 protein, isoform-α (Accession No.: GI: 6274552/NM 009330.1).

FIGS. 6A and 6B show the nucleotide sequence of human STAT-1 protein (Accession No.: GI: 21536299/NM 007315.2.1). The coding region spans nucleotide residues 352-2604. The initiating methionine and termination codons are underlined.

FIG. 7A shows blocking of IFN signal transduction by Mumps virus V protein. 293T cells were transfected with an ISRE-luciferase reporter gene (left panel) and either empty vector or Mumps V protein expression vector as indicated. Cells were treated with (IFN-α) or without (UNT) 1000 U/ml IFNα for 6 hours prior to lysis and luciferase assays. ISRE is an enhancer sequence bound by ISGF3 complex. The same experiment was carried out using a GAS-luciferase reporter gene (right panel) and treatment with or without 5 ng/ml IFNγ. All bars represent average values from triplicate samples, normalized to co-transfected CMV-lacZ, ±standard deviation. FIG. 7B shows targeting of both STAT1 and STAT3 by Mumps virus V protein. 2fTGH cells were transfected with empty vector or FLAG-tagged Mumps V expression plasmid and subjected to indirect immunofluorescence staining 24 h later. Cells were fixed, permeabilized, and stained sequentially for FLAG, then STAT1 or STAT2, and analyzed by confocal microscopy. Nuclei were visualized by staining with TOTO3. Arrows point to the location of V-expressing cells. FIG. 7C shows that Mumps V protein interferes with cytokine responses. Cells were treated similar to panel 7B, except that IFNα, IFNγ, or IL6 was added 30 minutes prior to fixation. FIG. 7D shows effect of Mumps virus infection on STAT degradation and relocalization. Cells were infected with Mumps virus (2 pfu/cell) and at 20 h post infection, processed for indirect immunofluorescence as described above, except that an antibody specific for Mumps virus nucleocapsid protein (NP) was used to detect infected cells.

FIGS. 8A-8E show that STAT3 interference prevents IL6 and v-Src transcription responses. Specifically, FIG. 8A shows Mumps V inhibition of IL6 signaling. GAS-luciferase reporter gene assays were carried out in 2fTGH cells as in FIGS. 7A-7D, but cells were transfected with or without SV5 V or Mumps V as indicated, then treated without (UNT) or with IL6 (400 ng/ml) plus soluble IL6 receptor (500 ng/ml) as described in Guschin, D. et al., EMBO J. 14:1421-1429 (1995). FIG. 8B shows Mumps V inhibition of v-Src signaling. GAS-luciferase was assay carried out as in FIG. 8A, but with co-transfected v-Src expression vector as the STAT3 activator. FIGS. 8C-8E show somatic cell genetic analysis of Mumps V interference. Specifically, FIG. 8C demonstrates that IL6 evasion does not require STAT1. IL6 transcription assay carried out as in FIG. 8A, but using STAT1-deficient U3A cells. FIG. 8D shows that IFNγ evasion requires STAT2. IFNγ response assay was carried out as in FIGS. 7A-7D in the absence or presence of co-transfected human STAT2 expression vector. FIG. 8E shows that IL6 evasion does not require STAT2. IL6 response assay was carried out as in FIG. 8A, but using STAT2-deficient U6A cells.

FIGS. 9A-9B show Mumps V interference of STATs operates in murine cells. Specifically, in FIG. 9A, Mumps V blocks IFNβ signaling in NIH 3T3 cells. ISRE-luciferase reporter gene assay was carried out in murine NIH 3T3 fibroblasts as in FIGS. 7A-7D, but the cells were stimulated with 500 U/ml murine IFNβ as indicated in the presence or absence of co-transfected human STAT2 expression vector. FIG. 9B shows blockade of v-Src signaling in murine cells by V protein. GAS-luciferase assay was carried out in NIH 3T3 cells or an NIH 3T3 cell line expressing human STAT2 as indicated.

FIGS. 10A-10C show affinity purification of a Mumps V protein-dependent complex. FIG. 10A shows eluate from FLAG affinity purification separated on acrylamide gels and silver stained. Electrophoretic mobilities of prestained molecular weight markers (M) and general V-interacting protein (VIP) subunits or Mumps V-specific interacting protein (MuVIP) subunits are indicated by their apparent molecular weight in kDa. FLAG-green fluorescent protein (GFP), FLAG-SV5 V (SV), or FLAG-Mumps V (MuV) is indicated. FIG. 10B shows identification of VIP subunits. Parallel samples from experiment in FIG. 10A were processed for immunoblotting with antisera for DDB1, STAT1, STAT2, STAT3, or CuI4A. FIG. 10C shows that V protein induces STAT1 and STAT3 polyubiquitylation. Transfected cell extracts were immunoprecipitated (IP) with STAT1 (top) or STAT3 (bottom) antisera, then processed for immunoblot (IB) with antiserum for ubiquitin (Ub; left panels). Filters were re-probed for the precipitated STAT (right panels).

FIG. 11 shows functional expression of Mumps V protein from recombinant lentivirus vectors and effect on STAT levels. Human fibrosarcoma cells infected with recombinant virus were processed by preparing cell lysates. Lysates were then separated by electrophoresis, immunoblotted, and processed with antiserum to STAT1, STAT2, and STAT3. The SV5 V protein expression reduces STAT1 steady state levels, HPIV2 V protein reduces STAT2 levels, and Mumps V protein reduces both STAT1 and STAT3 levels. Recombinant lentiviruses express: GFP, green fluorescent protein; SV, SV5 V protein; N100D, variant of SV5 V protein; HV, HPIV2 V protein; MuV; Mumps virus V protein; and MeV measles virus V protein. MI is mock infected, cells. Positions of molecular mass markers indicated on left.

### DETAILED DESCRIPTION OF THE INVENTION

The present invention relates to compositions and methods for attenuating or inhibiting the activity of signal transduction and activators of transcription (STAT) family of proteins, in particular for inhibiting the activity of STAT3 and/or STAT1 related proteins. Generally, the STAT family of proteins regulates the responses of a cell to various cytokines and peptide growth factors. STAT-3 is activated by cytokines such as interleukin-6 (IL6), leukemia inhibitory factor (LIF), ciliary neurotrophic factor (CNTF), oncostatin M, and leptin. Growth factors, for example PDGF and EGF, also mediate their effects via STAT-3 signal transduction. Upon activation, STAT-3 regulates expression of various genes involved in apoptosis and cell growth. Constitutive or elevated STAT-3 activity is associated with certain human cancers and cancer cell lines. STAT1 mediates cellular responses to type I and type II interferons, and plays viral role in development of adaptive and innate immune responses.

Given the role of STAT1 and STAT-3 proteins in modulating the responses of cells to cytokines and growth factors, the present invention finds use in inhibiting STAT1 and/or STAT3 protein, particularly STAT3 protein, in a variety of contexts. More specifically, the compositions and methods provided herein will find particular applications in inhibiting STAT mediated signaling, inhibiting the growth of cancer cells, and for modulating immune responses, including inflammatory disorders and autoimmune diseases.

Accordingly, the present invention provides for agents which inhibit STAT protein activity, especially STAT-1 and/or STAT-3, and in particular the activity of STAT3. The “STAT inhibitors” or “STAT inhibiting agents” disclosed herein have the one or more of the following characteristics, including, but not limited to 1) forming a complex with a STAT protein, 2) decreasing levels of STAT protein activity, particularly by inducing degradation of STAT protein, 3) increasing the ubiquitinylation of STAT protein, 4) attenuating or inhibiting expression of target genes activated by STAT, especially target genes regulated by STAT 3 or STAT 1, and 5) inhibiting activity of cytokines, particularly IL6, LIF, CNTF, oncostatin, and leptin; and growth factors, such as PDGF, EGF, and NRG-1, as further described below.

An inhibiting agent capable of “forming a complex with a STAT protein” refers to a “STAT complexing agent” that is specifically associated with STAT protein under conditions effective to promote complex formation, particularly under conditions approximating physiological conditions. The association may be direct in that the inhibiting agent physically interacts with STAT, or indirect in that the association is via other components of the complex, for example other proteins. Specific association as opposed to nonspecific association can be ascertained by various methods known in the art. Complex formation may be determined by immunoprecipitation with antibodies directed to any component of the complex, such as STAT 3 protein or the STAT inhibiting agent itself. Protein complexes stable to precipitation by antibodies are considered specific complexes comprising a STAT protein and inhibiting agent. In addition, inclusion of a non-STAT interacting agent in the immumoprecipitation assays provides a measure of the specificity of interaction of the STAT inhibiting agent. Similarly, specific associations are also assayable by affinity columns or beads, for example use of bound STAT protein or STAT inhibiting agent; by separation on solid phases, including, chromatography of complexes in sieving media; or ultracentrifugation, for example velocity sedimentation or equilibrium sedimentation. Alternatively, the assay for specific association is determined in vivo, for instance by using a two or three hybrid screens (Golemis, E. A. et al., in Current Protocols in Molecular Biology (Ausubel, F. M., ed), pp. 20.1.1-20.1.35, John Wiley & Sons, Inc., New York (1997); Licitra, E. J. and Liu, J. O., Proc. Natl. Acad. Sci. USA 93:12817-12821 (1996); Fields and Song, Nature 340:245-246 (1989); all publications incorporated by reference).

STAT inhibiting agents of the present invention also possess the activity of attenuating or suppressing the levels of STAT activity, particularly by decreasing the levels of STAT protein in a cell. By decreasing activity of STAT protein is a decrease in levels of STAT protein. This characteristic may be measured by immunoprecipitation, Western blotting, immunofluorescence, reporter tagged STAT protein (e.g., green fluorescent tagged STAT), and the like. By attenuating or decreasing STAT activity or STAT protein herein is meant that the STAT inhibiting agents reduces, decreases, lowers, or inhibits levels of STAT activity in the cell or cell free extract as compared to activity in the absence of inhibiting agent.

Another property associated with the STAT inhibiting agents is their capability to induce or enhance ubiquitinylation of STAT protein. Ubiquitinylation is the covalent attachment (e.g., conjugation) of ubiquitin moieties, a peptide of about 76 amino acids, to a target protein or protein substrate by the ubiqutin pathway, generally comprised of ubiquitin activating enzyme, ubiquitin conjugating enzyme, and ubiquitin ligase (see Hershko, A. et al., Nat Med. 6:1073-1081 (2000)). Tagging of the target protein generally targets the protein for destruction by the proteasome complex (Voges, D. et al., Ann. Rev. Biochem. 68:1015-1068 (1999)). STAT proteins may be modified by conjugation of mono-ubiquitin or by conjugation of multiubiquitin (polyubiquitin) moieties, preferably by polyubiquitin moieties, all of which are readily detected by ubiquitin specific antibodies or by use of labeled ubiquitin peptides. Inducing or enhancing ubiquitinylation is an increase in the amount or level of ubiquitin conjugated to STAT proteins as compared to levels of ubiquitinylation in the absence of STAT inhibiting agent.

STAT inhibiting agents exhibit the ability to inhibit expression of target genes normally activated by presence of STAT protein. For STAT3, these include, but are not limited to, target genes BCL-xl; Mcl-1, c-myc; and cyclin D1. Expression of these genes are readily detected by operably linking the nucleic acid control elements responsible for STAT dependent transcription activation to reporter or selection genes, such as genes encoding green fluorescent protein, β-galactosidase, chloramphenicol acetyl transferase, and the like. Alternatively, the proteins encoded by the target genes are detected directly, for example by use of gene product specific antibodies.

STAT inhibiting agents also inhibit cell signaling by various cytokines and growth factors. In the present invention, cytokines and growth factors acting through STAT-3 or STAT-1 are inhibited. One example is IL6, which is involved in increasing antibody production by inducing secretion of antibody from plasma cells following proliferative signals; inducing acute phase proteins; costimulation of T cells; and promoting generation of cytotoxic lymphocytes. IL6 has been described as both a pro-inflammatory and anti-inflammatory molecule, a modulator of bone resorption, a promoter of hematopoiesis, and an inducer of plasma cell development. IL6 also has been shown to influence IL4 production. It has been suggested that IL6 may influence naive CD4+ T cells to produce IL4 and express the IL4 receptor, and consequently, direct their own phenotype commitment. As indicated above, other extracellular ligands operating through STAT3 include as non-limiting examples cytokines LIF, CNTF, oncostatin, and leptin; and growth factors PDGF, EGF, G-CSF and NRG-1.

In a one embodiment, the STAT inhibitory agents comprise proteins related to members of viral V structural proteins found in negative-strand viruses, preferably the V protein of Paramyxoviruses, and particularly Mumps virus V protein. Protein in this sense includes proteins, polypeptides, and peptides.

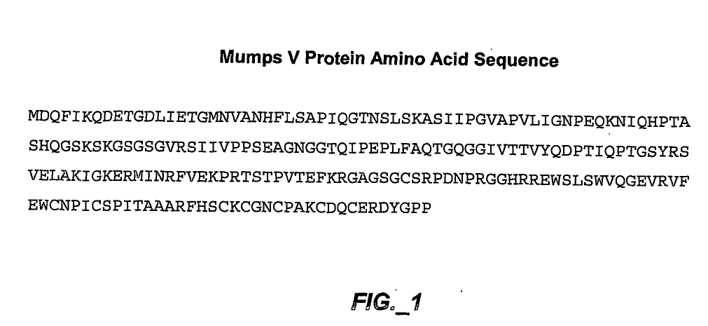

Accordingly, by “STAT inhibitor protein” or “STAT inhibiting agent” or “inhibiting agent” herein is meant a protein or peptide having significant homology to the amino acid sequence of FIG. 1 (SEQ ID NO:1) and characterized by the properties defined herein for a STAT inhibiting agent. These include mutants and natural variants of amino acid sequence of FIG. 1 (SEQ ID NO:1) and others described herein. Thus, in one embodiment, the STAT inhibiting agent is an isolated or purified protein or peptide of the amino acid sequence of FIG. 1 (SEQ ID NO:1), and it variants.

As further defined below, a peptide is isolated or purified when it is substantially free of cellular materials or free of chemical precursors or other materials. The level of purification will be defined according to use, but the preparation allows for the desired function of the protein even in the presence of considerable amount of other compounds.

In another embodiment, the STAT inhibiting protein is encoded by an isolated or recombinant nucleic acid. An isolated nucleic acid is a nucleic acid, e.g., RNA or DNA, or mixed polymer, which is separated from other components which naturally accompany the native sequence, including flanking nucleic acid sequences and host components. Thus, the term encompasses a nucleic acid sequence which has been removed from its naturally occurring environment, and includes recombinant and cloned nucleic acids isolates, chemically synthesized nucleic acids, and analogs thereof.

As used herein, a “recombinant nucleic acid” refers to a nucleic acid initially formed in vitro, generally by the manipulation of the nucleic acid by polymerases, endonucleases, and ligases, in a form not found in nature. For example, an isolated nucleic acid or an expression formed in vitro by ligating nucleic acid molecules that are not normally joined, are considered recombinant molecules. It is to be understood that a recombinant nucleic acid introduced into a suitable host cell or organism may replicate, generally by using the in vivo cellular machinery of the host cells rather than the in vitro manipulations. Such nucleic acids, although replicated non-recombinantly are still considered recombinant for the purposes of the invention.

Various embodiments of Mumps virus V protein and nucleic acids are known In the art, and include the following sequences: Genbank Acc. No. D00663.1, GI:222149, cds: 1251-1925; Genbank Acc. No. D86175.1, GI:1468936, cds: 108-782; and Genbank Acc. Nos. AF314560.1, GI:14325906, cds: 1979-2653; AF314559.1, GI:14325896, cds: 1979-2653; AF314558.1, GI:14325886, cds: 1979-2653; AB000388.1, GI:1783175, cds: 1979-2653; AB000387.1, GI:1783174, cds: 1979-2653; AB000386.1, GI:1783173, cds: 1979-2653; AF280799.1, GI:11545407, cds: 1979-2653; AF467767.1, GI:18643326, cds: 1979-2653; AF338106.1, GI:15077508, cds: 1979-2653; AF201473.1, GI:7861760, cds: 1979-2653; AF345290.1, GI:19070168, cds: 1979-2653; AB040874.1, GI:7592770, cds: 1979-2653; AF314562.1, GI:14325926, cds: 1979-2653; and AF314561.1, GI:14325916, cds: 1979-2653 (“cds” denotes protein coding sequence regions and numbers refer to nucleotide residues).

Homology can be based upon the overall protein amino acid sequence or a nucleic acid sequence encoding the subject protein. As used herein, a protein is a STAT inhibiting agent if the overall homology of the protein sequence to the respective amino acid sequences shown in FIG. 1 (SEQ ID NO:1) is at least about 70%, preferably at least about 80%, more preferably at least about 85%, and most preferably at least about 90%. In some embodiments, the homology will be as high as about 93 to 95 or 98%. Homologous amino acid sequence are also intended to include natural allelic variations in the respective protein sequence.

Homology in this context means sequence similarity or identity, with identity being preferred. This homology will be determined using standard techniques known in the art, including, but not limited to, the local homology algorithm of Smith and Waterman, Adv. Appl. Math. 2:482-289 (1981); by the homology alignment algorithm of Needleman and Wunsch, J. Mol. Biol. 48:443-453 (1970); by the search for similarity method of Pearson, W. R. and Lipman, D. J., Proc. Natl. Acad. Sci. USA 85:2444-2448 (1988); by computerized implementations of these algorithms (GAP, BESTFIT, FASTA, and TFASTA in the Wisconsin Genetics Software Package, Genetics Computer Group, 575 Science Drive, Madison, Wis.); or the Best Fit sequence program described by Devereux, J. et al., Nucleic Acids Res. 12:387-395 (1984), preferably using the default settings, or by inspection.

In one embodiment, similarity is calculated by FastDB based upon the following parameters: mismatch penalty of 1.0; gap size penalty of 0.33; and joining penalty of 30.0 (“Current methods in Comparison and Analysis”, in Macromolecule Sequencing and Synthesis: Selected methods and Applications, (Schlesinger, D. H. Ed.) pp. 127-149, Alan R. Liss, Inc. (1998). Another example of a useful algorithm is PILEUP. PILEUP creates a multiple sequence alignment from a group of related sequences using progressive, pairwise alignments. It can also plot the clustering relationships used to create the alignment. PILEUP uses a simplification of the progressive alignment method of Feng and Doolittle, J Mol. Evol. 35:351-360 (1987); which is similar to that described by Higgins and Sharp, CABIOS 5:151-153 (1989). Useful PILEUP parameters include a default gap weight of 3.00, a default gap length weight of 0.10, and weighted end gaps.

An additional example of a useful algorithm is the BLAST algorithm, described in Altschul, S. F. et al., J. Mol. Biol. 215: 403-410 (1990) and Karlin, et al., Proc. Natl. Acad. Sci. USA 90: 5873-5887 (1993). A particularly useful BLAST program is the WU-BLAST-2 program described in Altschul et al., Methods Enzymol. 266:460-480 (1996) (see world wide web at blast.wustl/edu/blast/README.html). WU-BLAST-2 uses several search parameters, most of which are set to the default values. The adjustable parameters are set with the following values: overlap span=1, overlap fraction=0.125, and word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence Identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

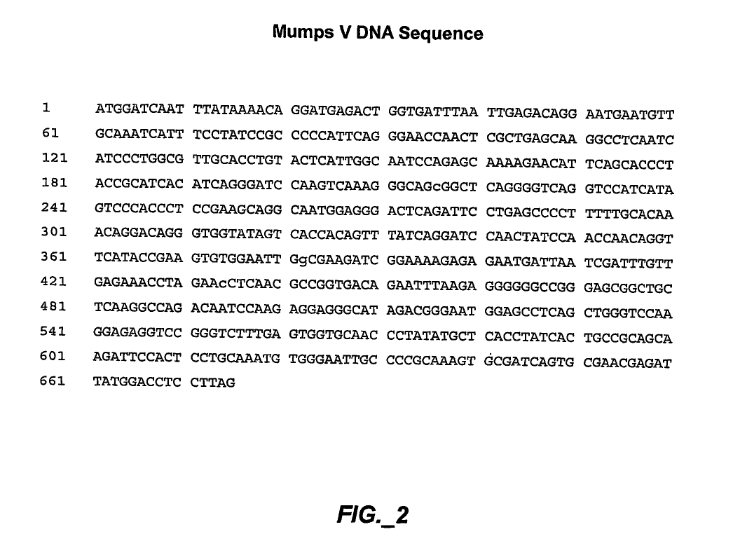

In a similar manner, “percent (%) nucleic acid sequence identity” with respect to the coding sequence of the polypeptides identified herein is defined as the percentage of nucleotide residues in a candidate sequence that are identical with the nucleotide residues in the coding sequence of the STAT inhibiting protein (FIG. 2) (SEQ ID NO:2). A preferred method utilizes the BLASTN module of WU-BLAST-2 set to the default parameters, with overlap span and overlap fraction set to 1 and 0.125, respectively. As with homology of amino acid sequences

An additional useful algorithm is Gapped BLAST as reported by Altschul, S. F. et al., Nucleic Acids Res. 25:3389-3402 (1997). Gapped BLAST uses BLOSUM-62 substitution scores; threshold T parameter set to 9; the two-hit method to trigger ungapped extensions; charges gap lengths of k a cost of 10+k; Xu set to 16; and Xg set to 40 for database search stage and to 67 for the output stage of the algorithms. Gapped alignments are triggered by a score corresponding to ˜22 bits.

In one embodiment, nucleic acids encoding the STAT inhibiting peptides will be encoded by nucleic acids having at least about 70% identity to the nucleic acid sequence of FIG. 2 (SEQ ID NO:2). Preferably, the nucleic acid will have at least about 80% sequence identity, more preferably at least about 90% sequence identity, and in a particularly preferred embodiment, a sequence identity of at least about 95% or more.

In addition, the alignment may include the introduction of gaps In the sequences to be aligned. In addition, for sequences which contain more or fewer amino acids than the protein sequences shown in FIG. 1 (SEQ ID NO:1), it is understood that the percentage of homology will be determined based on the number of homologous amino acids in relation to the total number of amino acids. Thus, for example, homology of sequences shorter than that shown in FIG. 1 (SEQ ID NO:1), as discussed below, will be determined using the number of amino acids in the shorter sequence.

Accordingly, in one embodiment, included within the definition of inhibitors herein are portions or fragments of the sequences provided herein. Fragments of STAT inhibitors are considered STAT inhibiting agents if a) they share at least one antigenic epitope; b) have at least the indicated homology; and c) have at one or more of the defined characteristics of STAT inhibiting agents, including, forming a complex containing a STAT protein; inducing a decrease in level of the STAT protein or activity, particularly through induced degradation; increasing ubiquitinylation of STAT protein; attenuating or inhibiting expression of target genes activated by STAT, particularly STAT 3 or STAT 1; and inhibiting activity of certain cytokines or growth factors, such as IL6, leukemia inhibitory factor (LIF), ciliary neurotrophic factor (CNTF), oncostatin M, and leptin. A useful fragment can be identified by routinely testing the fragments for the desired activity.

Thus, various deletion mutants of STAT inhibiting agents encompassed within the scope of the invention can be made. Particularly preferred are deletion mutants comprising the carboxy terminal cysteine-rich region comprising residues from about 170 to about 224.

In another embodiment, the present invention provides variants or muteins of STAT inhibiting agents. These variants fall into one or more of three classes: substitutional, insertional or deletional variants. These variants ordinarily are prepared by sited directed mutagenesis of nucleotides In the DNA encoding the inhibiting agent, for example with cassette, primer, or PCR mutagenesis or other techniques well known in the art, to produce DNA encoding the variant, and thereafter expressing the DNA in recombinant cell culture as outlined herein. However, variant proteins may be prepared by in vitro synthesis using established techniques. Amino acid sequence variants or muteins are characterized by the predetermined nature of the variation, a feature that sets them apart from naturally occurring allelic or interspecies variation in the amino acid sequences of the inhibiting agents. The variants or muteins typically exhibit the same qualitative biological activity as the naturally occurring analogue, although variants can also be selected which have modified characteristics, such as increased or decreased inhibiting activity. For example, the experimental results obtained by the inventors suggest that there may be separate STAT1 and STAT3 targeting complexes formed, and thus one embodiment comprises a variant Mumps V protein capable of forming only one such targeting complex.

While the site or region for introducing an amino acid sequence variation is predetermined, the mutation per se need not be predetermined. For example, in order to optimize the performance of a mutation at a given site, random mutagenesis may be conducted at the target codon or region and the expressed variants screened for the optimal combination of desired activity. Screening of the mutants is done using the assays described herein.

Substitutions, deletions, insertions or any combination thereof may be used to arrive at a final derivative. Amino acid substitutions are typically of single residues; insertions usually will be on the order of from about 1 to 20 amino acids, although considerably larger insertions may be tolerated. Deletions range from about 1 to about 20 residues, although in some cases deletions may be much larger. Generally, these changes are done on a few amino acids to minimize the alteration of the molecule. However, larger changes may be tolerated in certain circumstances. When small alterations in the characteristics of the STAT inhibitors are desired, substitutions are generally made by replacing a homologous amino acid for another in accordance with the following table:

| Original | Exemplary |
|---|---|
| Residue | Substitutions |
| Ala | Ser |
| Arg | Lys |
| Asn | Gln |
| Asp | Glu |
| Cys | Ser |
| Gln | Asn |
| Glu | Asp |
| Gly | Pro |
| His | Tyr |
| Ile | Leu, Val |
| Leu | Ile, Val |
| Lys | Arg |
| Met | Leu, Ile |
| Phe | Tyr, Trp |
| Ser | Thr |
| Thr | Ser |
| Trp | Tyr, Phe |
| Tyr | Trp, Phe |
| Val | Ile, Leu |

Homologous amino acids may be classified based on the size of the side chain and degree of polarization, including, small non-polar (e.g., cysteine, proline, alanine, threonine); small polar (e.g., serine, glycine, aspartate, asparagine); intermediate polarity (e.g., tyrosine, histidine, tryptophan); large non-polar (e.g., phenylalanine, methionine, leucine, isoleucine, valine). Homologous amino acid may also be grouped as follows: uncharged polar R groups (e.g., glycine, serine, threonine, cysteine, tyrosine, asparagine, glutamine); acidic amino acids (e.g., aspartic acid, glutamic acid); and basic amino acids (lysine, arginine, and histidine).

Accordingly, in one embodiment, the STAT inhibiting agents also include conservative variants of the amino acid sequence of FIG. 1 (SEQ ID NO:1). As indicated above, conservative variants refer to replacement of an amino acid residue by another homologous, biologically similar residue. Examples of convervative variations include, among others, substitution of a hydrophobic residue, such as isoleucine, valine, leucine, or methionine for another homologous amino acid; substitution of a polar residue for another homologous amino acid, such as arginine for lysine, glutamic acid for aspartic acid, or glutamine for arginine; substitution of an aromatic residue for another homologous amino acid, such as tyrosine for phenylalanine; etc.

Selecting substitutions that are less conservative than those shown in Table I makes substantial changes in function or immunological identity. For example, substitutions may be made that more significantly affect the structure of the polypeptide backbone in the area of the alteration of the alpha-helical or beta-sheet structure, the charge or hydrophobicity of the molecule at the target site, or the bulk of the side chain. In general, the substitutions expected to produce the greatest changes in the polypeptide's properties are those in which (a) a hydrophilic residue, e.g., seryl or threonyl, is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histidyl) is substituted for (or by) an electronegative residue (e.g., glutamyl or aspartyl); or (d) a residue having a bulky side chain (e.g., phenylalanine) is substituted for (or by) one not having a side chain (e.g., glycine).

In another embodiment, variants of STAT inhibiting agents as used herein include proteins encoded by nucleic acids containing codons replaced with degenerate codons coding for the same amino acid. This arises from the degeneracy of the genetic code where the same amino acids are encoded by alternative codons. Replacing one codon with another degenerate codon changes the nucleotide sequence without changing the amino acid residue. An extremely large number of nucleic acids may be made, all of which encode the inhibiting agents of the present invention. Thus, having identified a particular amino acid sequence, those skilled in the art could make any number of different nucleic acids by modifying the sequence of one or more codons in a way that does not change the amino acid sequence of the protein. In this regard, the present invention has specifically contemplated each and every possible variation of polynucleotides that could be made by selecting combinations based on the possible codon choices, and all such variations are to be considered specifically disclosed and equivalent to the sequence of FIG. 2. It also should be noted that codon optimization that results in one or small number of amino acid changes, particularly, conservative changes are also possible.

Changing the codons may be desirable for a variety of situations. For example, substitutions with a degenerate codon is useful when eliminating cryptic splice signals present in the coding regions of a gene, inserting restriction sites in the gene, distinguishing between one version of the same gene from another (e.g., by hybridization), creating alternative primers for amplification reactions, examining mutational bias in genes, changing chromosomal methylation patterns (e.g., for determining preferential parental transmission), and changing the expression levels of the gene of interest.

Accordingly, in a further embodiment, the inhibiting agent variants are codon optimized for expression in a particular organism. By “codon optimized” herein is meant changes in the codons of the gene of interest to those preferentially used in a particular organism such that the gene is efficiently expressed in the organism. Although the genetic code is degenerate in that most amino acids are represented by several codons, called synonyms or synonymous codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. Although codon bias may arise from nucleotide composition or mutational biases in different organisms, codon usage bias in bacteria and yeast correlates with the abundance of tRNA species in the cell. In general, codon bias is often associated with the level of gene expression. That is, certain codons are preferentially represented in the protein coding regions of highly expressed gene products. Thus, changing the codons to the preferred codons of a particular organism may allow higher level expression of the encoded protein in that organism. In this regard, the present embodiments of STAT inhibiting agent encompasses variants whose codons are altered to the preferred codons of the organism in which the gene of interest is being expressed. In other words, codons are preferably selected to fit the host cell in which the protein is being produced. For example, preferred codons used in bacteria are used to express the STAT inhibiting agents in bacteria; preferred codons used In yeast are used for expression in yeast; and preferred codons used in mammalian cells are used for expression in mammalian cells.

By “preferred”, “optimal” or “favored” codons, or “high codon usage bias” or grammatical equivalents as used herein is meant codons used at higher frequency in the protein coding regions than other codons that code for the same amino acid. The preferred codons may be determined in relation to codon usage In a single gene, a set of genes of common function or origin, highly expressed genes, the codon frequency in the aggregate protein coding regions of the whole organism, codon frequency in the aggregate protein coding regions of related organisms, or combinations thereof.

In a one embodiment, preferred or favored codons are determined for genes of common function, while in a more preferred embodiment, preferred codons are determined for protein coding regions of the whole organism or related organisms. In a most preferred embodiment, codon usage in a representative number of highly expressed gene products of an organism or related organisms will provide the basis for determining the set of preferred codons. Thus, in one aspect, preferred codons are those codons whose frequency increases with the level of gene expression. Since gene expression may be restricted to specific cells or certain developmental time periods (e.g., embryonic and adult), whether a gene is highly expressed is measured in respect to the cells and the temporal periods when the gene is expressed.

In another aspect, preferred codons are further delineated with respect to the size of the protein coding regions examined. Studies of codon bias show a negative correlation between the size of the protein and codon usage (see Duret, L. et al., Proc. Natl. Acad. Sci. USA 96: 44824487 (1999)). For proteins of increasing length, there is a tendency for less codon usage bias while highly expressed proteins of decreasing length display increased codon usage bias. Thus, in a preferred embodiment, the size of proteins used for assessing preferred codons includes proteins of all lengths, while a more preferred embodiment uses protein lengths up to about 550 amino acids. In the most preferred embodiment, proteins lengths of up to about 335 amino acids are used.

A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see, GCG CodonPreference, Genetics Computer Group Wisconsin Package; CodonW, John Peden, University of Nottingham; McInerney, J. O., Bioinformatics 14: 372-373 (1998); Stenico, M. et al., Nucleic Acids Res. 22:2437-2446 (1994); Wright, F., Gene 87: 23-29 (1990)). Codon usage tables are available for a growing list of organisms (see for example, Wada, K. et al., Nucleic Acids Res. 20: 2111-2118 (1992); Nakamura, Y. et al., Nucleic Acids Res. 28:292 (2000); Duret, et al., supra). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., Methods Enzymol. 266:259-281 (1996); and Tiwari, S. et al., Comput. Appl. Biosci. 13:263-270 (1997)).

Depending upon their intended use, particularly for administration to mammalian hosts, the subject peptides may be modified or attached to other compounds for the purposes of incorporation into carrier molecules, changing peptide bioavailability, extend or shorten half-life, control distribution to various tissues or the blood stream, diminish or enhance binding to blood components, and the like. The subject peptides may be bound to these other components by linkers which are cleavable or non-cleavable in the physiological environment such as blood, cerebrospinal fluid, digestive fluids, etc. The peptides may be joined at any point of the peptide where a functional group is present, such as hydroxyl, thiol, carboxyl, amino, or the like. Desirably, modification will be at either the N-terminus or the C-terminus.

Accordingly, in one aspect, the subject peptides may be modified by covalently attaching polymers, such as polyethylene glycol, polypropylene glycol, carboxymethyl cellulose, dextran, polyvinyl alcohol, polyvinylpyrrolidine, polyproline, poly(divinyl-ether-co-maleic anhydride), poly(styrene-c-maleic anhydride), etc., in the manner set forth in U.S. Pat. Nos. 4,640,835; 4,496,689; 4,301,144; 4,670,417; 4,791,192 or 4,179,337. Water-soluble polymers, such as polyethylene glycol and polyvinylpyrrolidine are known to cause a decrease in clearance of attached compounds from the blood stream as compared to unmodified compounds. The modifications can also increase solubility in aqueous media and reduce aggregation of attached proteins.

One or both, usually one terminus of the inhibitory protein, may be substituted with a lipophilic group, usually aliphatic or aralkyl, of from 8 to 36, usually 8 to 24 carbon atoms and fewer than two heteroatoms in the aliphatic chain, the heteroatoms usually being oxygen, nitrogen and sulfur. As further described below, the chain may be saturated or unsaturated, desirably having not more than 3 sites, usually not more than 2 sites of aliphatic unsaturation. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. (see, e.g., U.S. Pat. No. 6,225,444, hereby incorporated by reference). Preferred are unbranched, naturally occurring fatty acids between 14-22 carbon atoms in length. Other lipohilic molecules include glyceryl lipids and sterols, such as cholesterol. The lipophilic groups may be reacted with the appropriate functional group on the STAT inhibiting agent in accordance with conventional methods, frequently during the synthesis on a support, depending on the site of attachment of the oligopeptide to the support. Lipid attachment is useful where proteins and peptide may be introduced into the lumen of the liposome, along with other therapeutic agents, for administering the agents into a host. Increasing lipophilicity is also known to increase transport of compounds across the endothelial cells, and therefore may be useful in promoting uptake into tissues and cells.

The terminal amino group or carboxyl group of the oligopeptide may be modified by alkylation, amidation, or acylation to provide esters, amides or substituted amino groups, where the alkyl or acyl group may be of from about 1 to 30, usually 1 to 24, preferably either 1 to 3 or 8 to 24, particularly 12 to 18, carbon atoms. The peptide or derivatives thereof may also be modified by acetylation or methylation to alter the chemical properties, for example lipophilicity. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine, arginine, and histidine side chains (see Creighton, T. E., Proteins: Structure and Molecular Properties, W.H. Freeman & Co. San Francisco, Calif., 1983).

In another aspect, the peptide Is preferably conjugated to small molecules for the detection and isolation of inhibiting agents, and to target or transport them into specific cells, tissues, and organs. Small molecule conjugates include haptens, which are substances that do not initiate an immune response when introduced by themselves into an animal. Generally, haptens are small molecules of molecular weight less than about 2 kD, and more preferably less that about 1 kD. Haptens include small organic molecules, including without limitation, p-nitrophenol, digoxin, heroin, cocaine, morphine, mescaline, lysergic acid, tetrahydrocannabinol, cannabinol, steroids, pentamidine, biotin, etc.). Binding of the hapten, for example for purposes of detection or purification, is done with hapten specific antibodies or specific binding partners, such as avidin which binds biotin.

Small molecules that target the conjugate to specific cells or tissues may also be used. It is known that attaching a biotin-avidin complex to a protein increases uptake of such modified protein across the endothelial cells. Linkage of proteins to carbohydrate moieties, for example to a β-glycoside through a serine residue on the protein, preferably a β-O linked glycoside, enhances transport of the glycoside derivative into endothelial cells via the glucose transporter (Polt, R. et al., Proc. Natl. Acad. Sci. USA 91: 7144-7118 (1994)). Both of these types of modifications are encompassed within the scope of the present invention.

In another aspect, the STAT inhibiting proteins, and in some circumstances the encoding nucleic acids, are conjugated to a wide variety of other peptides or proteins for a variety of purposes. In one embodiment, the subject agents are attached to other peptides or proteins for targeting the agents to cells and tissues, or adding additional functionalities to the protein. For targeting, the protein or peptide used for conjugation will be selected based on the cell or tissue being targeted for therapy. Generally, these proteins include antibodies directed to a receptor, or other cell surface molecule, and ligands that interact with a receptor. For instance, for targeting to the central nervous system, suitable carrier proteins Include, among others, antibodies against the transferrin receptor (see U.S. Pat. No. 5,527,527, hereby incorporated by reference); cationized albumin; met-enkephalin (see U.S. Pat. Nos. 5,442,043, 4902,505, and 4,801,575; incorporated by reference); and antibodies to human insulin receptor (see Pardridge, W. M. et al., Pharm. Res. 12: 807-816 (1995); incorporated by reference). In another embodiment, the STAT inhibiting agents are conjugated to intercellular delivery peptides, including protein transduction peptides, as further described below. The proteins may also compromise poly-amino acids including, but not limited to, polyarginine, polylysine, polyaspartic acid, etc., which may be incorporated into other polymers, such as polyethylene glycol, for preparation of vesicles or particles containing the conjugated proteins or nucleic acids.

In another aspect, the subject peptides may be expressed in conjunction with other peptides or proteins, so as to be a portion of the polypeptide chain, either internal, or at the N- or C-terminus to form chimeric proteins or fusion proteins. By “fusion polypeptide” or “fusion protein” or “chimeric protein” or “recombinant protein” herein is meant a protein composed of a plurality of protein components that are in their natural form not nomally joined together but are joined by the respective amino and carboxy termini through a peptide linkage to form a continuous polypeptide. Plurality in this context means at least two, and preferred embodiments generally utilize three to twelve components, although more may be used. It will be appreciated that the protein components can be joined directly or joined through a peptide linker/spacer as outlined below.

Fusion polypeptides may be made to a variety of peptides or proteins for targeting to cells and tissues, trafficking to intracellular compartments, tracking the fusion protein in a cell or an organism, and displaying the subject proteins in a conformationally restricted form. Proteins useful for generating fusion proteins include various reporter proteins, structural proteins, cell surface receptors, receptor ligands, toxins, and enzymes. Exemplary reporter proteins include fluorescent proteins (e.g., Aequoria victoria GFP, Renilla reniformis GFP, Renilla muelleri GFP, luciferases, etc., and variants thereof), β-galactosidase, alkaline phosphatase, E. coli maltose binding protein, coat proteins of filamentous bacteriophage (e.g., minor coat protein, pill, or the major coat protein, pVIII, etc.), T cell receptor, charybdotoxin, and the like.

Fusion proteins also encompass fusions with fragments of proteins or other peptides, either alone or as part of a larger protein sequence. Thus, the fusion polypeptides may comprise fusion partners. As used herein “fusion partners” are sequences that are associated with the peptide that confers all members of the proteins in that class a common function or ability. Fusion partners can be heterologous (e.g., not native to the host cell) or synthetic (e.g., not native to any cell). The fusion partners include, but are not limited to, a) targeting sequences, which direct the subject proteins to a tissue or cell, promote internalization by a cell, or mediate localization to a subcellular or extracellular compartment; b) presentation structures, which provide the subject proteins in a conformationally restricted or stable form; c) linker sequences, which conformationally decouples the oligopeptide from the fusion partner; and e) any combination of the above.

Suitable targeting sequences include, but are not limited to, binding sequences capable of causing binding of the expression product to a predetermined molecule or class of molecules while retaining bioactivity of the expression product; signal sequences capable of constitutively localizing the peptides to a predetermined cellular locale, including a) subcellular locations such as the Golgi, endoplasmic reticulum, nucleus, nucleoli, nuclear membrane, mitochondria, chloroplast, secretory vesicles, lysosome, periplasmic space, cellular membrane; and b) extracellular locations via a secretory signal. Particularly preferred is localization to either subcellular locations or to the outside of the cell via secretion.

In one embodiment, the targeting sequence is a secretory signal sequence capable of effecting the secretion of the STAT inhibiting protein. There are a large number of known secretory signal sequences which direct secretion of the peptide into the extracellular space when placed at the amino end relative to the protein of Interest. Secretory signal sequences and their transferability to unrelated proteins are well known (see, e.g., Silhavy, T. J. et al., Microbiol. Rev. 49: 398-418 (1985)). Secretion of the peptide is particularly useful for generating peptides capable of binding to the surface of, or affecting the physiology of target cells other than the host cell, e.g., the cell expressing the STAT inhibiting agent. In this manner, target cells grown in the vicinity of cells expressing the library of peptides are exposed to the secreted STAT inhibiting agent.

Accordingly, targeting of STAT Inhibiting proteins to the extracellular environment is also useful for intercellular delivery of the subject inhibitors to other cells. A combination of secretory signal sequence and/or a intercellular delivery or cellular uptake sequence allows synthesis and export of the peptide and uptake of the protein by another cell. In this regard, the subject STAT inhibiting agents may be fused to a variety of uptake or intercellular delivery peptides, including protein transduction peptides. As used herein, “intercellular delivery peptides” or “protein transduction peptides” are peptides which mediate translocation of the peptide into a cell, generally via membrane penetration. Protein transduction domains have no common structure except for the presence of basic amino acid residues (e.g., arginine and lysine), which appear to function In interacting with the cell membrane. In one embodiment, the protein transduction peptide comprises herpes simplex virus VP22 protein (Elliott, G. et al., Cell 88:223-233 (1997); Lai, Z. et al., Proc. Natl. Acad. Sci. USA 97:11297-11302 (2000)). VP22 is a basic, 38 kilodalton protein encoded by the viral UL49 gene. Fusion proteins containing VP22 is exported from the cytoplasm and taken up by neighbouring cells. This property provides a convenient means of delivering therapeutic peptides to cells. For the present purposes, STAT inhibiting proteins are expressed as fusion proteins with VP22 protein, or with functional fragments of VP22 protein. Amino acid sequences displaying intercellular delivery properties include amino acid residues at about 81-195 of VP22 (Aints, A. et al., Gene Ther. 8:1051-1056 (2001); hereby incorporated by reference). Various expression vectors encoding the fusion proteins may be constructed and delivered into cells, where expression will lead to delivery of the STAT inhibiting protein to neighbouring cells. In particular, delivery to tumor cells in vivo may allow delivery of the fusion protein to other neighbouring tumor cells, especially where the tumor is a solid tumor. Alternatively, fusion proteins are recombinantly or chemically synthesized and then introduced into cells to inhibit STAT activity.

In another embodiment, the intercellular delivery sequence comprises a human immunodeficiency virus (HIV) Tat protein, or Tat related protein (Fawell, S. et al., Proc. Natl. Aced. Sci. USA 91:664-668 (1994); Nagahara, H. et al., Nat. Med. 4:1449-1452 (1998); references incorporated by reference). The HIV Tat protein is 86 amino acids long, containing a highly basic region and a cysteine rich domain. Although Tat is primarily known as a transcriptional activator of the viral long terminal repeat (LTR), it also displays membrane penetrating activity (Fawell, S. et al., supra). Tat peptides of about 11 amino acids (e.g., amino acid residues 48-60) are sufficient for protein transduction activity. Thus, in one aspect, fusions between Tat proteins and STAT inhibiting peptides are made. In a further embodiment, the intercellular delivery peptide is a branched structure containing multiples copies of Tat sequence RKKRRQRRR (Tung, C. H. et al., Bioorg. Med Chem 10:3609-3614 (2002))

Other peptides useful as protein transduction peptides include, without limitation, the third alpha helix of Drosophila Antennapedia homeodomain protein, transportan, amphipathic peptide carriers (Morris, M. C. et al., Nat. Biotechnol. 19:1173-1176 (2001); polyarginine heptapeptide R7 (Chen, L. et al., Chem. Biol. 8:1123-1129 (2001)) and other arginine rich peptides (Futaki, S. et al., J. Biol. Chem. 276:5836-5840 (2001).

The STAT inhibitory proteins may be targeted to various intracellular locations by the use of appropriate targeting fusion partners. In one aspect, targeting to nucleus is achieved by use of nuclear localization signals (NLS). NLSs are generally short, positively charged domains that direct the proteins having the NLSs to the cell nucleus. Typical NLSs sequences include the single basic NLSs of SV40 large T antigen (Kalderon, D. et al., Cell 39: 499-509 (1984)); human retinoic acid receptor-β nuclear localization signal (NF-KB p50 and p65 (Ghosh, S. et al., Cell 62: 1019-1029 (1990) and Nolan, G. et al., Cell 64: 961-999 (1991)); and the double basic NLSs' as exemplified by nucleoplasmin (Dingwall, C. et al., J. Cell Biol. 107: 641-649 (1988)).

In another aspect the targeting sequences are membrane anchoring sequences. Proteins are directed to the membrane via signal sequences and stably incorporated in the membrane through a hydrophobic transmembrane domain (designated as TM). The TM segments is positioned appropriately on the expressed fusion protein to display the subject peptide either intracellularly or extracellularly, as is known in the art. Membrane anchoring sequences and signal sequences include, but are not limited to, those derived from (a) class I integral membrane proteins such as IL-2 receptor β-chain (Hatakeyama, M. et al., Science 244: 551-556 (1989)) and insulin receptor β-chain (Hetekayama, M. et al, supra), (b) class II integral membrane proteins such as neutral endopeptidase (Malfroy, B. et al Biochem. Biophys. Res. Commun. 144: 59-66 (1987)), and (c) type III proteins such as human cytochrome P450 NF25 (Hetekayama, M. et al, supra); and those from CD8, ICAM-2, IL-8R, and LFA-1.

Membrane anchoring sequences also include the GPI anchor, which produces a covalent bond between the GPI anchor sequence and the lipid bilayer via a glycosyl-phosphatidylinositol. GPI anchor sequences are found in various proteins, including Thy-1 and DAF (Homans, S. W. et al., Nature 333: 269-272 (1988)). Similarly, acylation sequences allow for attachment of lipid moieties, e.g., isoprenylation (e.g., farnesyl and geranyl-geranyl; see Farnsworth, C. C. et al., Proc. Natl. Acad. Sci. USA 91:11963-11967 (1994) and Aronheim, A. et al., Cell 78: 949-961 (1994)), myristoylation (Stickney, J. T., Methods Enzymol. 332:64-77 (2001)), or palmitoylation. In one aspect, the subject protein will be bound to a lipid group at a terminus, so as to be able to be bound to a lipid membrane, such as a liposome.

Other intracellular targeting sequences are lysozomal targeting sequences (e.g., sequences In LAMP-1 and LAMP-2; Uthayakumar, S. et al., Cell Mol. Biol. Res. 41:405-420 (1995) and Konecki, D. S. et al., Biochem. Biophys. Res. Comm. 205:1-5 (1994)); mitochondrial localization sequences (e.g., mitochondrial matrix sequences, mitochondrial inner membrane sequences, mitochondrial intermembrance sequences, or mitochondrial outer membrane sequences; see Shatz, G., Eur. J. Biochem. 165:1-6 (1987)); endoplasmic recticulum localization sequences (e.g., calreticulin, Pelham, H. R., Royal Soc. London Transactions B:1-10 (1992); adenovirus E3/19K protein, Jackson, M. R. et al., EMBO J. 9:3153-3162 (1990)); and peroxisome localization sequences (e.g., luciferase peroxisome matrix sequence, Keller, G. A. et al., Proc. Natl. Acad. Sci. USA 4:3264-3268 (1987)).

For displaying the protein or peptide to enhance binding interactions, particularly when fragments of STAT inhibiting agents are used, the fusion partner is a presentation structure. By “presentation structure” as used herein is meant a sequence that when fused to the subject peptides presents the peptides in a conformationally restricted form. Preferred presentation structures display a peptide on an exterior surface, such as a peptide loop, to promote binding interactions with the exposed peptide surface. Generally, such presentation structures comprise a first portion joined to the N-terminus of a protein and a second portion joined to the C-terminal end of the protein. That is, the protein of interest is inserted into the presentation structures. Preferably, the presentation structures are selected or designed to have minimal biological activity when expressed in the target cells.

Preferably, the presentation structures maximize accessibility to the peptides by displaying or presenting the peptide or an exterior loop. Suitable presentation structures include, but are not limited to, coiled coil stem structures, minibody structures, loops on β-turns, dimerization sequences, cytokine linked structures, transglutaminase linked structures, cyclic peptides, helical barrels, leucine zipper motifs, etc.

In one embodiment, the presentation structure is a coiled-coil structure, which allows presentation of the subject peptide on an exterior loop (Myszka et al., Biochemistry 33:2362-2373 (1994)), such as a coiled-coil leucine zipper domain (Martin et al., EMBO J. 13:5303-5309 (1994)). The presentation structure may also comprise minibody structures, which is essentially comprised of a minimal antibody complementarity region. The minibody structure generally provides two peptide regions that are presented along a single face of the tertiary structure in the folded protein (Bianchi et al., J. Mol. Biol. 236:649-659 (1994); Tramontano, A. et al., J. Mol. Recognit. 7:9-24 (1994)).

In another aspect, the presentation structure comprises two dimerization sequences. The dimerization sequences, which can be same or different, associate non-covalently with sufficient affinity under physiological conditions to structurally constrain the displayed peptide; that is, if a dimerization sequence is used at each terminus of the subject protein, the resulting structure can display the protein In a structurally limited form. A variety of sequences are suitable as dimerization sequences (see, e.g., WO 99/51625). Any number of protein-protein interaction sequences known in the art are useful.

In a further aspect, the presentation sequence confers the ability to bind metal ions to generate a conformationally restricted secondary structure. Thus, for example, C₂H₂ zinc finger sequences are used. C₂H₂ sequences have two cysteines and two histidines positioned such that a zinc ion is chelated. Zinc finger domains are known to occur independently in multiple zinc-finger peptides to form structurally independent, flexibly linked domains (Nakaseko, Y. et al., J. Mol. Biol. 228:619-636 (1992)). A general consensus sequence is (5 amino acids)-C-(2 to 3 amino acids)-C-(4 to 12 amino acids)-H-(3 amino acids)-H-(5 amino acids). A preferred example would be —FQCEEC-random peptide of 3 to 20 amino acids-HIRSHTG. Similarly, CCHC boxes having a consensus sequence —C-(2 amino acids)-C-(4 to 20 random peptide)-H-(4 amino acids)-C— can be used, (see Bavoso, A. et al., Biochem. Biophys. Res. Commun. 242:385-89 (1998)). Other examples include (1) -VKCFNC-4 to 20 random amino acids-HTARNCR—, based on the nucleocapsid protein P2; (2) a sequence modified from that of the naturally occurring zinc-binding peptide of the Lasp-1 LIM domain (Hammarstrom, A. et al., Biochemistry 35:12723-12732 (1996)); and (3)-MNPNCARCG-4 to 20 random amino acids-HKACF—, based on the NMR structural ensemble 1ZFP (Hammarstrom, A. et al., supra).

In yet another aspect, the presentation structure is a sequence that comprises two or more cysteine residues, such that a disulfide bond may be formed, resulting in a conformationally constrained structure. That is, use of cysteine containing peptide sequences at each terminus of the subject proteins results in cyclic structures, as described above. A cyclic structure reduces susceptibility of the presented peptide to proteolysis and increases accessibility to its target molecules. As will be appreciated by those skilled in the art, this particular embodiment is particularly suited when secretory targeting sequences are used to direct the peptide to the extracellular space.

In another embodiment, the fusion partner comprises a tag polypeptide, which provides an epitope to which an anti-tag antibody can selectively bind. The epitope tag is generally placed at the amino- or carboxyl-terminus of the STAT inhibitory protein. The presence of such epitope-tagged forms can be detected using an antibody against the tag polypeptide. Also, provision of the epitope tag enables the inhibitory protein to be readily purified by affinity purification using an anti-tag antibody or another type of affinity matrix that binds to the epitope tag. In an alternative embodiment, the chimeric molecule may comprise a fusion of a STAT inhibiting agent with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Mol. Cell. Biol. 5:3610-3616 (1985)); and the Herpes Simplex virus glycoproteln D (gD) tag and its antibody (Paborsky et al., Protein Engineering, 3:547-553 (1990)). Other tag polypeptides include the Flag-peptide (Hopp et al., BioTechnology, 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

The fusion partners may be placed anywhere (e.g., N-terminal, C-terminal, internal loops) in the structure as the biology and activity permits. In addition, more than one fusion partners may be joined to the STAT inhibiting protein. Thus, for example, the STAT Inhibiting protein may contain a targeting sequence (either N-terminal region, C-terminal region, or internal region, as described above) at one location, and a protein transduction domain in the same place or a different place on the molecule. Thus, any combination of fusion partners may be made to provide multiple functions and properties to the fusion protein.

The fusion polypeptide may also include a linker or spacer sequence. Linker sequences between various targeting sequences, for example, membrane targeting sequences, and the other components of the constructs, such as the STAT inhibiting agent, may be desirable to allow unhindered interaction of the peptides with potential targets. Useful linkers include glycine polymers (G)ₙ, glycine-serine polymers (including, for example, (GS)ₙ, (GSGGS)ₙ and (GGGS)ₙ, where n is an integer of at least one), glycine-alanine polymers, alanine-serine polymers, and other flexible linkers, such as the tether for the shaker potassium channel, as will be appreciated by those in the art. Glycine and glycine-serine polymers are preferred since both of these amino acids are relatively unstructured, and therefore may be able to serve as a neutral tether between components.

In one embodiment, the peptide is connected to the STAT inhibiting peptides via linkers. That is, while one embodiment utilizes the direct linkage of the fusion partners, another embodiment may utilize linkers at one or both ends of the inhibiting peptide. When attached either to the N- or C-terminus, one linker may be used. When the STAT inhibiting protein is inserted in an internal position, as is generally outlined above, preferred embodiments utilize at least one linker and preferably two, one at each terminus of the protein. Linkers are generally preferred for conformationally decoupling any fusion partner from the STAT inhibiting protein to minimize local structural distortions and attenuation of inhibiting activity.

For expressing the STAT inhibiting agents, the present invention further relates to fusion nucleic acids or recombinant nucleic acids that encode and express the proteins described above. By “fusion nucleic acid” herein is meant a plurality of nucleic acid components that are joined together, either directly or indirectly. As will be appreciated by those in the art, in some embodiments the sequences described herein may be DNA, for example when extrachromosomal plasmids are used, or RNA when retroviral vectors are used. In some embodiments, the sequences are directly linked together without any linking sequences while in other embodiments linkers such as restriction endonuclease cloning sites, linkers encoding flexible amino acids, such as glycine or serine linkers such as known in the art, are used, as discussed above.

In the present invention, the fusion nucleic acids comprise expression vectors for expressing the proteins of the present invention. The expression vectors may be either self-replicating extrachromosomal vectors or vectors which integrate into a host genome. Generally, these expression vectors include control sequences operably linked to the nucleic acid encoding the protein. The term “control sequences” refers to DNA sequences necessary for the expression of an operably linked coding sequence in a particular host organism. Thus, control sequences include sequences required for transcription and translation of the nucleic acids, which are selected in reference to the target organism used for expressing the proteins. For example, for prokaryotes, the sequences include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. In the present context, operably linked means that the control sequences, such as transcription and translation regulatory sequences, are positioned relative to the coding sequence in such a manner that expression of the encoded protein occurs. For example, a promoter or enhancer is operably linked to a coding sequence if it affects the transcription of the sequence; or a ribosome binding site is operably linked to a coding sequence if it is positioned so as to facilitate translation. Where the fusion nucleic acid encodes a fusion protein, for example a protein linked to a secretory leader sequence, the DNA for the secretory leader is operably linked to DNA for a polypeptide if it is expressed in a manner resulting in secretion of the polypeptide.

In general, the transcriptional and translational regulatory sequences may include, but are not limited to, promoter sequences, enhancer or transcriptional activator sequences, ribosomal binding sites, CAP sequences, transcriptional start and stop sequences, and translational start and stop sequences. In a preferred embodiment, the regulatory sequences include a promoter and transcriptional start and stop sequences.

Promoter sequences are either constitutive or inducible promoters. By “promoter” herein is meant nucleic acid sequences capable of Initiating transcription of the fusion nucleic acid or portions thereof. Promoters may be constitutive wherein the transcription level is constant and unaffected by modulators of promoter activity. Promoter may be inducible in that promoter activity is capable of being increased or decreased, for example as measured by the presence or quantitation of transcripts or translation products (Walter, W. et al., J. Mol. Med. 74: 379-392 (1996)). Promoters may also be cell specific wherein the promoter is active only in particular cell types. Thus, promoter as defined herein includes sequences required for initiating and regulating the transcription level in cells and transcription in specific cell types. Furthermore, the promoters may be either naturally occurring promoters, hybrid promoters that combine elements of more than one promoter, or synthetic promofers based on consensus sequence of known promoters.

The fusion nucleic acid comprising the expression vector may comprise additional elements. For example, the expression vector may have two replication systems, thus allowing it to be propagated in two organisms, for example in mammalian or insect cells for expression and in a prokaryotic host for cloning and amplification. Furthermore, for integrating into the host chromosomal elements, the expression vector may contain sequences necessary for the integration process. Generally, the integration sequences used will depend on the integration mechanism. For homologous recombination, a sequence homologous to specific regions of a host cell genome is incorporated into the fusion nucleic, as is well known in the art. Preferably, two homologous sequences flank the expression construct or the region to be inserted into the genome. By selecting the appropriate homologous sequence, the vector may be directed to specific regions of the host cell genome. Alternatively, integration is directed by inclusion of sequences necessary for site-specific recombination. A variety of site-specific recombination systems are known. The cre-lox system comprises the Cre recombinase of bacteriophage P1 which catalyzes recombination between short 34 basepair lox-P sites. Presence of lox-P sites on two different DNAs results in recombination between the two lox-P sites, thus generating a single recombinant containing two lox-P sites flanking the integrated DNA (see for example, Fukushige, S. et al., Proc. Natl. Aced. Sci. USA 89:7905-7909 (1992)). Cre-lox recombinations can function in any cell system containing lox-P sites and Cre recombinase. Insertion of lox-P sites into the genome of organisms and expression of Cre allows for recombination events in bacterial, yeast, plant, and mammalian cells (Sauer, B., Nucleic Acids Res. 24:4608-4613 (1996); Araki, K. et al., Nucleic Acids Res. 25:868-872 (1997); and Vergunst, A. C., Plant Mol. Biol. 38:393-406 (1998); U.S. Pat. No. 4,959,317).

Other systems applicable for integrating the expression vectors include, but are not limited to, the flp recombinase system (see, e.g., U.S. Pat. No. 6,140,129), the λ integrase system, bacteriophage phage Mu, transposon systems (e.g., γδ), retroviral vectors, and the like. As some of the integration mechanisms function only in certain organisms, the appropriate integration system is selected according to the cells in which the expression vectors are used. In another preferred embodiment, the site-specific recombination sites are not used for integration but for deletion or rearrangement of nucleic acid sequences on the fusion nucleic acid. Deletion or rearrangement is accomplished by appropriate position of the recombination sequences, which are well known in the art.

In one embodiment, the expression vector also contains a selectable marker gene to allow the selection of transformed host cells. Generally, the selection will confer a detectable phenotype that provides a way of differentiating between cells that express and do not express the selection gene. Selection genes are well known in the art and will vary with the host cell used, as further described below.

Accordingly, using the nucleic acids of the present invention, a variety of expression vectors are made for encoding and expressing the proteins described above. As used herein, the term “vector” includes plasmids, cosmids, artificial chromosomes, viruses, and the like. In one embodiment, the expression vectors are bacterial expression vectors including vectors for Bacillus subtilis, E. coli, Haemophilus, Streptococcus cremoris, and Streptococcus lividans, among others. These vectors are well known in the art. A suitable bacterial promoter is any nucleic acid sequence capable of binding bacterial RNA polymerase and initiating the downstream (3′) transcription of the coding sequence of the fusion protein into mRNA. A bacterial promoter has a transcription initiation region, which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage (e.g., pL) may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the fusion protein in bacteria. The signal sequence typically encodes a signal peptide comprised of hydrophobic amino acids which direct the secretion of the protein from the cell. The protein is either secreted into the growth media (e.g., in gram-positive bacteria) or into the periplasmic space, located between the inner and outer membrane of the cell (e.g., in gram-negative bacteria).

The bacterial expression vector may also include a selectable marker gene to provide a basis for selecting the bacterial strains that have been transformed. Suitable selection genes include, without limitation, genes, which render the bacteria resistant to drugs, such as ampicillin, chloramphenicol, erythromycin, kanamycin, neomycin and tetracycline; and reporter genes such as green fluorescent protein, luciferase, or β-galactosidase. Selectable markers also include biosynthetic genes, such as those in the histidine, tryptophan and leucine biosynthetic pathways. These components are assembled into expression vectors and introduced in bacterial host cells, using techniques well known in the art (e.g., calcium chloride treatment, electroporation, etc.).

In another embodiment, the expression vectors are used to express the proteins in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL promoters (e.g., GAL 1, GAL 4, GAL 10. etc.), the promoters from alcohol dehydrogenase (ADH or ADC1), enolase, glucokinase, glucose-6-phosphate isomerase, glyceraldehyde-3-phosphate-dehydrogenase, hexokinase, phosphofructokinase, 3-phosphoglycerate mutase, pyruvate kinase, fructose bisphosphate, acid phosphatase gene, tryptophase synthase (TRP5) and copper inducible CUP1 promoter. Any plasmid containing a yeast compatible promoter, an origin of replication, and termination sequences is suitable.

Yeast selectable markers include, without limitation, genes complementing mutations ADE2, HIS4, LEU2, TRP1, URA3, and genes conferring resistance to tunicamycin (ALG7 gene), G418 (neomycin phosphotransferase gene), growth in presence of copper ions (metallothionein CUP1 gene), resistance to fluoroacetate, (fluoroacetate dehalogenase), or resistance to formaldehyde (formaldehyde dehydrogenase).

In another embodiment, the expression vectors are used for expression in plants, particularly for large scale production of recombinant STAT inhibiting agents. Plant expression vectors are well known in the art. Vectors are known for expressing genes in Arabidopsis thaliana, tobacco, carrot, and maize and rice cells. Suitable promoters for use in plants include those of plant or viral origin, including, but not limited to CaMV 35S promoter (active in both monocots and dicots, Chapman, S. et al., Plant J. 2, 549-557 (1992)) nopoline promoter, mannopine synthase promoter, soybean or Arabidopsis thaliana heat shock promoters, tobacco mosaic virus promoter (Takmatsu, et al., EMBO J. 6: 307 (1987)), AT2S promoters of Arabidopsis thaliana (e.g., PAT2S1, PATS2, PATS3 etc.). In a further embodiment, the promoters are tissue specific promoters active in specific plant tissues or cell types (e.g., roots, leaves, shoot meristem etc.), which are well known in the art. Alternatively, the expression vectors comprise recombinant plasmid expression vectors based on Ti plasmids or root inducing plasmids.

In another aspect, regulatory sequences include “enhancers” to regulate expression. Preferably these are of plant, bacterial (e.g. Agrobacterium), viral origin specific to plants. The enhancers may act at either at the transcriptional or translational level. The fusion nucleic acids may also comprise one or more introns, preferably of plant origin, to increase the efficiency of expression of the fusion nucleic acid. For example, insertion of an intron into the 5′ untranslated sequence of a gene (e.g., between site of transcription initiation and translation initiation) leads to increased stability of the messenger RNA. The intron is preferably, though not necessarily, the first intron.

Optionally, a selectable marker gene is used with the expression vectors. The marker may be a drug resistance gene, a herbicide resistance gene, or any other selectable marker that can be used for selecting plant cells transformed with the vector. Suitable plant markers include adenosine deaminase, dihydrofolate reductase, hygromycin transferase, bar gene (Lohar, D. P., J. Exp. Bot. 52:1697-702 (2001)), green fluorescent proteins (including rGFP and pGFPs of the present invention), amino-glycoside 3′-O-phosphotransferase II (e.g., kanamycin, neomycin, and G418 resistance).

In addition, the plant expression vectors may comprise plant specific targeting sequences in addition to the targeting sequences described above. In one aspect, the sequences are chloroplast or mitochondrial targeting sequences. An example of a chloroplast targeting signals is the small subunit of ribulose 1,5 diphosphate of Pisum sativum. For a mitochondrial targeting sequence, an example is the precursor of the beta subunit of mitochondrial ATPase F1 of Nicotiana plumbaginifolia. In another aspect, the targeting signal comprises a vacuolar targeting sequences or “propeptide”. These sequences target the proteins to vacuoles of aqueous tissues, including leaves or protein bodies of storage tissues (Neuhaus, J. M et al. (1991) Proc. Natl. Acad. Sci. USA 88: 10362-66; and Sebastiani, F. L. et al. (1991) Eur. J. Biochem. 199: 441-50).

In another embodiment, the expression vectors are used to express the proteins insect cells. Expression vectors for the transformation of insect cells, and in particular, baculovirus vectors used to create recombinant baculoviruses for expressing foreign genes, are well known in the art (see for example, O'Reilly, D. R. et al. “Baculovirus Expression Vectors: A Laboratory Manual,” W.H. Freeman & Co, New York, 1992). By “baculovirus” or “nuclear polyhedrosis viruses” as used herein is meant expression systems using viruses classified under the family of baculoviridae, preferably subgroup A. These include expression systems specific for Bombix, Autographica, and Spodoptera (see for example, U.S. Pat. No. 5,194,376). Other expression systems include Amsacta moorei entomopoxvirus (AmEPV), Aedes aegypti desonucleosis (Aedes DNV, U.S. Pat. No. 5,849,523), and Galleria mellonella densovirus (GmDNV, Tal, et al., Arch. Insect Biochem. Physiol. 22: 345-356 (1993)). In another embodiment, expression vectors comprise fusion nucleic acids that integrate Into the host chromosome. This may be achieved by homologous recombination, particularly modified homologous recombination techniques when the insect cells or insect do no t readily undergo homologous recombination (see Rong, Y. S., Science 288: 2013-18 (2000)); site directed recombination (e.g., cre-lox); and transposon mediated integration (e.g., P-element transposition elements, etc.).

Promoters suitable for controlling expression in insects include Autographa californica nuclear polyhdrosis virus polyhedrin promoter, heat shock promoter (e.g., hsp 70), tubulin promoter, p10 promoter, Aedes DNV viral p7 and p61 promoters. In one embodiment, the promoter allows expression at an early stage in viral infection and/or allows expression in substantially all tissues of an Insect. In another embodiment, the promoter is a cell specific and developmental stage specific promoter, many of which are well known in the art. An example of a developmental stage specific promoter is the ecdysone regulated promoters that are active during molting and larval/pupal stages because of increases in the steroid hormone ecdysone during these developmental periods. Cell specific promoters include promoters active in the nervous system (e.g., ELAV), imaginal discs, gut, malphigian tubules, antennae (e.g, odor binding protein gene promoter), etc.

Although mammalian targeting sequences function in insect cells, targeting sequences derived from insect genes are preferred under some circumstances, for example to efficiently express secreted or membrane bound proteins in Insect cells. Signal sequence include Manduca sexta AKH signal peptide sequence, Drosophila cuticle protein signal peptides (e.g., CP1, CP2, CP3 and CP4, U.S. Pat. No. 5,278,050), and honey bee mellitin excretion peptide (MKFLVDVALVFMWYISYIYA) (SEQ ID NO: 74).

In a further embodiment, the expression vectors are used for expression In animals, such as fish, birds, and especially mammals, such as rodents, pigs, and primates. A variety of expression vectors are known for expressing proteins in animal cells, Including fusion nucleic acids existing extrachromosomally, as integrants in the host chromosome, or as viral nucleic acids. Viral expression vectors may be based on vectors derived from adenovirus, lentivirus, alphavirus, poxvirus (vaccinia virus), papilloma virus (e.g., bovine papilloma virus), or retrovirus, as further described below.

The expression vectors may include inducible and constitutive promoters for expressing the nucleic acids encoding the STAT inhibiting agents. A mammalian promoter will have a transcription initiating region, generally located 5′ to the start of the coding region, and a TATA box, present at about 25-30 basepairs upstream of the transcription initiation site. The promoter will also contain upstream regulatory elements that control the rate and initiation of transcription, including CAAT and GC box, enhancer sequences, and repressor/silencer sequences (see for example, Chang, B. D., Gene 183: 137-142 (1996)). These promoter controlling elements may act directionally, requiring placement upstream of the promoter region, or act non-directionally. These aforementioned transcriptional control sequences may be provided from non-viral or viral sources. Commonly used promoters and enhancers are from viral sources since the viral genes have a broad host range and produce high expression rates. Viral promoters, including upstream controlling sequences, may be from polyoma virus, adenovirus 2, simian virus 40 (early and late promoters), herpes simplex virus (e.g., HSV thymidine kinase promoter), human cytomegalovirus promoter (CMV), mouse mammary tumor virus (MMTV-LTR) promoter; and the like. A variety of non-viral promoters with constitutive, inducible, cell specific, or developmental stage specific activities are also well known in the art (e.g., β-globin promoter, mammalian heat shock promoter, metallothionein, ubiquitin C promoters, EF-1 alpha promoters, etc.). Cell specific promoters, which are well known in the art, include promoters active in specific cells including, but not limited to brain, olfactory bulb, thyroid, lung, muscle, pancreas, liver, lung, heart, breast, prostate, kidney, etc. Promoters and promoter controlling elements are chosen based on the desired level of promoter activity and the cell type in which the proteins of the present invention are to be expressed.

Generally, these vectors also include selectable marker genes. Suitable marker genes include reporter or selection genes as further described below. Selection genes include, but are not limited to neomycin, blastocidin, bleomycin, puromycin, hygromycin, and multiple drug resistance (MDR) genes. Suitable reporter genes include, but are not limited to, fluorescent proteins (e.g., green fluorescent proteins, luciferases) enzymatic markers (e.g., P-galactosidase, glucouronidase, alkaline phosphatase etc.), and surface proteins (e.g., CD8).

Additional sequences in the expression vectors include splice sites for proper expression, polyadenylation signals, 5′ CAP sequence, transcription termination sequences, and the like. Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation. Examples of transcription termination and polyadenylation signals include those derived from SV40.

Other sequences may include centromere sequences for generating artificial chromosomes for delivering larger fragments of DNA than can be contained and expressed in a plasmid or viral vector. Artificial chromosomes of 6 to 10M bp are constructed and delivered via conventional delivery methods (e.g., liposomes, polycationic amino polymers, or other carriers) for therapeutic purposes. The choice and design of an appropriate vector is within the ability and discretion of one of ordinary skill in the art.

In a preferred embodiment, the expression vectors are viral based expression systems. These include as non-limiting examples retroviral, lentiviral, adenoviral, adenoviral-associated viral (AAV), alphaviral, and papilloma viral vectors. In one embodiment, the expression vectors are retroviral vectors. By “retroviral vectors” herein is meant vectors used to introduce into appropriate hosts the nucleic acids of the present invention in the form of a RNA viral particle generated by retroviral genomes. Retroviruses are single stranded RNA viruses which convert its RNA to double stranded DNA by action of reverse transcriptase. The DNA integrates into the host chromosome to generate a proviral integrant, which then directs synthesis of viral RNA. A vector containing retroviral long terminal repeats (LTR) and T packaging sequence, when introduced into an appropriate packaging cell line, synthesizes RNA transcripts which are then inserted into viral particles and extruded into the culture medium. By replacing parts of the viral genome (e.g., gag, pol, and env) with a nucleic acid of interest or a heterologous gene, such as the nucleic acid sequence for a STAT inhibiting agent, expression of the nucleic acid of interest is achieved. As used herein, a heterologous region is a nucleic acid segment within a larger molecule that is not found in association with the larger molecule in nature. For example, a heterologous gene encoding a mammalian protein is flanked by nucleic acid sequences that do not flank the mammalian gene encoding the protein in the genome source of the organism. A synthetic sequence not found in nature will also constitute a heterologous sequence.

A variety of retroviral vectors are known in the art. Preferred retroviral vectors include a vector based on the murine stem cell virus (MSCV) (Hawley, R. G. et al., Gene Ther. 1:136-138 (1994)) and a modified MFG virus (Riviere, I. et al., Proc. Natl. Acad. Sci. USA 92: 6733-6737 (1995)), and pBABE (Morgenstern J. P., Nucleic Acids Res. 18:3587-3596 (1990). In addition, particularly well suited retroviral transfection systems for generating retroviral vectors are described in Mann, et al., Cell 33:153-159 (1983); Pear, W. S. et al., Proc. Natl. Aced. Sci. USA 90:8392-8396 (1993); Kitamura, T. et al., Proc. Natl. Acad. Sc. USA 92:9146-50 (1995); Kinsella, T. M. et al., Hum. Gene Ther. 7: 1405-1413 (1996); Hofmann, A. et al., Proc. Natl. Acad. Sci. USA 93: 5185-5190 (1996); and Choate, K. A. et al., Hum. Gene Ther. 7:2247-53 (1996); all of which are incorporated by reference. Generally, the retroviral vectors are used to express the nucleic acids of the present invention in proliferating cells.

In addition, the retroviral vectors may incorporate the self-inactivating (SIN) feature of 3′ LTR enhancer/promoter to inactivate viral promoters upon integration, which allows use of other promoters for regulating expression of the fusion nucleic acid. It is possible to configure these SIN retroviral vectors to permit inducible expression of retroviral inserts after integration of a single vector into a target cell (Hofmann, et al., Proc. Natl. Acad. Sci. USA 93:5185 (1996)). SIN vectors based on Moloney murine leukemia viruses are described in Yu, S-F. et al., Proc. Natl. Aced. Sci USA 83: 3194-3198 (1986); Hwang, J-J. et al., J. Virol. 71: 7128-7131 (1997)). In addition, modifications of the retroviral system by pseudotyping allows a variety of cells types, including non-mammalian cells, to be infected, thus extending the host range of these viral vectors (Morgan, R. A. et al., J. Virol. 67:4712-4721 (1993); Yang, Y. et al., Hum. Gene Ther. 6:1203-1213 (1995); herein incorporated by reference).

Another retroviral vector system useful for introducing and expressing nucleic acids are those based on lentiviruses, such as human immunodeficiency virus (HIV), simian immunodeficiency virus, bovine immunodeficiency virus; feline immunodeficiency virus; and the like (see, e.g., Miyoshi, H. et al., J. Virol. 72: 8150-8157 (1998)). Similar to other retroviral vector, lentiviral vectors employ the viral long terminal repeats (LTR) and Ψ packaging sequence but have the added advantage of infecting both dividing and non-proliferating cells. Lentiviral vectors, including self-inactivating type vectors, and related packaging systems are described in Miyoshi, H., J. Virol. 72:8150-57 (1998); Zufferey, R., J. Virol. 72: 9873-9880 (1998); Iwakuma, T., Virology 261:120-132 (1999); and Xu, K., Mol. Ther. 3: 97-104 (2001); and Mitta, B. et al., Nucleic Acids Res. 30(21):e113 (2002)). The vectors may be packaged into viral particles by transfecting with plasmids encoding the necessary viral genes along with the vector construct (see Kafri, T. et al., Nat. Genet. 17: 314-317 (1997); Naldini, L. et al., Science 272: 263-67 (1996)). In these transient transfection methods, the packaging plasmid constructs express Gag-pol, Tat, Rev, Nef, Vpr, Vpu and Vif proteins while the envelope plasmid constructs express the envelope protein, such as VSV-G, Env of MLV, or GaLV, to serve as the viral envelope. Alternatively, lentivirus packaging cell lines that limit the cytotoxic effects of lentiviral proteins involved in viral packaging are used to generate and propagate the vector (Kafri, T. et al., J. Virol. 73:576-84 (1999)).

Another type of viral vector system for expressing nucleic acids in non-proliferating cells is derived from adenoviruses (see, e.g., Zheng, C. et al., Nat. Biotechnol. 18: 176-180 (2000)). By “adenoviral vectors” herein is meant vectors containing adenoviral nucleic acids sufficient to package the construct into a viral particle and express a nucleic acid sequence cloned into the viral vector. Adenoviral vectors generally exist extrachromosomally, thus limiting the detrimental effects arising from integration into the host chromosome. In one embodiment, the adenoviral vectors containing the fusion nuclei acids of the present invention is made by ligation of the nucleic acid of interest to the adenoviral genome (Rosenfeld, M. A. et al., Science 252:431-434 (1991)). In one embodiment, adenoviral constructs are made by homologous recombination between an adenoviral shuttle vector and a proviral vector in packaging cells that complement replication and packaging of defective adenoviruses (Mittal, S. K. et al. Virus Res. 28:67-90 (1993); Becker, T. C. et al., Methods Cell Biol. 43:161-189(1994)). Alternatively, recombination in bacteria may also be used (He, T-C. et al., Proc. Natl. Aced. Sc. USA 95:2509-2514 (1998). Suitable adenoviral vectors are based on replication defective adenoviruses with deleted early genes (e.g., EIA, E1B, and/or E3 viral genes). Another type of vector constructs, known as helper dependent (HD) adenoviral vectors, are fully deleted of viral gene sequences and contain only the essential cis elements, namely the inverted terminal repeats and packaging signal sequences, required for viral propagation and packaging (Morsey, M. A. et al., Mol. Med. Today 5:18-24 (1999); Parks, R. J. et al., Proc. Natl. Acad. Sci. USA 19:13565-13570 (1996)). Adenoviral vectors have the advantages of infecting a variety of cells types, including proliferating or quiescent cells, and can accommodate larger nucleic acid segments than retroviral vectors. For cell types not readily infected by adenoviruses, the integrin cell surface receptor or adenoviral fiber protein (also known as coxackievirus and adenovirus receptor (CAR)) may be introduced into the cells to increase infection efficiency. Moreover, modifications to the adenovirus fiber protein, for example via insertion of target peptides into the loops of the fiber protein, can increase the range of host cell infectable by adenoviruses (see, e.g., Belousova N. et al., J. Virol. 76:8621-8631 (2002)).

Another type of viral vectors are those based on adeno-associated viruses (MV). Generally, these are small DNA viruses that require coinfection with adenovirus or herpesvirus for efficient replication. In the absence of co-infection, the unmodified MV integrates at high frequency into specific site at chromosomal location 19q13.3, designated as AASV1. AAV constructs containing just the viral inverted terminal repeats can integrate into the host chromosome in a non-specific manner. Integration into the 19q site, however, requires activity of MV rep protein. AAV infects dividing and non-dividing cells and is present episomally in non-dividing cells. Recombinant MV based vectors are generally nonpathogenic, have relatively low immunogenicity, and are capable of long-term transgene expression. When specific integration is desirable, particularly for limiting detrimental effects arising from non-specific integration of the viral nucleic acid into the host chromosome, the AAV vectors contain the rep gene to promote site-specific integration (Owens, R. A., Curr. Gene Ther. 2:145-159 (2002)). Viral particles containing the expression vectors are generated by co-infection of AAV expression vectors with viruses having mutated or deleted terminal repeats or by transfection of the vector into suitable packaging cell lines (Cao, L. et al., Gene Ther. 9:1199-1206 (2002); Qiao, C. P. et al., J. Virol. 76:1904-1913 (2002)). Additionally, cell targeting may be altered by capsid modification, for example by expressing capsid proteins fused to peptide ligands that bind cell surface proteins or by the use of viruses with different serotypes.

In yet a further embodiment, alphaviral vectors are used (Ehrengruber, M. U., Proc. Natl. Acad. Sci. USA 96:7041-7046 (1999)). Alphaviral vectors are made from genome of alphaviruses, which are small, enveloped positive-strand RNA viruses. Examples include Semliki Forest virus (SFV), Sindbis virus (SIN), and Venezuelan equine encephalitis virus. Recombinant viral particles are easily generated, have rapid onset, and generate high-level transgene expression (Koller, D., Nat. Biotechnol. 19:851-855 (2001)). Known vectors are constructed as a replicon, a self-amplifying RNA vector containing the cis and trans alphavirus genetic elements necessary for RNA replication and the native subgenomic promoter for driving expression of heterologous genes. Upon introduction into cells, replicon RNA is translated to express the nonstructural proteins comprising the alphaviral replicase. Replication proceeds through a minus-strand RNA intermediate and subsequently generates two distinct positive-strand RNA species, corresponding to a genomic-length vector RNA and a subgenomic RNA encoding the heterologous gene. The replicon RNA can be packaged into virion-like particles by providing the structural proteins in trans, from in vitro transcribed defective helper RNA or by using appropriate packaging cell lines. Alternatively, the replicon RNA is introduced directly into cells as plasmid DNA. Non-cytopathic versions useful for long-term expression of heterologous nucleic acids are described in Frolov, I., J. Virol. 73:3854-3865 (1999).

Another viral vector useful for Introducing the fusion nucleic acids into cells are based on DNA viruses, including, but not limited to, polyomaviruses, papillomaviruses, and gammaherpesvirus. These vectors propagate episomally, and thus circumvent negative effects arising from integration of viral nucleic acid into a host chromosome. Polyomaviral vectors, particularly those based on BKV, efficiently infects a variety of mammalian cells. Typical polyomaviral vectors contain an origin of replication and T antigen necessary for stable maintenance of the DNA (De Benedetti, A. et al., Nucleic Acids Res. 19:431-436 (1991); Cooper, M. J. Hum. Gene Ther. 4:557-566 (1993)). Papillomaviral vectors, particularly bovinepapilloma (BPV-1) type vectors, also remain episomally within a host cell. Useful vectors contain the entire BPV-1 genome, or comprise deletions up to 31% of the viral genome. Preferably, the vectors contain only the E1, E2 and upstream regulatory regions required for stable replication and maintenance (Ohe, Y. et al., Hum. Gene Ther. 6:325-333 (1995)). Gammaherpesvirinae vectors are made generally from Epstein-Barr virus. In these vectors, stable maintenance of viral plasmid requires only the origin of replication and the trans-acting factor EBNA1. The virus infects a host range that includes human cells, monkey cells, and dog cells (see, e.g., Lei, D. C. et al., Gene Ther. 3:427436), although expression in rodent cells is problematic. However, replacement of origin of replication with sequences mediating replication in rodent cells may overcome this limitation (Krysan, P. J. and Carlos, M. P., Gene 136:137-143 (1993)). A selection gene, such as hygromycin resistance gene, is required for stable propagation since plasmid is slowly lost in the absence of selection pressure. The foregoing expression vectors can provide efficient and sustained expression of the STAT inhibiting agents described herein.

The compositions of the present invention are made according to methods well known and conventional in the art. The nucleic acids and the fusion nucleic acids described above can be prepared using standard recombinant DNA techniques, for example as described in Sambrook, J. et al., Molecular Cloning: A Laboratory Manual, 3rd Ed., Cold Spring Harbor Press, Cold Spring Harbor, N.Y., 2001; Ausubel, F. et al., Current Protocols in Molecular Biology (updates to 2002) Greene Publishing Associates and John Wiley & Sons, New York, N.Y. (1994); the entire contents of which are incorporated by reference. Generally, the expression vectors are made to contain the necessary regulatory or control sequences (e.g., promoters and promoter controlling elements, translation initiation and termination sequences, polyadenylation sequences, splicing signals, etc.), cloning and subcloning sites, reporter/selection or marker genes for identifying cells containing the fusion nucleic acid, priming regions for sequencing and polymerase chain reaction, and the like. As described above, these nucleic acid sequences are operably linked such that the resulting fusion nucleic acids are placed in a functional relationship with each other. That is, the components described are placed in a relationship permitting them to function Is their intended manner.

The nucleic acids encoding STAT inhibiting agents are also made by isolating nucleic acid sequences that hybridize to the nucleic acid sequence of FIG. 2 (SEQ ID NO:2) under conditions in which the isolated nucleic acid is related to the subject nucleic acid by a requisite degree of sequence identity. In one embodiment, the isolated nucleic acids have about 65%, preferably about 80%, more preferably about 90%, and most preferably about 95% or greater sequence identity with the nucleic acid of FIG. 2 (SEQ ID NO:2). The isolated nucleic acid encodes a protein having the characteristics of a STAT inhibiting protein as defined herein. Accordingly, the isolated nucleic acids encode a protein with requisite homology to the amino acid sequence of FIG. 1 (SEQ ID NO:1) and display the various functional characteristics of a STAT inhibiting agent. The hybridization conditions are selected to identify nucleic acid molecules with the requisite degree of homology. Stringent or high stringency conditions comprise a hybridization condition, which is about 10-12° C. below the thermal melting temperature (Tₘ) of the probe being used. For example, a high stringency condition may comprise formamide at 25% to 50% (v/v) In a buffered solution comprising 1× to 6×SSC (1×SSC being 150 mM NaCl and 15 mM sodium citrate; SSPE may replace SSC, where 1×SSPE is 150 mM NaCl, 10 mM Na H₂PO₄, and 1.25 mM EDTA, pH 7.4). The hybridization temperature is typically about 42° C. under the described conditions. High stringency conditions also employ a wash buffer with low ionic strength, such as 0.1× to about 0.5×SSC, at relatively high temperature, typically greater than about 55° C. up to about 70° C. at the described condition, and is generally about 10-12° C. below the Tₘ. Moderately stringent conditions typically use 0% to 25% formamide in 1× to 6×SSC, and use reduced hybridization temperatures, usually in the range of about 27° C. to about 40° C., or about 12-20° C. below the Tₘ. The wash buffer can have increased ionic strength, e.g., about 0.6× to about 2×SSC, and is used at reduced temperatures, usually from about 45° C. to about 55° C. under the described condition. With “non-stringent” or “low stringency” hybridization conditions, the hybridization buffer is the same as that used for moderately stringent or high stringency, but does not contain a denaturing agent. A reduced hybridization temperature is used, typically in the range of about 25° C. to about 30° C., or about 20-30° C. below the Tₘ. The wash buffer has increased ionic strength, usually around 2× to about 6×SSC, and the wash temperature is in the range of about 35° C. to about 47° C. Procedures for nucleic acid hybridizations are well-known in the art (Ausubel et al., supra; Sambrook et al., supra; Shilo and Weinberg, Proc. Natl. Acad. Sci. USA 78:6789-6792 (1981)).

Proteins of the present invention, particularly fragments of STAT inhibiting protein and peptide conjugates for protein transduction, may be prepared in a number of ways. By “protein” as generally used herein refers to at least two covalently attached amino acids, and includes polypeptides, oligopeptides, and peptides. The protein may comprise naturally occurring amino acids and peptide bonds, or synthetic peptide structures. Accordingly, “amino acid” includes both naturally occurring and synthetic amino acids. For instance, homo-phenylalanine, citrulline, and norleucine are considered amino acids as used herein. Amino acids further include imino residues, such as proline and hydroxyproline. Amino acids may have side chains in R or S configuration and be D or L stereoisomer.

Chemical synthesis of proteins and peptides are well known in the art. Solid phase synthesis is commonly used and various commercial synthetic apparatuses are available, for example automated synthesizers by Applied Biosystems Inc., Foster City, Calif.; Beckman Instruments; etc. Solution phase synthetic methods may also be used. General synthetic methods are described in “Solid Phase Peptide Synthesis” in Methods in Enzymology (Fields, G. B. Ed.) Academic Press, San Diego (1997)); Lloyd-Williams, P., Albericio, F. and Giralt, E. Chemical Approaches to the Synthesis of Peptides and Proteins CRC Press, Boca Raton. (1997)). Other references to synthesis of peptides and peptide analogues include, among others, Wipf, P. and Henninger, T. C., J. Org. Chem. 62:1586-1587 (1997); Wellings, D. A. and Atherton, E., “Standard Fmoc protocols,” Methods Enzymol. 289, 44-67 (1997) Walker, M. A., Angew. Chem. Int. Ed. 36, 1069-1071 (1997); Suhara, Y. et al., Tetrahedron Lett. 38:7167-7170 (1997); Songster, M. F. and Barany, G., “Handles for solid-phase peptide synthesis,” in Methods Enzymol., 289,126-174 (1997); Scott, W. L. et al., Tetrahedron Lett., 38, 3695-3698 (1997); O'Donnell, M. J. et al., Tetrahedron Lett. 38:7163-7166 (1997); Muir, T. W. et al., “Protein synthesis by chemical ligation of unprotected peptides in aqueous solution,” in Methods Enzymol. 289:266-298 (1997); Royo, M. et al., Eur. J. Org. Chem. 45-48 (2001)); and Stewart, J. M., “Cleavage methods following Boc-based solid-phase peptide synthesis,” Methods Enzymol. 289:29-44 (1997)).

As will be appreciated by those skilled in the art, segment condensation may be used to synthesize the compositions (Kimura, T. et al., Biopolymers 20:1823-1832 (1981); Sakakibara, S., Biopolymers 37:17-28 (1995); and Canne, L. E. et al., J. Am. Chem. Soc. 121:8720-8727 (1999)). In segment condensation, peptide segments of the final peptide or protein product are synthesized separately and then assembled to form the full length product (see, e.g., Nishuchi, Y. et al., Proc. Natl. Acad. Sci. USA 95:13549-13554 (1998). Depending on the synthetic strategy, solution or solid phase based ligation of the peptide segments is available.

By using these standard techniques, naturally occurring amino acids may be substituted with unnatural amino acids, particularly D-stereoisomers, and also with amino acids with side chains having different lengths or functionalities. Thus, contemplated within the scope of STAT inhibiting agents are proteins and peptides comprising amino acid analogs, including D-amino acids and non-natural amino acid analogs, such as noreleucine, O-methyl-L-tyrosine, 2-aminobutyric acid, α-substituted analogs of α-amino acid, and others known in the art. Functional groups for conjugating to small molecules, label moieties, peptides, or proteins, or for purposes of forming cyclized peptides, may be introduced into the molecule during chemical synthesis. In addition, small molecules and label moieties may be attached during the synthetic process. Preferably, introduction of the functional groups and conjugation to other molecules minimally affects the structure and function of the subject peptide.

For conjugating various molecules to the proteins of the present invention, functional groups on the STAT inhibiting agent and the molecule to be conjugated are reacted in presence of an appropriate conjugating agent. The type of conjugating agent used will depend on the type of functional groups, such as primary amines, sulfhydryls, carbonyls, carbohydrates and carboxylic acids. Conjugating reagents may be fixatives and crosslinking agents, which may be homobifunctional, heterobifunctional, or trifunctional crosslinking agents (Pierce Endogen, Chicago, Ill.). Commonly used fixatives and crosslinking agents include formaldehyde; glutaraldehyde; 1,1-bis(diazoacetyl)-2-phenylethane; N-hydroxisuccinimide esters; dissuccimidyl esters; maleimides (e.g., bis-N-maleimido-1-8-octane); and carbodiimides (e.g., N-ethyl-N′-(3-dimethylaminopropyl)-carbodiimide and dicyclohexylcarbodiimide). Spacer molecules comprising alkyl or substituted alkyl chains with lengths of 2-20 carbons may be used to separate conjugates. Preferably, reactive functional groups on the STAT inhibiting agent not selected for modification are protected prior to coupling of the peptide to other reactive molecules to limit undesired side reactions. By “protecting group” as used herein is a molecule bound to a specific functional group which is selectively removable to reexpose the functional group (see Greene, T. W. and Wuts, P. G. M., Protective Groups in Organic Synthesis, 3rd Ed., John Wiley & Sons, Inc., New York, 1999). The proteins may be synthesized with protected amino acid precursors or reacted with protecting groups following synthesis but before reacting with crosslinking agent. Conjugations may also be indirect, for example by attaching a biotin moiety, which can be contacted with a compound or molecule coupled to streptavidin or avidin.

For STAT inhibiting agents that have reduced activity in the conjugated form, the linkage between the agent and the conjugated compound is chosen to be sufficiently labile to result in cleavage under desired conditions, for example after transport to desired cells or tissues. Biologically labile covalent bonds, e.g., imimo bonds and esters, are well known in the art (see U.S. Pat. No. 5,108,921). These modifications permit administration of the STAT inhibiting proteins in a potentially less active form, which is then activated by cleavage of the labile bond.

In the present invention, the STAT inhibiting proteins may be purified or isolated after synthesis or expression. By “purified” or “isolated” is meant substantially free of cellular material or free of chemical precursors or other chemicals used from the environment in which the peptide is synthesized or expressed and in a form where it can be practically used. The level of purification will depend on the intended use. Generally, the substantially free compositions used will comprise at least about 20% by weight of the desired product (by weight), more usually at least about 30% by weight, preferably at least about 50% by weight, more preferably at least about 70% by weight, and particularly at least about 80% by weight in relation to the contaminants present. The protein and derivatives of the compositions thereof may be purified and isolated by way known to those skilled In the art, depending on other components present in the sample. Standard purification methods include electrophoretic, immunological, and chromatographic techniques, including ion exchange, hydrophobic, affinity, size exclusion, reverse phase HPLC, and chromatofocusing. The proteins may also be purified by selective solubility, for instance in the presence of salts or organic solvents. The degree of purification necessary will vary depending on use of the subject STAT inhibiting agents. Thus, in some instances no purification will be necessary.

The peptide may also be made in the form of a salt, generally in a salt form that is pharmaceutically acceptable. These include inorganic salts of sodium, potassium, lithium, ammonium, calcium, magnesium, iron, zinc, copper, manganese, and the like. Various organic salts of the peptide may also be made with, Including, but not limited to, acetic acid, propionic acid, pyruvic acid, maleic acid, succinic acid, tartaric acid, citric acid, benozic acid, cinnamic acid, salicylic acid, etc.

Synthesis of the STAT inhibiting agents, and derivatives thereof, may also be carried out by using recombinant techniques. For recombinant production, the protein is expressed from a nucleic acid sequence encoding the STAT inhibitory agent. Generally, a nucleic acid encoding the proteins of the present invention is joined to DNA sequences that permit expression of the nucleic acid and the encoded protein introduced into an appropriate host cell for expression. Expression of the fusion nucleic acid is under the control of a suitable promoter and other control sequences, as described above, for expression in the particular host cell or organism (see, Sambrook, J. et al., supra; Ausubel, F. et al., supra)

Once made, the proteins and nucleic acids made according to methods described herein are used in methods for inhibiting STAT activity. As summarized above, STAT proteins—signal transducers and activators of transcription—comprise a family of transcription factors having a number of structurally and functionally shared features. The amino terminal is conserved between STATs and is believed to be involved in cooperative Interactions in DNA binding and regulating nuclear localization. The coiled-coil domain, from about residues 135-315, interacts with regulatory proteins, such as IRF-9 and StlPI. The DNA binding domain located at the carboxy terminal region recognizes and binds to GAS family of enhancer sequences having the general sequence TTN₅₋₆AA. STAT2 does not bind DNA by itself but associates with STAT1A to form a heterodimeric complex. The src homology 2 (SH2) domain, highly conserved between STATs, functions in receptor interactions and STAT dimerization. The carboxy terminus also contains the transcriptional activation domain (TAD). Isoforms of STAT1, STAT3 and STAT4 generated by alternative splicing have unique carboxy terminal sequences.

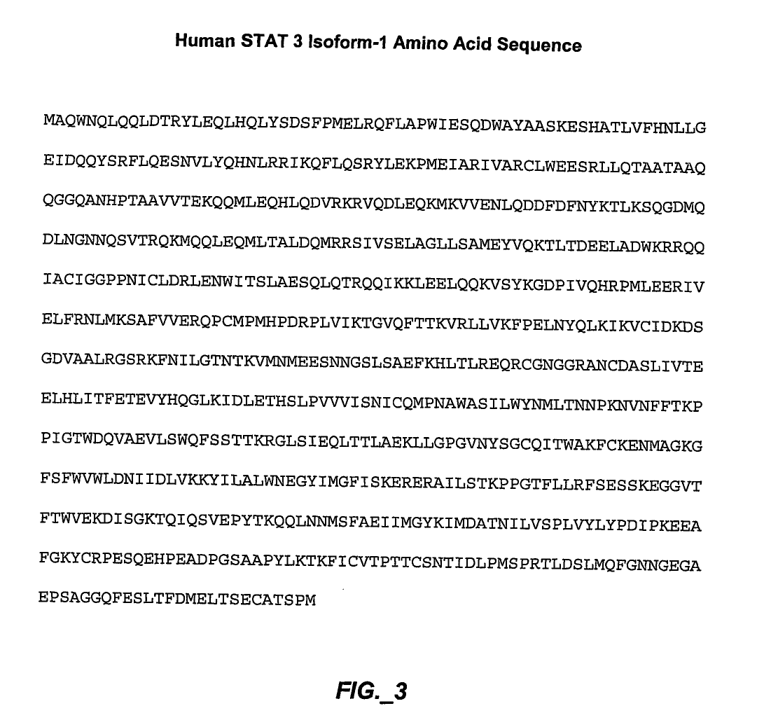

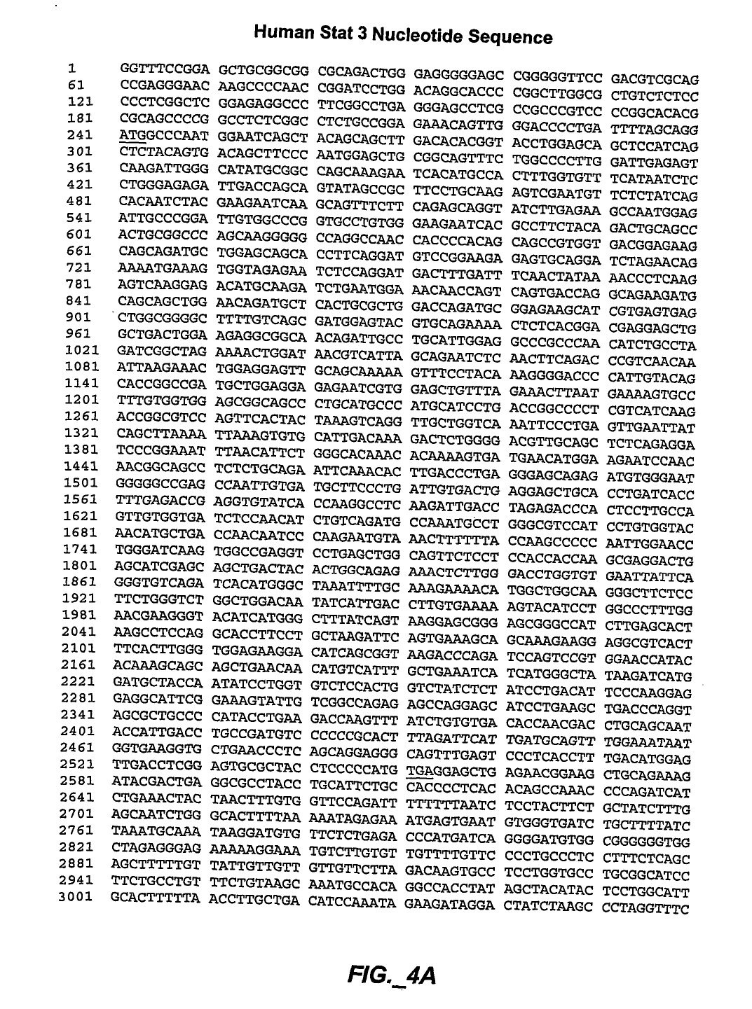

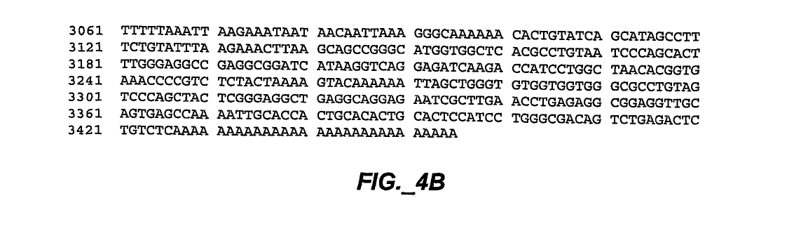

In the present invention, the compositions of the present invention inhibit STAT activity, particularly activity of STAT3 and/or STAT 1, and particularly the activity of STAT3. As used herein, “STAT3 protein” or “STAT3” comprises the amino acid sequence in FIG. 3 and related proteins having a homologous sequence, as discussed above. STAT3 protein is activated in response to cytokines, such as IL6, LIF and CNTF and is necessary for epithelial cell apoptosis, downregulation of inflammatory cytokines, macrophage inactivation, skin development, and keratinocyte migration. STAT3 interacts with Jun protein to induce transcription of a-2-macroglobulin gene. An isoform of STAT3, denoted STAT3β, generated by alternative splicing, has a truncated carboxy terminus. This shortened form is encompassed within the definition of STAT3 (Schaefer, T. S. et al., Proc. Natl. Acad. Sci. USA 92:9097-9101 (1995)), as are other synthetic and natural variants of STAT3 having the associated activities of STAT3 protein.

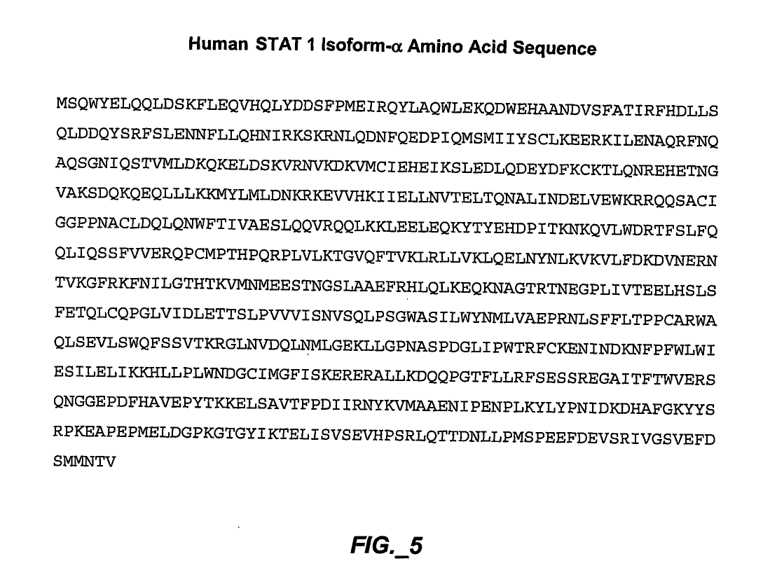

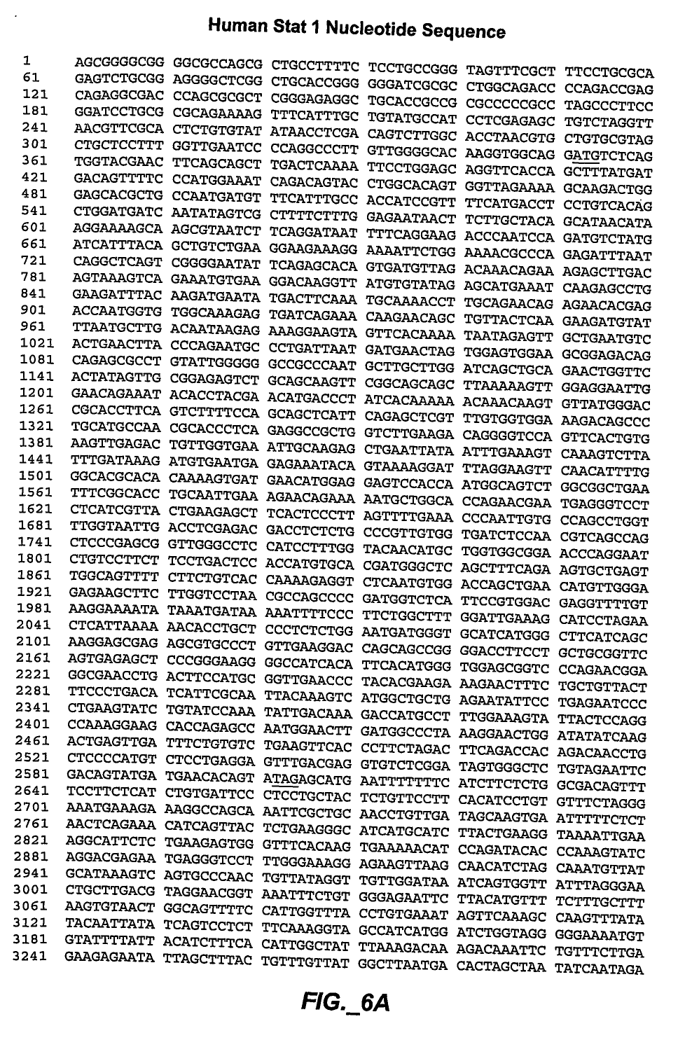

Similar to STAT3, STAT1 exists as full length STAT1a and a shorter STAT1β generated by alternative splicing. The shorter isoform Is missing the carboxy terminal 38 amino acids. As used herein, STAT1 protein comprises the amino acid sequence in FIG. 5 and the isoforms generated by alternative splicing. As above, encompassed within STAT1 are synthetic and natural variants having an amino acid sequence homologous to the amino acid sequence in FIG. 5 and having the associated activities of STAT1 protein. STAT1 is activated by interferons IFNα and IFNγ, with the latter requiring STAT1α for full activity. STAT1 is known to associate with a number of proteins, including, the CREB binding protein/p300 related group of histone acetyl transferases; Nmi-1, which is an N-myc-interacting protein; MCM5 protein involved in DNA replication; breast and ovarian cancer susceptibility protein, BRCA1; and interferon response factor IRF p48.

The present invention provides for various methods of inhibiting STAT3 and STAT1 activities. In one embodiment, the method comprises contacting a system containing STAT3 or STAT1 with a sufficient amount of STAT inhibiting agents described herein, whereby activity of the STAT protein is inhibited or attenuated. In one aspect, the system comprises a cell free system containing the components necessary for complex formation between a STAT protein and STAT inhibiting agent; ubiquitinylation of the associated STAT protein; and preferably, subsequent degradation of the ubiquitin conjugated molecule by the ubiquitin proteasome pathway. Cell free systems are described in Nguyen H., Mol. Cell Biol. 19:1190-1201 (1999) and DeMartino, G. N., Biochim. Biophys. Acta 1073:299-308 (1991) while systems with purified components described in Shkedy, D., FEBS Lett. 348:126-30 (1994). STAT inhibiting agent added to the system may comprise full-length protein or a protein fragment (e.g., peptide). For a cell free system containing components sufficient for translation of RNA, an RNA molecule encoding the STAT inhibiting agents may be added.

Cell free systems are useful in assays or screens for other STAT inhibiting agents with similar functional activity as the protein of FIG. 1 (SEQ ID NO:1), including fragments of the agents described herein. Furthermore, cell free systems provide a method of identifying factors necessary for STAT inhibiting activity and are useful in screens for enhancers or antagonists of STAT inhibiting agents. Candidate agents may comprise naturally occurring proteins or fragments thereof. These may be found in cellular extracts or as part of digests of proteinacious extracts. Candidate peptide agents also encompass random peptides, synthesized by chemical methods or expressed from nucleic acids of random sequences. The synthetic peptides are of from about 5 to about 30 amino acids, with from about 5 to about 20 amino acids being preferred, and from about 7 to about 15 amino acids being particularly preferred.

In other embodiments, the candidate agents are small organic compounds, preferably having a molecular weight of more than 100 and less than about 2,500 daltons. Candidate agents of this nature often comprise cyclical carbon or heterocyclic structures, and/or aromatic or polyaromatic structures. Libraries of synthetic or natural compounds may be obtained from a variety of sources. Random and directed syntheses of organic compounds are well known In the art (Gallop, M. A., J. Med. Chem. 37:1233-1251 (1994); Gordon, E. M. et al., J. Med. Chem. 37:1385-1401 (1994); Thompson, L. A. et al., Chem. Rev. 96:555-600 (1996); Balkenhol, F. et al. Angew Chem Int Ed. 35:2288-2337 (1996); and Gordon, E. M. et al., Acc. Chem. Res. 29:444-454 (1996)). Alternatively, libraries of natural compounds in the form of bacterial, fungal, plant, and animal extracts are readily available or produced. Additionally, natural or synthetically produced compounds are modifiable by conventional chemical, physical or biochemical means. For instance, known pharmacological agents may be subjected to directed or random chemical modifications such as acylation, alkylation, esterification, and amidification to produce structural analogs.

In one embodiment, the compositions of the present invention are used in a method for inhibiting STAT activity in a cell. Generally, the method comprises contacting at least one cell with a compound comprising a STAT inhibiting agent in an amount effective to attenuate, inhibit, or decrease activity of the STAT protein, preferably STAT3 and STAT1 protein, and particularly the STAT3 protein. The compound comprises a STAT inhibiting protein as defined above or alternatively, a nucleic acid encoding the STAT inhibiting protein. STAT inhibiting agent is present within the cells to be treated, and in some embodiments, produced by the cells, for instance via an expression vector encoding the STAT inhibiting agent. Inducing expression of the STAT inhibiting agent in the cell is sufficient to contact the cell with the inhibiting agent. Treatment of cells is accomplished in vitro, ex vivo or in vivo. For in vivo applications, the host or subject may be any mammal including domestic animals, pets, laboratory animals, primates, and particularly human subjects.

In another embodiment, the agents are useful for inhibiting or attenuating the expression of genes regulated by STAT-3 or STAT-1. Expression of target genes is analyzable in cell free systems, or preferably, in intact cells. A cell or population of cells is contacted as described above with a STAT inhibiting agent in an amount sufficient to attenuate, inhibit, or decrease expression of target genes regulated by STATs, preferably STAT3 or STAT1, particularly STAT3, which act via binding to GAS sequences in the target genes. Specific target genes whose expression is regulated by STAT3 include, but are not limited to, anti-apoptotic protein BCL-xl; anti-apoptotic protein Mcl-1; oncogene c-Myc; cyclin D1; protooncogene JunB, serum amyloid A (SM3), Janus kinase inhibitor JAB (e.g., SOCS1/SSI1), C-reactive protein, and cell cycle inhibitor p21. Genes identified as being regulated by STAT1 include, but are not limited to, interferon stimulated gene ISG54; interferon regulatory factor IRF-1; MHC class II transactivator CIITA; inducible nitrous oxide synthase (iNOS); monokine induced by IFNγ (MIG), 1,3 dioxygenese, GBP, and cell cylce inhibitor p21. In some embodiments, expression of target genes may be increased, for instance by inhibiting expression of a target gene that inhibits expression of other genes. Assays for expression of target genes include use of gene product specific antibodies (e.g., immunoprecipitation, Western analysis, etc.), reporter gene expression based on operable linkage of cognate promoter and a reporter gene; measuring RNA synthesis (e.g., Northern analysis, primer extensions, gene chip assays, etc.); and others known In the art.

In yet another embodiment, the agents may be used for inhibiting signaling by various ligands, such as cytokines and growth factors. Generally, cells are contacted with a STAT inhibiting agent in an amount sufficient to attenuate, inhibit, or decrease cellular effects of the cytokine or growth factor. Treatment of cells with the ligand may be prior or subsequent to contacting cells with STAT inhibiting agent. In some embodiments, the cells to be treated express the STAT inhibiting agent via an expression vector contained within the cell. Ligands exerting its effects via activity of STAT-3 or STAT-1 include, but are not limited to, IFNs (IFNα and IFNγ), EGF, IL-5, IL6, HGF, LIF, BMP-2, and CD8⁺ T-lymphocyte antiviral factor (CAF). Growth factors include, but are not limited to, PDGF, EGF, and NRG-1. Cellular effects of a cytokine or growth factor are measured in a variety of ways well known in the art. These include measuring morphological changes to cells; alterations in proliferate capacity of cells; expression of various target genes regulated by the cytokine or growth factor; changes in modification of cellular components (e.g., phosphorylation); alterations in cellular localization of cellular components (e.g., nuclear localization of STAT protein); and the like.

Various cells types are amenable to use with the STAT inhibiting agents. These include, among others, yeast such as Saccharomyces cerevisiae and Saccharomyces phoebe, insects and insect cells (e.g., Drosophila, Schneider cells, KC cells); amphibian cells (e.g., Xenopus), avian cells, and particularly mammalian cells, most preferably cells expressing STAT3 or STAT1, including cells recombinantly engineered to express STAT3 or STAT1. Mammalian cell types suitable as targets for STAT inhibiting agents include, without limitation, rodent, primate, and human cells. Particularly preferred are cancer cells in which STAT1 or STAT3 activity, particularly STAT3, is elevated or constitutively active, including, among others, multiple myeloma; leukemia, Including HTLV-1 dependent, chronic lymphocytic leukemia, acute myelogenous leukemia, large granular lymphocyte leukemia; lymphomas, including EBV related Burkitt's, mycosis fungoides; HSV saimiri-dependent (T-cell); cutaneous T-cell lymphoma; and solid tumors, including breast cancer, squamous cell carcinoma of the head and neck (SCCHN), renal cell carcinoma, melanoma, ovarian carcinoma, lung carcinoma, prostate carcinoma, and pancreatic adenocarcinoma. Other cells types appropriate for use with the STAT inhibiting agents include cardiomyocytes, endothelial cells, epithelial cells, hepatocytes, leukocytes (e.g., mononuclear leukocytes), stem cells (e.g., hematopoetic, neural, skin, lung, kidney, liver, and myocyte), osteoclasts, chondrocytes, keratinocytes, kidney cells, and adipocytes.

Varieties of techniques are available for introducing proteins and nucleic acids into cells. By “introduced” into herein is meant that protein is delivered into the cell or that a nucleic acid enters the cells in a manner suitable for subsequent expression of the nucleic acid. Technique used for delivery into cells will vary depending on the nature of the composition and whether cells are in vitro, ex vivo, or in vivo, and the type of cell or host organism. When cells are treated ex vivo, the cells may be autologous cells, which are cells obtained from the animal prior to reintroduction into the same organism. Exemplary techniques for introducing proteins and nucleic acids into cells include the use of liposomes, Lipofectin®, electroporation (in vivo and in vitro), microinjection, cell fusion, DEAE dextran, calcium phosphate precipitation, viral vectors, and biolistic particle bombardment. Those skilled in the art can choose the method appropriate for the particular application.

In one aspect, the proteins and peptides are conjugated to small organic molecules to facilitate cellular uptake. Typically, the small organic molecules act as ligands for cellular receptors or are substrates for membrane transport proteins. Examples of small organic molecules that promote cellular entry of attached compounds include, without limitation, lipids; carbohydrates and their derivatives, such as β-glycoside; steroids (bile acids, cholesterol, digoxigenin,; biotin; and the like, as described herein.

In another aspect, STAT inhibiting proteins are expressed as fusions or conjugated to peptide sequences that promote entry of the inhibiting proteins into cells. In one embodiment, the peptide sequences are protein transduction sequences described above, such as VP22 protein, HIV Tat protein, arginine rich peptides, Anntennapedia homeobox; peptide delivery agents (see, e.g., Morris, M. C. et al., Nucleic Acids Res. 25: 2730-2736 (1997); amphipathic peptide carriers (Morris, M. C. et al., Nat. Biotechnol. 19:1173-1176 (2001); Syn B vectors, which are derivatives of antimicrobial peptide protegrin (see, e.g., Dorn, G. W. I. et al., Proc. Natl. Aced. Sci. USA 96:12798-12803 (1999); Schwartz, S. R. et al., Science 285:1569-1572 (1999). In another embodiment, the STAT inhibiting agents are conjugated to antibodies, particularly monoclonal antibodies directed to cell surface receptors or cell surface components. In a further embodiment, conjugation is to peptide ligands bound by receptors, including, but not limited to cytokines, chemokines, and growth factors. In these delivery systems, the antibodies or ligand may be attached directly to the protein or nucleic acid, or attached indirectly. An example of indirect linkage is covalently attaching biotin to the antibody or ligand and reacting with a STAT inhibiting agent having an attached streptavidin molecule.

Attachment to bacterial toxin carriers provides another method for introducing STAT inhibiting agent into cells, Suitable toxins include, but are not limited to, diptheria toxin (DT), cholera toxin (CT), B-subunit of E. coli. heat-labile enterotoxin, and Pseudomonas exotoxin. The conjugates are taken up into the endocytic pathway of eukaryotic cells, and the peptides liberated and translocated into the cytosolic and/or nuclear compartment of the cell, thus providing a method of introducing the subject compositions into the cells (Loregian A et al., Proc. Natl. Aced. Sci. USA 96, 5221-5226 (1999)).

For compositions comprising nucleic acids encoding the STAT inhibiting proteins, delivery into cells is accomplished by the methods discussed above. For example, nucleic acids encoding the STAT inhibiting agents are introduced into cells by viral vectors. As described above, these include viral particles in the form of retroviruses, lentiviruses, adenoviruses, adeno-associated virus, polyomaviruses, papillomaviruses, and other viral transducing systems known in the art. Typically, the viral vectors encoding the inhibiting agents are introduced into a suitable packaging cell line or co-transfected with a defective virus to generate viral particles containing the vector. The viral particles are harvested, concentrated, and used to infect target cells. Viral delivery systems are chosen so as not to interfere with the STAT inhibiting agents described herein, although in some instances, viruses may be chosen to increase the types of STAT proteins inhibited by the treatment. Viral vector systems are applicable for both in vitro and in vivo delivery.

Alternatively, the STAT inhibiting proteins or the nucleic acids are introduced into cells via non-viral systems. For in vivo delivery, a non-viral system may use naked DNA administered into tissue or blood. The DNA is taken up by the cells or organ with subsequent expression of the encoded protein product (Shi, F. et al., Mol. Cancer Ther. 1 (11):949-957 (2002); Cui, F. D. et al., Gene Ther. 8(19):1508-1513 (2001)). Delivery is by catheter or direct injection into the tissue or organ being targeted. Additives or other compounds may be co-administered to enhance uptake. For example, addition of chlorpromazine and related phenothiazines is shown to enhance uptake of nucleic acids into cells. Moreover, use of electroporation techniques, typically involving brief electrical pulses applied at the site of DNA injection, enhances uptake of the naked nucleic acid by the surrounding cells in vivo.

Another useful non-viral delivery system comprises encapsulation of the subject compounds in liposomes. By “liposomes” herein is meant the art recognized synthetic particles composed of lipids, particularly phospholipids. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids, such as cholesterol; cerebrosides; sphingomyelin; glycerolipids; and other lipids (see for example, U.S. Pat. No. 5,833,948).

Cationic lipids are also suitable for forming liposomes. Generally, the cationic lipids have an net positive charge and have a lipophilic portion, such as a sterol, an acyl or diacyl side chain. Preferably, the head group is positively charged. Cationic lipids interact with negatively charged compounds, such as a DNA molecule, to form clusters or aggregated vesicles, and at a certain density, condenses to encapsulate the negatively charged compound within the lipid bilayer. Typical cationic lipids include 1,2-dioleyloxy-3-(trimethylamino)propane; N-[1-(2,3,-ditetradecycloxy)propyl]-N,N-dimethyl-N—N-hydroxyethylammonium bromide; N-[1-(2,3-dioleyloxy)propyl]-N,N-dimethyl-N-hydroxy ethylammonium bromide; N-[1-(2,3-dioleyloxy) propyl]-N,N,N-trimethylammonium chloride; N-[1-(2,3-dioleyloxy) propyl]-N,N,N-trimethylammoniumethyl sulphate; 3-[N—(N′,N′-dimethylaminoethane) carbamoyl] cholesterol; and dimethyldioctadecylammonium. Use of cationic thiol-detergent, tetradecyl-cysteineyl-ornithine generates lipids coating a nucleic acid of interest about the size of the nucleic acid itself, resulting in efficient distribution and uptake by cells. Generally, the cationic liposomes include a neutral lipid to enhance membrane fusion.

Of particular interest are fusogenic liposomes, which are characterized by their ability to fuse with a cell membrane upon appropriate change in physiological condition or by presence of fusogenic component, particularly a fusogenic peptide or protein. In one aspect, the fusogenic liposomes are pH and temperature sensitive in that fusion with a cell membrane is affected by change in temperature and/or pH (see, e.g., U.S. Pat. Nos. 4,789,633 and 4,873,089). Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following liposome endocytosis (see, e.g., Mizoue, T., Int. J. Pharm. 237: 129-137 (2002)).

Another form of fusogenic liposomes comprises liposomes that contain a fusion enhancing agent. That is, when incorporated into the liposome or attached to the lipids, the agents enhance fusion of the liposome with other cellular membranes, thus resulting in delivery of the liposome contents into the cell. The agents may be fusion enhancing peptides or proteins, including hemaggulutinin HA2 of influenza virus (Schoen, P., Gene Ther. 6: 823-832 (1999)); Sendai virus envelope glycoproteins (Mizuguchi, H., Biochem. Biophys. Res. Commun. 218:402407 (1996)); vesicular stomatitis virus envelope glycoproteins (VSV-G) glycoprotein (Abe, A. et al., J. Virol. 72: 6159-63 (1998)); peptide segments or mimics of fusion enhancing proteins; adhesive peptides which promote binding of liposomes to specific cells; and synthetic fusion enhancing peptides, as described herein (see also Kono, K. et al., Biochim. Biophys. Acta. 1164: 81-90 (1993); Pecheur, E. I., Biochemistry 37: 2361-71 (1998); U.S. Pat. No. 6,372,720).

Liposomes also include vesicles derivatized with a hydrophilic polymer, as provided in U.S. Pat. Nos. 5,013,556 and 5,395,619, hereby Incorporated by reference, (see also, Kono, K. et al., J. Controlled Release 68:225-235 (2000); Zalipsky, S. et al., Bioconjug. Chem. 6:705-708 (1995)) to extend the circulation lifetime in vivo. Hydrophilic polymers for coating or derivation of the liposomes include polyethylene glycol (PEG), polyvinylpyrrolidone, polyvinylmethyl ether, polyaspartamide, hydroxymethyl cellulose, hydroxyethyl cellulose, polylysine, and the like. PEG stabilized lipid particles encapsulating the protein or nucleic acid generates particles having rapid circulation and uptake in vivo (Mok, K. W. et al., Biochem. Biophys. Acta 1419:137-150 (1999)). Preferred are pH sensitive PEG-lipid conjugates, such as ortho ester PEG-lipid, which rapidly releases the liposome contents into the cytoplasm following internalization in endosomes (Guo, X. and Szoka, F. C., Bioconjug. Chem. 12:291-300 (2001)). Incorporation of polycations, such as polysine, in the liposomes generates liposome polycation complexed to negatively charged molecules (e.g., DNA) which display enhanced delivery into cells. In addition, as described above, attaching proteins that bind a cell surface protein which is endocytosed, e.g., capsid proteins or fragments thereof tropic for a particular cell types and antibodies for cell surface proteins which undergo internalization, may be used for targeting and/or facilitating uptake of the liposomes to specific cells or tissues. Alternatively, fusogenic peptides or protein transduction peptides may be used.

Liposomes are prepared by ways well known in the art (see, e.g., Szoka, F. et al., Ann. Rev. Biophys. Bioeng. 9:467-508 (1980)). One typical method is the lipid film hydration technique in which lipid components are mixed in an organic solvent followed by evaporation of the solvent to generate a lipid film. Hydration of the film in aqueous buffer solution, preferably containing the subject peptide or nucleic acid, results in an emulsion, which Is sonicated or extruded to reduce the size and polydispersity. Other methods include reverse-phase evaporation (Pidgeon, C. et al., Biochemistry 26:17-29 (1987); Duzgunes, N. et al., Biochim. Biophys. Acta. 732:289-299 (1983)), freezing and thawing of phospholipid mixtures, and ether infusion.

Similar to liposomes, reconstituted viral envelopes or virosomes serves as delivery vehicles for introducing proteins, nucleic acids, and drugs into cells (see, e.g., Schneider J., J. Gen. Virol. 64:559-65 (1983)). Generally, isolated viruses are solubilized in the presence of detergent, such as Triton X-100 or octa(ethylene glycol)-mono(ndodecyl)ether, and reconstituted in the presence of various types of lipids (see, e.g., Stegman, T. et al., EMBO J. 6:2651-2659 (1987) or by detergent removal (e.g., by use of SM2 bio beads, Bio Rad). Inclusion of a protein of interest or a nucleic acid during vesicle reconstitution results in their incorporation into the virosome. Large aggregates are removed by appropriate filtration and/or centrifugation. Reconstituted virosomes are available for a number of viruses, including, among others, Sendai virus, influenza virus, and vesicular stomatitis virus. When viruses containing neuramimidase are used, such as Sendai and influenza viruses, the neuramimidase may be depleted or replaced to provide more targeted delivery to cells. Virosomes lacking neuramimidase bypasses the lysosomal processing which occurs for neuramimidase containing virosomes, thus resulting in direct delivery to the cytoplasm (Bagai, S. and Sarkar, D. P. et al., J. Biol. Chem. 21:1966-1972 (1994)). Alternatively, polyethylene derivatized lipids are incorporated in the virosome to limit preferential interaction of neuramimidase with sialic acid containing components on the cell membrane (see, e.g., Mastrobattista, E. et al., FEBS Lett. 509:71-76 (2001)). In addition, cell specific ligands, antibodies (e.g., monoclonal anitbodies), peptides (e.g., fusogenic peptides), or lipid moieties that bind cell surface receptors are introduced into the virosome to target it to a desired cell type. Examples of specific targeting molecules include Fab′ fragments of anti-rNeu monoclonal antibody, P0 protein, and asialoglycoprotein.

Another type of drug delivery method is erythrocytes modified to encapsulate macromolecules. Erythrocytes may be loaded by hypotonic preswelling in the presence of the macromolecule to be delivered and the cells re-infused into the same or an immunocompetent compatible recipient. Drug-loaded erythrocytes can also be modified to be selectively recognized by certain cell types, such as macrophages (Magnani, M. et al., Gene Ther. 9:749-751 (2002)).

In accordance with the above, the present invention provides for various methods of using the STAT inhibiting agents for the therapeutic benefit in treating various diseases and disorders. In view of the role of STAT3 and STAT1 proteins in mediating effects of various cytokines and growth factors, disorders or diseases amenable to treatment with the STAT inhibiting agents include, but are not limited to, inhibiting tumour cell growth, allergic responses, inflammatory disorders, autoimmune diseases, neurodegenerative disorders, and fibrotic disease. Thus, any disorder or disease in which inhibition of STAT3 or STAT1, particularly STAT3, will ameliorate the condition may be treated with the STAT inhibiting agents.

In one embodiment, the STAT inhibiting agents are used in methods to inhibit growth of tumor cells. In one embodiment, a pharmaceutically effective amount of the STAT inhibiting agents, or preparations thereof, are administered to a subject via an appropriate route to inhibit tumor cell growth or tumor progression. The methods are applicable to tumor cells where STAT3 or STAT1 activity, particularly STAT3 activity, is elevated or constitutively active (Buettner, R., Clin. Cancer Res. 8:945-954 (2002)). Tumors in which STAT3 activation participates in tumor development and progression, include, among others, multiple myeloma; leukemia, including HTLV-1 dependent, chronic lymphocytic leukemia, acute myelogenous leukemia, large granular lymphocyte leukemia; lymphomas, including EBV related Burkitt's, mycosis fungoides; HSV saimiri-dependent (T-cell); cutaneous T-cell lymphoma, Hodgkins disease; and solid tumors, including breast cancer, SCCHN, renal cell carcinoma, melanoma, ovarian carcinoma, lung cancer, prostate carcinoma, brain cancer, Kaposi's sarcoma, and pancreatic adenocarcinoma. In a further embodiment, the subject compositions are used to inhibit tumors arising from dysregulation of src protein kinase since STAT inhibiting agents inhibit signaling via the src kinase activity.

In inhibiting tumor cell growth, the compositions of the present invention may be used in combination of other chemotherapeutic agents. These include, among others, DNA damaging agents (e.g., bleomycin, cisplatin, daunorubicin, doxorubicin, etoposide, cyclophosphamide, mitomycin, etc.); antimetabolites (e.g., methotrexate, mercaptopurine, fluorouracil, and hydroxyurea); mitotic spindle inhibitors (e.g., vinblastine, vincristine, and pacitaxel); immunotherapeutics (e.g., herceptin); and the like. In addition, combination treatments may use other inhibitors of STAT proteins, including agents that disrupt STAT recruitment to a receptor; inhibit phosphorylation of SH2 domains; promote STAT dephosphorylation; inhibit STAT dimerization; disrupt nuclear translocation, interfere with DNA binding and transcriptional activation; and reduce or Inhibit STAT protein expression. In a further embodiment, the subject inhibitors are combined with anti-viral agents such as e.g., ribavirin and interferon.

In another embodiment, the compositions are useful in modulating the immune response. This derives from the role of STAT proteins in regulating responses to various cytokines. In one embodiment, a pharmaceutically effective amount of STAT inhibiting agents is administered to a subject to treat inflammatory reactions associated with a number of disease conditions and tissue injury, particularly in conditions associated with elevated or hyperactive levels of STAT3. These include, but are not limited to, inflammatory bowel disease; Crohn's disease; multiple sclerosis; ischemia; stroke; traumatic brain injury; spinal injury; pulmonary fibrosis, rheumatoid arthritis; atherosclerosis; and acute phase response (Acarin, L. et al., Neuroreport 24; 9(12):2869-73 (1998); Suzuki, A. et al., J. Exp. Med. 193(4):471-81 (2001)).

In a further embodiment, the agents are useful in treating autoimmune diseases. Various autoimmune diseases treatable with a pharmaceutically effective amount of STAT inhibiting agents are, but not limited to, insulin-dependent diabetes mellitus, systemic lupus erythematosus, myasthenia gravis, scleroderma, and psoriasis. In particular, the methods of the present invention are useful in treating inflammatory conditions with elevated STAT3 activity (Wang, F. et al., J. Exp. Med. 182(6):1825-31 (1995). In particular, autoimmune diseases, autoimmune related inflammation, or immune dysregulation arising from IL6 and IL10 mediated activation, including activation by homologs (e.g., IL-10 homolog IL-20), are well suited for treatment with the STAT inhibiting agents, especially where IL6 or IL10 acts in an autocrine mode to affect disease initiation or progression (Miki, S. et al., FEBS 250:607-610 (1989); Blumberg, H. et al., Cell 104(1):9-19 (2001). For example, patients with lupus erythremetosus show elevated levels of cytokine IL-10, which is known to activate STAT3 mediated signaling events (Ripley, Be J. et al., Immunology 97(2):226-31 (1999)).

Other disorders within the ambit of the present invention are, among others, neural disorders, particularly neurodegenerative disorders, for example, Parkinson's disease and Alzheimer's disease; pulmonary fibrosis; and adult respiratory distress syndrome.

In accordance with the above, the present invention further provides for administering a pharmaceutical composition comprising a pharmaceutically acceptable carrier and a pharmacologically effective amount of one or more of the STAT inhibiting agents or nucleic acids encoding the subject agents. The pharmaceutical composition may be formulated as powders, granules, solutions, suspensions, aerosols, solids, pills, tablets, capsules, gels, topical crimes, suppositories, transdermal patches, etc.

Pharmaceutically acceptable salts of the peptides are intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts include organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any of standard pharmaceutically accepted carriers known to those of ordinary skill in the art in formulating pharmaceutical compositions. Thus, the STAT inhibiting proteins, by themselves, such as being present as pharmaceutically acceptable salts, or as conjugates, or nucleic acid vehicles encoding such proteins, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients. Additionally, the formulations may include bactericidal agents, stabilizers, buffers, emulsifiers, preservatives, sweetening agents, lubricants, or the like. If administration is by oral route, the inhibiting agents may be protected from degradation by using a suitable enteric coating, or by other suitable protective means, for example internment in a polymer matrix such as microparticles or pH sensitive hydrogels.

Suitable pharmaceutical formulations may be found in, among others, Remington's Pharmaceutical Sciences, 17th Ed., Mack Publishing Co., Philadelphia, Pa., 1985 and Handbook of Pharmaceutical Excipients, 3rd Ed, Kibbe, A. H. ed., Washington D.C., American Pharmaceutical Association, 2000; hereby incorporated by reference in their entirety. The pharmaceutical compositions described herein can be made in a manner well known to those skilled in the art by means conventional in the art, including mixing, dissolving, granulating, levigating, emulsifying, encapsulating, entrapping, or lyophilizing processes.

In another embodiment, the carriers are in the form of microparticles, microcapsules, microspheres and nanoparticles, which may be biodegradable or non-biodegradable (see for example, Microencapsulates: Methods and Industrial Applications, Drugs and Pharmaceutical Sciences, Vol 73, Benita, S. ed, Marcel Dekker Inc., New York, 1996; incorporated by reference). This formulation is particularly useful for delivery of short peptide agents. As used herein, microparticles, microspheres, microcapsules and nanoparticles mean a particle, which is typically a solid, containing the substance to be delivered. The substance is within the core of the particle or attached to the particle's polymer network. Generally, the difference between microparticles (or microcapsules or microspheres) and nanoparticles is one of size. As used herein, microparticles have a particle size range of about 1 to about >1000 microns. Nanoparticles have a particle size range of about 10 to about 1000 nm.

A variety of materials are useful for making microparticles. Non-biodegradable microcapsules and microparticles include, but not limited to, those made of polysulfones, poly(acrylonitrile-co-vinyl chloride), ethylene-vinyl acetate, hydroxyethylmethacrylate-methyl-methacrylate copolymers. These are useful for implantation purposes where the encapsulated compositions diffuse out from the capsules. In another aspect, the microcapsules and microparticles are based on biodegradable polymers, preferably those that display low toxicity and are well tolerated by the immune system. These include protein based microcapsulates and microparticles made from fibrin, casein, serum albumin, collagen, gelatin, lecithin, chitosan, alginate or poly-amino acids such as poly-lysine. Biodegradable synthetic polymers for encapsulating may comprise polymers such as polylactide (PLA), polyglycolide (PGA), poly(lactide-co-glycolide) (PLGA), poly(caprolactone), polydioxanone trimethylene carbonate, polyhybroxyalkonates (e.g., poly(β-hydroxybutyrate)), poly(γ-ethyl glutamate), poly(DTH iminocarbony (bisphenol A iminocarbonate), poly (ortho ester), and polycyanoacrylate. Various methods for making microparticles containing the subject compositions are well known in the art, including solvent removal process (see, e.g., U.S. Pat. No. 4,389,330); emulsification and evaporation (Maysinger, D. et al., Exp. Neuro. 141: 47-56 (1996); Jeffrey, H. et al., Pharm. Res. 10: 362-68 (1993)), spray drying, and extrusion methods.

Another type of carrier is nanoparticles, which are generally suitable for intravenous administrations. Submicron and nanoparticles are generally made from amphiphilic diblock, triblock, or multiblock copolymers as is known in the art. Polymers useful in forming nanoparticles include, but are limited to, poly(lactic acid) (PLA; see Zambaux et al., J. Control Release 60:179-188 (1999)), poly(lactide-co-glycolide), blends of poly(lactide-co-glycolide) and polycarprolactone, diblock polymer poly(1-leucine-block-1-glutamate), diblock and triblock poly(lactic acid) (PLA) and poly(ethylene oxide) (PEO) (see De Jaeghere, F. et al., Pharm. Dev. Technol.; 5: 473-83 (2000)), acrylates, arylamides, polystyrene, and the like. As described for microparticles, nanoparticles may be non-biodegradable or biodegradable. Nanoparticles may be also be made from poly(alkylcyanoacrylate), for example poly(butylcyanoacrylate), in which the STAT inhibiting agent is absorbed onto the nanoparticles and coated with surfactants (e.g., polysorbate 80). Coating the surface of the particle with a polymer attached to a targeting molecule allows for delivery of the particles to specific cells. Methods for making nanoparticles are similar to those for making microparticles and include, among others, emulsion polymerization in continuous aqueous phase, emulsification-evaporation, solvent displacement, and emulsification-diffusion techniques (see, e.g., Kreuter, J., Nano-particle Preparation and Applications, In Microcapsules and nanoparticles in medicine and pharmacy, (M. Donbrow, ed.), pg. 125-148, CRC Press, Boca Rotan, Fla. (1991); Incorporated by reference).

Hydrogels are also useful in delivering the subject agents into a host. Generally, hydrogels are crosslinked, hydrophilic polymer networks permeable to a wide variety of drug compounds, including proteins and nucleic acids. Hydrogels have the advantage of selective trigger of polymer swelling, which results in controlled release of the entrapped compound. Depending on the composition of the polymer network, swelling and subsequent release may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide), poly(N-isopropylacrylamide); poly(methacrylic acid-g-polyethylene glycol); polyacrylic acid and poly(oxypropylene-co-oxyethylene) glycol; and natural compounds such as chrondroitan sulfate, chitosan, gelatin, or mixtures of synthetic and natural polymers, for example chitosan-poly(ethylene oxide). The polymers are crosslinked reversibly or irreversibly to form gels embedded with the compositions of the present invention (see for example, U.S. Pat. Nos. 6,451,346; 6,410,645; 6,432,440; 6,395,299; 6,361,797; 6,333,194; 6,297,337 Johnson, O. et al., Nature Med. 2: 795 (1996); incorporated by reference in their entirety).

In one embodiment, the gel polymers are acrylic acid polymers, preferably carbomers (e.g., carboxypolymethylene), such as Carbopol (e.g., Carbopol 420-430, 475, 488, 493, 910, 934P, 974P, and the like; Brock et al., Pharmacotherapy 14: 430-437 (1994)), which are non-linear polymers of acrylic acid crosslinked with polyalkenyl polyether. Other types of carbomers include acrylic acids crosslinked with polyfunctional compounds, such as polyallysucrose. In addition to the advantage of hydrating and swelling to a gel, which entraps the subject compounds and limits their release, carbomer gels are mucoadhesive. The polymers adhere to the intestinal mucosal membrane, thus resulting in local delivery of the peptides (see Hutton et al., Clin. Sci. 78: 265-271 (1990); Pullan et al., Gut 34: 676-679 (1993), hereby incorporated by reference). In addition, these polymers have the added advantage of limiting intestinal protease activity. The gel formulations described above are suitable for delivery of nucleic acids as well as proteins. For example, antibody immobilization of virions in a gel using a polyclonal biotinylated IgG specific for the virus particle retains the viral particle in the gel, resulting in localized release at site of implant.

The concentrations of the peptides or nucleic acid encoding therefore will be determined empirically in accordance with conventional procedures for the particular purpose. Generally, for administering STAT inhibitory agents ex vivo or in vivo for therapeutic purposes, the subject formulations are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms of the disorder or disease.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery, composition half-life, and agent efficacy. Stage of the disease to consider include whether the disease is acute or chronic, relapsing or remitting phase, and the progressiveness of the disease. Determining the dosages and times of administration for a therapeutically effective amount are well within the skill of the ordinary person in the art.

For any compounds used in the present invention, therapeutically effective dose is readily determined by methods well known in the art. For example, an initial effective dose can be estimated initially from cell culture assays. An indicator of STAT activity, such as inflammatory response, effectiveness of IL6 signaling, or direct detection of STAT levels may be used. A dose can then be formulated in animal models to generate a circulating concentration or tissue concentration, including that of the IC₅₀ (e.g., dose sufficient to cause 50% inhibition of activity) as determined by the cell culture assays.

In addition, the toxicity and therapeutic efficacy are generally determined by cell culture assays and/or experimental animals, typically by determining a LD₅₀ (lethal dose to 50% of the test population) and ED₅₀ (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein.

Generally, in the case where formulations are administered directly to a host, the present invention provides for a bolus or infusion of the STAT inhibiting protein that will administered in the range of about 0.1-50, more usually from about 1-25 mg/kg body weight of host. The amount will generally be adjusted depending upon the half-life of the peptide, where the half-life will generally be at least one minute, more usually at least about 10 min, desirably in the range of about 10 min to 12 h. Short half-lives are acceptable, so long as efficacy can be achieved with individual dosages, continuous infusion, or repetitive dosages. Formulations for administration may be presented in unit a dosage form, e.g., in ampules, capsules, pills, or in multidose containers or injectables. For treatment using nucleic acids encoding the STAT Inhibiting agents, an effective amount of the pharmaceutical compositions is that which mimics physiological effects observed upon administration of protein formulations to cells.

Dosages in the lower portion of the range and even lower dosages may be employed, where the peptide has an enhanced half-life or is provided as a depot, such as a slow release composition comprising particles, a polymer matrix which maintains the peptide over an extended period of time (e.g., a collagen matrix, carbomer, etc.), use of a pump which continuously infuses the peptide over an extended period of time with a substantially continuous rate, or the like.

In addition to administering the subject peptide compositions directly to a cell culture in vitro, to particular cells ex vivo, or to a mammalian host in vivo, nucleic acid molecules (DNA or RNA) encoding the subject peptides may also be administered thereto, thereby providing an effective source of the subject proteins for the application desired. As described above, nucleic acid molecules encoding the subject peptides may be cloned into any of a number of well known expression plasmids (see Sambrook et al., supra) and/or viral vectors, such as adenoviral or retroviral vectors (see for example, Jacobs et al., J. Virol. 66:2086-2095 (1992), Lowenstein, Bio/Technology 12:1075-1079 (1994) and Berkner, Biotechniques 6:616-624 (1988)), under the transcriptional regulation of control sequences which function to promote expression of the nucleic acid in the appropriate environment. Such nucleic acid-based vehicles may be administered directly to the cells or tissues ex vivo (e.g., ex vivo viral infection of cells for transplant of peptide producing cells) or to a desired site in vivo, e.g. by injection, catheter, orally (e.g., hydrogels), and the like, or, in the case of viral-based vectors, by systemic administration. Tissue specific promoters may optionally be employed, assuring that the peptide of interest is expressed only in a particular tissue or cell type of choice. Methods for recombinantly preparing such nucleic acid-based vehicles are well known in the art, as are techniques for administering nucleic acid-based vehicles for peptide production.

For the purposes of this invention, the methods of administration are chosen depending on the condition being treated, the form of the subject compositions, and the pharmaceutical composition. Administration of the STAT inhibiting proteins and nucleic acids encoding them can be done in a variety of ways, including, but not limited to, cutaneously, subcutaneously, intravenously, orally, topically, transdermally, intraperitoneally, Intramuscularly, nasally, and rectally (e.g., colonic administration). For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by rectal or topical administration to the affected area. Alternatively, oral administration is used in conjunction with intravenous or parenteral injections.

In one embodiment, the method of administration is by oral delivery, in the form of a powder, tablet, pill, or capsule. Pharmaceutical formulations for oral administration may be made by combining one or more STAT inhibiting compositions with suitable excipients, such as sugars (e.g., lactose, sucrose, mannitol, or sorbitol), cellulose (e.g., starch, methyl cellulose, hydroxylmethyl cellulose, carbonxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like. Alternatively, oral formulations of the peptides are in prepared in a suitable diluent. Suitable diluents include various liquid forms (e.g., syrups, slurries, suspensions, etc.) in aqueous diluents such as water, saline, phosphate buffered saline, aqueous ethanol, solutions of sugars (e.g. sucrose, mannitol, or sorbitol), glycerol, aqueous suspensions of gelatin, methyl cellulose, hydroxylmethyl cellulose, cyclodextrins, and the like. In some embodiments, lipohilic solvents are used, including oils, for instance vegetable oils, peanut oil, sesame oil, olive oil, corn oil, safflower oil, soybean oil, etc.); fatty acid esters, such as oleates, triglycerides, etc.; cholesterol derivatives, including cholesterol oleate, cholesterol linoleate, cholesterol myristilate, etc.; liposomes; and the like.

In yet another embodiment, the administration is carried out cutaneously, subcutaneously, intraperitonealy, intramuscularly and intravenously. As discussed above, the compositions to be administered are dissolved or suspended in suitable aqueous medium. Additionally, the pharmaceutical compositions for injection may be prepared in lipophilic solvents, which include, but is not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes, as described above. The compositions may be prepared directly in the lipophilic solvent or preferably, as oil/water emulsions, (see, e.g., Liu, F. et al., Pharm. Res. 12:1060-1064 (1995); Prankerd, R. J., J. Parent. Sci. Tech. 44:139-49 (1990); and U.S. Pat. No. 5,651,991).

The delivery systems also include sustained release or long term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the subject compositions, such as biodegradable polymers described above; pumps, including peristaltic pumps and fluorocarbon propellant pumps; osmotic and mini-osmotic pumps; and the like. Peristaltic pumps deliver a set amount of drug with each activation of the pump, and the reservoir can be refilled, preferably percutaneously through a port. A controller sets the dosage and can also provides a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating a chronic disease condition, especially those manifesting recurring episodes or which are progressive in nature, by delivering the STAT inhibiting agents of the invention via systemic (e.g., intravenous or subcutaneous) or localized doses in a sustained, long term manner.

The present invention also encompasses the therapeutic combinations disclosed herein in the form of a kit or packaged formulation. A kit or packaged formulation as used herein includes one or more dosages of a STAT inhibiting agent in a container holding the dosages together with instructions for simultaneous or sequential administration to an afflicted patient. For example, the package may contain the proteins along with a pharmaceutical carrier combined in the form of a powder for mixing in an aqueous solution, which can be ingested by the afflicted subject. Another example of packaged drug is a preloaded pressure syringe, so that the compositions may be delivered colonically or intravenously. The package or kit includes appropriate instructions, which encompasses diagrams, recordings (e.g., audio, video, compact disc), and computer programs providing directions for use of the combination therapy.

The descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

All publications and patent applications mentioned in this specification are herein incorporated by reference to the same extent as if each individual publication or patent application was specifically and individually indicated to be incorporated by reference.

### EXAMPLES

### Example 1

### Experimental: Materials and Methods

Cells and viruses. Human 2FTGH, 293T, NIH3T3 and 3T3/v-Src, U3A, and U6A cell lines were maintained in Dulbecco's modified Eagle's medium (Gibco-BRL) supplemented with 10% Cosmic calf serum (Hyclone) and 1% penicillin/streptomycin (Gibco-BRL) as described previously (Parisien, J.-P. et al. J. Virol. 76:4190-4198 (2002)). U266 myeloma cells (generous gift of Rich Jove) were grown in RPMI+10% heat inactivated fetal bovine serum. Mumps virus (Enders strain; ATCC# VR-106) was cultivated and titered in Vero cells.

Plasmids, transfection, and reporter gene assays. A cDNA copy of the Mumps virus V gene was amplified by PCR from reverse-transcribed genomic RNA isolated from Vero cells infected with the Enders strain of Mumps virus, using primers that add restriction endonuclease recognition sequences for direct cloning into a mammalian expression plasmid downstream of in-frame N-terminal FLAG or HA epitope tags. Several independently transcribed and amplified cDNAs were sequenced and all were found to differ from the GenBank database sequence entry at three positions influencing the amino acid sequence (position 214: AGC(Ser) instead of AGA(Arg); position 382: GCG(Ala) instead of ACG(Thr); position 433: ACC(Thr) instead of ATC(Ile)). Plasmid pcDNA3-v-Src was the gift of Toru Ouchi.

For luciferase assays in 293T cells, 60 mm dishes were transfected by calcium phosphate method with 2 mg of reporter gene (for IFNα responses, 5×ISRE-luciferase; for IFNγ and IL6 responses, 4×M67 SIE-luciferase), 6 mg of V plasmid, and 0.5 mg of CMV-LacZ. For luciferase assays in other cell lines, cells were transfected using Superfect® reagent (Qiagen) or Lipofectamine® reagent (Gibco) according to the manufacturer's method with a CMV-LacZ plasmid as a control for transfection efficiency, a reporter gene, and either empty vector or cDNA expression plasmids indicated. After 24 h, transfection medium was replaced with fresh medium or medium supplemented with cytokines: 1000 U/ml recombinant human IFNα (Hoffman LaRoche), 1000 U/ml recombinant murine IFNβ for NIH 3T3 cells (PBL Biomedical), or IL6 (400 ng/ml)+soluble IL6 receptor (500 ng/ml) as described (Guschin, D. et al., EMBO J. 14:1421-1429 (1995)). Cells were harvested 6-12 h later in luciferase assay lysis buffer and luciferase activity measured according to manufacturer's protocol (Promega). Values for luciferase activity were normalized to P-galactosidase activity. In all cases, data represent the average values of triplicate samples normalized to co-transfected β-galactosidase activity, expressed as % of control stimulated sample.

Indirect immunofluorescence. For immunofluorescence, cells were grown on permanox chamber slides (Nalgene Nunc) and transfected and stained exactly as described in Rodriguez, J. J. et al., J Virol 76:11476-11483 (2002) but using FLAG antibody (Sigma, 10 mg/ml) STAT3 antibody (Santa Cruz, C-20; 3 mg/ml) and TOTO 3 nuclear stain (Molecular Probes, 48 nM). For infections, cells were infected on chamber slides with 2 pfu/cell of Mumps virus and stained with Mumps NP antibody (Chemicon International, 10 mg/ml). Images were obtained using a Leica TCSSP confocal microscope and representative fields illustrating transfected and untransfected cells in the same field are illustrated, but note that V protein effects on STATs were observed with 100% penetrance, even in cells with relatively low V levels.

Cell extracts, immunoblotting, and immunoprecipitation. For preparation of cell extracts, samples were washed once with ice-cold PBS and subsequently lysed with whole cell extract buffer (WCEB) as described (Parisien, J.-P. et al., supra; Ulane, C. M. and Horvath, C. M. Virology 304:160-166 (2002)). For immunoblotting, proteins were separated and transferred to nitrocellulose, then probed with antibodies and visualized with chemiluminescence (NEN Life Sciences). For immunoprecipitation, lysates were prepared in WCEB and pre-cleared with protein A agarose. Antibody-protein complexes were purified with protein A beads and washed with WCEB. After elution with SDS, proteins were separated by SDS-PAGE, and processed for immunoblotting. Antibodies used: STAT1 (Santa Cruz C20), STAT2 (Santa Cruz, C24), STAT3 (Santa Cruz C20), FLAG (Sigma), ubiquitin (Santa Cruz P4D1), DDB1 (Abcam #9194), or Cullin 4A (Santa Cruz #8557).

Affinity Purification. Affinity purification was carried out exactly as described (Ulane, C. M., supra). Lysates from four 20 transfected 10 cm plates per sample (−50 mg total protein per sample) were treated with 1 mg/ml DNase I for 2 h, and 50 mg/ml ethidium bromide before incubation with anti-FLAG (M2)-agarose beads (200 ml per sample; Sigma) overnight at 4° C. Beads were washed with WCEB, and protein complexes eluted with 100 mg/ml FLAG peptide. Eluates were denatured in SDS loading buffer, separated by SDS-PAGE and processed for either silver staining (Biorad Silver Stain Plus) or immunoblotting.

Annexin staining. Apoptosis was measured by staining with FITC-conjugated annexin V according to manufacturer's procedure (Oncogene Science). Cells were transfected by Lipofectamine (3T3/v-Src) or electroporation (U266) with 16 mg of control vector, SV5 or Mumps V vector, or 5 mg RIP vector and 3 mg of CD14 expression plasmid (generous gifts of Adrian Ting, Mt. Sinai). 48 h later, CD14-positive cells were enriched by magnetic bead selection (Miltenyi biotech), and subject to annexin V staining and flow cytometry on a FacScan (Becton Dickinson). RIP samples were harvested at 24 h post transfection.

### Example 2

### Mumps Virus V Protein Eliminates Both STAT1 and STAT3

A cDNA copy of the Mumps virus V gene was tested in assays for IFN signaling inhibition (see FIG. 7A). Transient expression of Mumps virus V protein was capable of interfering with type I or type II IFN reporter gene assays, consistent with loss of STAT1 signaling. To test the effects of V protein expression on individual STAT proteins, the steady state level of STAT1, STAT2, and STAT3 was examined by indirect immunofluorescence. Epitope tagged V protein was detected with tag-specific antibodies, and STAT1, STAT2, or STAT3 detected In the same cells by double labeling. STAT1 protein level was efficiently reduced in cells expressing Mumps virus V, but no reduction in STAT2 level was observed (FIG. 7B). In addition to its ability to reduce STAT1 levels, the Mumps virus V protein also dramatically reduced the level of STAT3. This novel STAT3-directed activity is a unique property of the Mumps virus V protein that is not shared by SV5 or HPIV2 V proteins (JJR and CMH, unpublished observations). To formally verify that the loss of STAT protein corresponds to an inhibition of STAT protein activation, similar assays were carried out using IFNγ to activate STAT1, IFNα to activate STAT2, or interleukin-6 (IL6) to activate STAT3 (FIG. 7B). As expected from loss of STAT1 or STAT3, no cytokine-induced nuclear translocation was observed in cells expressing Mumps V protein, but adjacent untransfected cells exhibited normal cytokine-dependent nuclear accumulation. Interestingly, the pattern of STAT2 redistribution in response to IFNα was altered, exhibiting a partial nuclear translocation phenotype, consistent with earlier conclusions regarding STAT2 nuclear transfer in the absence of STAT1 (Improta, T. et al., Proc. Natl. Acad. Sci. USA 91: 4776-4780 (1994)).

To evaluate the possibility that expression of Mumps virus V protein outside the context of a virus infection confers the unique STAT3-directed activity, immunofluorescence was used to evaluate STAT protein levels in Mumps virus-infected cells. An antibody that recognizes the Mumps virus nucleocapsid protein (NP) was used to identify infected cells (FIG. 7C). The NP staining was localized to discrete cytoplasmic bodies, a pattern that has been observed for other paramyxoviruses (Fearns, R. et al., J. Gen. Virol. 7: 3525-3539 (1994); Precious, B. et al., J. Virol. 69: 8001-8010 (1995); Randall, R. E. et al., Virology 224: 121-129 (1996)). All cells that stained positive for NP also exhibited loss of STAT1 and STAT3. A significant difference in STAT2 subcellular distribution was also observed in Mumps-infected cells. STAT2 was localized to punctate cytoplasmic bodies that in many instances co-localized with the NP stain. These results demonstrate that in addition to STAT1, Mumps virus V protein has a second host degradation target, STAT3. Mumps virus infection can also alter the subcellular localization of latent STAT2 protein.

### Example 3

### Mumps Virus V Inhibits Cytokine and Oncogene Signaling

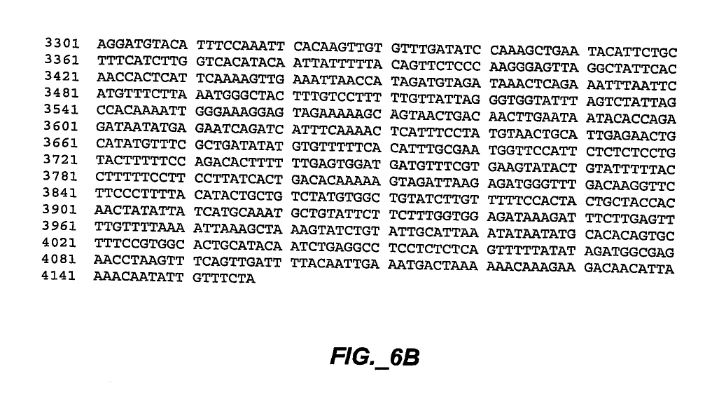

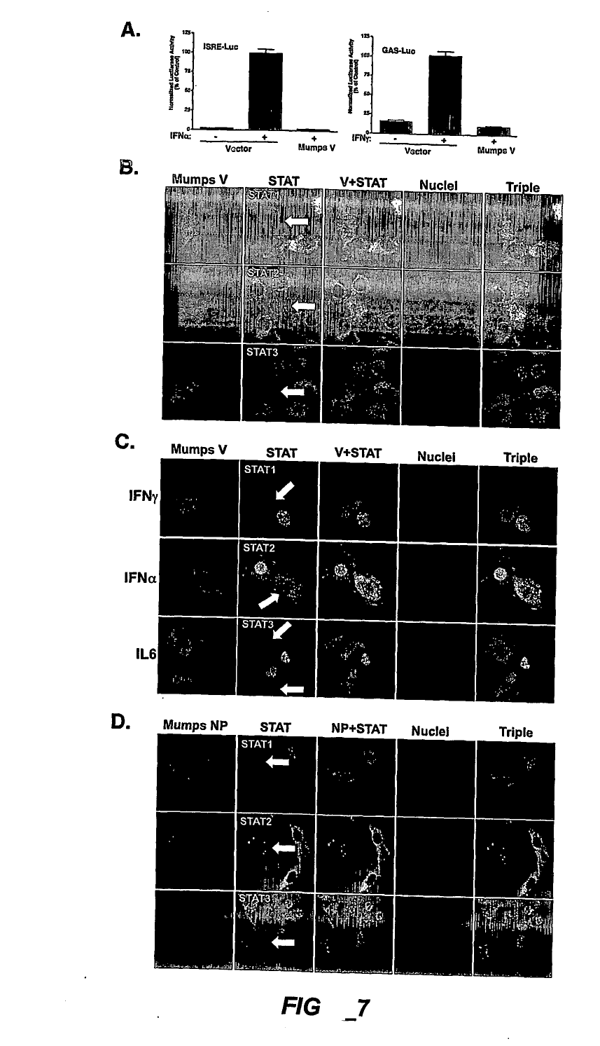

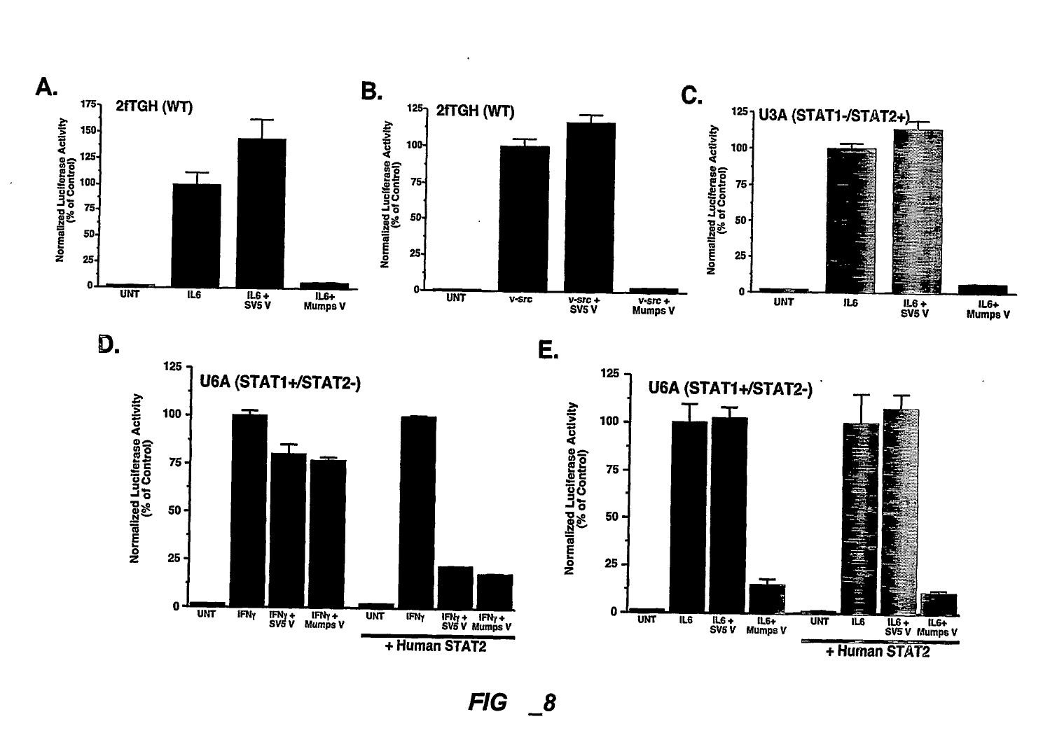

STAT3 activation and transcription factor activity has been well studied for cytokine signaling systems similar to IL6 (Aaronson, D. S. and Horvath, C. M., Science 296:1653-1655 (2002), Heinrich, P. C. et al., Biochem. J. 334 (Pt 2):297-314 (1998)). To test the consequences of V protein-induced STAT3 degradation in a biological context, STAT3-dependent transcription assays were carried out. Treatment with IL6 potently induces reporter gene expression from a STAT3-responsive SIE/GAS-luciferase construct, but expression of Mumps virus V completely prevented reporter gene induction by IL6 (FIG. 8A). No inhibitory effect on IL6 signaling was observed with control SV5 V or HPIV2 V expression, but a notable and reproducible 30-50% increase In IL6 reporter gene activity was observed with SV5 V (FIG. 8A).

To determine the ability of Mumps virus V protein to block STAT3 signaling induced by an intracellular stimulus, v-Src, an oncogenic tyrosine kinase, was used to activate STAT3. Expression of v-Src potently induced the reporter gene, but this induction was specifically abolished by Mumps virus V protein (FIG. 8B). Together, these results demonstrate that the reduction of STAT3 protein level induced by Mumps virus V protein inhibits both extracellular and intracellular STAT3 activating pathways

### Example 4

### Distinct Requirements for Targeting STAT1 and STAT3

Several systems, including IL6, that rely on STAT3 signals can also activate STAT1, and the activated STAT factors recognize the same DNA sequence element (Horvath, C. M. et al., Genes Dev. 9: 984-994 (1995), making STAT1 and STAT3 activity indistinguishable in reporter gene assays. To clearly define the Mumps virus V protein IL6 inhibition as the result of STAT3 and not STAT1 interference, assays were carried out in a STAT1-deficient cell line (U3A cells (McKendry, R. et al., Proc Natl Acad Sci USA 88:11455-11459 (1991), FIG. 8C). In the absence of STAT1, IL6 produced a potent activation of luciferase activity that was completely prevented by Mumps virus V expression, demonstrating that STAT3 and not STAT1 is the Mumps V target responsible for IL6 evasion. This is distinct from HPIV2-induced STAT2 targeting, which requires STAT1 in an interdependent process. These data suggest a unique mechanism is used by Mumps virus for STAT3 interference that is STAT1-independent.

Analogous to HPIV2, SV5 only degrades STAT1 In the presence of cellular STAT2 (Parisien, J.-P. et al., supra). To investigate a role for STAT2 in Mumps virus V protein targeting, assays were carried out in STAT2-deficient cells (U6A (Leung, S. et al., Mol Cell Biol 15:1312-1317 (1995)). IFNg-STAT1 signaling was robust In the STAT2-deficient U6A cells, and significant interference by either SV5 or Mumps virus V proteins was only observed when STAT2 was provided by co-transfection (FIG. 8D). This finding demonstrates that, like SV5, Mumps virus V protein requires STAT2 in order to antagonize STAT1 signaling. IL6-STAT3 signaling, which is robust in U6A cells, was unaffected by SV5 V either in the presence or in the absence of expressed STAT2 (FIG. 8E). In contrast, expression of Mumps virus V protein potently prevents IL6-STAT3 signaling in both the absence and presence of STAT2 (FIG. 8E). These data indicate conspicuously different STAT2 requirements in Mumps-dependent STAT1 or STAT3 degradation.

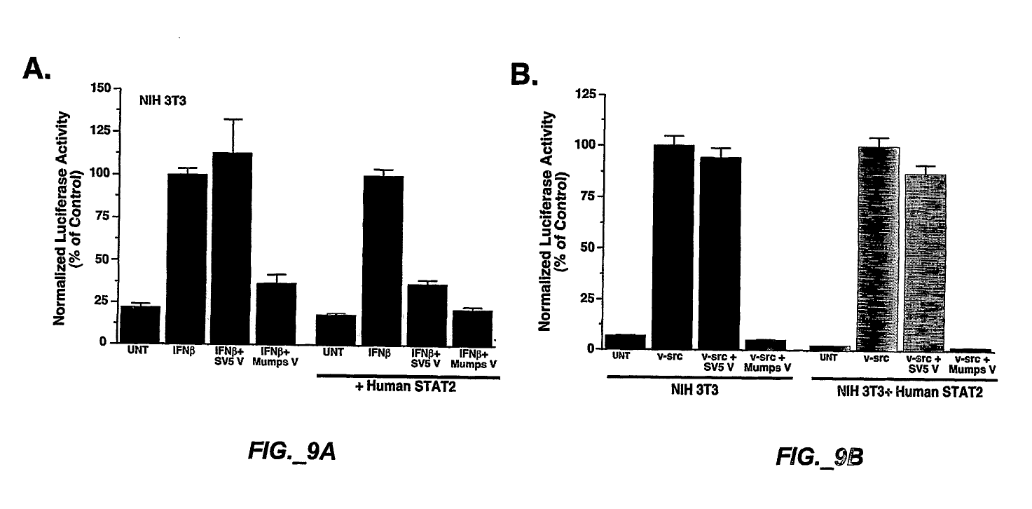

For SV5, the critical role of STAT2 In STAT1 degradation is underscored by the discovery that differences between human and murine STAT2 orthologues provide a barrier to SV5-induced STAT1 degradation in the mouse system (Parisien, J. P. et al., J Virol. 76:6435-6441. (2002)). To test the Mumps V protein for species-restricted STAT2 requirement, mouse NIH 3T3 fibroblast cell lines were used for transcription interference assays. While SV5 requires human STAT2, Mumps virus V is able to antagonize IFNβ signaling in the murine system, independent of human STAT2 expression (FIG. 9A). These results indicate that while STAT1 targeting by Mumps V absolutely requires STAT2, Mumps V activity is not restricted by murine and human STAT2 differences.

The ability of Mumps virus V to antagonize murine STAT3-dependent signaling was also tested using v-Src as the inducer in either NIH3T3 cells or a derivative with stable expression of human STAT2 (FIG. 9B). Mumps virus V protein disrupted v-Src signaling In the mouse cells irrespective of human STAT2 expression. Together, the differences in STAT2-dependence suggest Mumps virus V protein utilizes distinct targeting mechanisms for STAT1 or STAT3 degradation.

### Example 5

### Mumps Virus V Assembles a STAT Targeting Complex

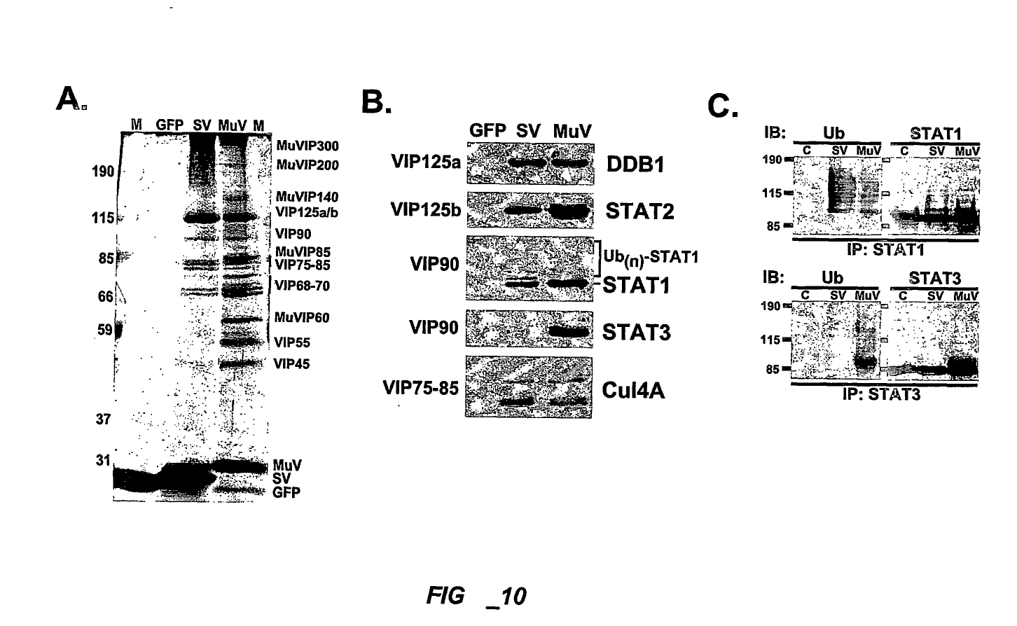

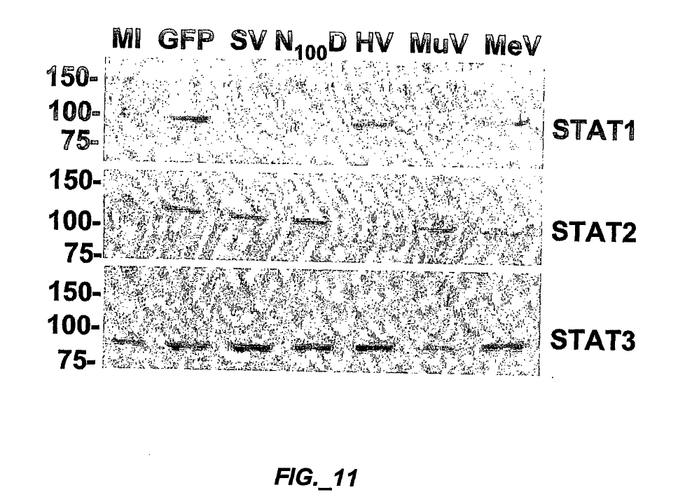

STAT protein targeting by SV5 and HPIV2 requires a multi-subunit VDC Ub ligase complex that is comprised of cellular components including STAT1 and STAT2, DDB1, and CuI4a (Ulane, C. M. and Horvath, C. M., Virology 304:160-166 (2002)). To determine if the Mumps virus V protein assembles a similar STAT degradation complex, FLAG-tagged Mumps V and SV5 V were subject to affinity chromatography. Analysis of the affinity purified material by SDS PAGE and silver staining revealed a number of V-interacting protein (VIP) species that ranged in apparent molecular weight between 40 and 300 kDa that co-purified with either Mumps V or SV5 V protein but not a FLAG-tagged GFP control (FIG. 10A). The cellular proteins associated with the two viral proteins comprise a pattern that is overall very similar and likely represents a core degradation complex with virus-specific differences providing the basis for differential STAT targeting specificity. Significant Mumps V-specific polypeptides are apparent at ˜85 and ˜60 kDa, but less abundant Mumps-specific bands are observed at ˜300, 200 and 140 kDa.

Immunoblotting with specific antisera (FIG. 10B) reveals that both V protein affinity preparations contain STAT1 and STAT2, consistent with the genetic data described above. The STAT1 immunoblot revealed a laddering pattern for both SV5 and Mumps V proteins, consistent with V-mediated STAT1 modification by polyubiquitylation. In addition, both viruses co-purified cellular DDB1 and CuI4A, two proteins with demonstrated roles in SV5 V-dependent STAT1 targeting. Immunoblotting with STAT3 antiserum reveals that only the Mumps virus V protein affinity preparation contains co-purified STAT3. Importantly, none of these partner proteins was detected in the GFP control. To test the ability of the V proteins to induce specific STAT protein polyubiquitylation, transfection conditions that produced V protein levels sub-optimal for complete degradation were used to capture the unstable polyubiquitylated STAT intermediates. Immunoprecipitation of the target STAT protein followed by immunoblot with Ub-specific antiserum revealed that both SV5 and Mumps V protein induced polyubiquitylation of STAT1 (FIG. 10C, top panels). In contrast, only Mumps virus V protein induced polyubiquitylation of STAT3 (FIG. 10C, bottom panels). These results are in agreement with the concept that the Mumps virus V protein induces the formation of a STAT1-and-STAT2-containing VDC Ub ligase complex that resembles the SV5 VDC, but Mumps virus V protein forms additional superimposed associations with STAT3 through a related but mechanistically distinct multi-protein Ub ligase.

### Example 6

### Oncolytic Activity of Mumps Virus V Protein

The constitutively activated STAT3 found in many human cancers often functions in a survival role for tumor maintenance, and inhibition of STAT3 has been demonstrated to induce apoptosis in tumor cells. Malignant transformation of cultured murine fibroblasts by v-Src requires functional STAT3 signaling (Bromberg, J. et al., Cell. Biol. 18:2553-2558 (1998)). Similarly, growth and survival of human myeloma tumor cells depends on IL6-mediated STAT3 signaling. The human U266 myeloma cell line possesses an autocrine IL6 self-stimulatory loop that produces constitutively activated STAT3. In these cancer cells, disruption of STAT3 signaling induces spontaneous apoptosis (Catlett-Falcone, R. et al., Immunity 10:105-115 (1999)). To test for oncolytic activity, U266 or 3T3/V-Src cells were transfected with plasmids encoding V proteins along with an expression vector encoding CD14, a monocyte cell surface protein used as a marker for selecting transfected cells. As a positive control, parallel U266 samples were transfected with an expression vector for RIP, a pro-apoptotic death domain protein. Enriched CD14-positive cell fractions were isolated with magnetic beads, and equal numbers of cells stained with annexin V (Table 1). Slight increases in the percentage of annexin-positive apoptotic cells were observed after expression of control vector or SV5 V protein (11.1% and 15.8% respectively for U266; 29.7% and 25.2% for 3T3/V-Src), but expression of Mumps virus V protein substantially increased the number of annexin-positive cells (34.9% for U266; 57.4% for 3T3N-Src). The apoptosis induced by Mumps virus V protein in U266 cells was similar to that induced by the positive control, RIP (40.6%). Expression of Mumps V also correlated with a reduced soft agar plating efficiency for the 3T3N-Src cells (not shown). The induced apoptosis correlates with STAT3 degradation and v-Src or IL6 suppression, and suggests a therapeutic potential for the Mumps virus V protein.

TABLE I
Mumps virus V protein induces apoptosis in cancer cells.

| Cell Line | Transfection¹ | Annexin Positive² |
|---|---|---|
| U266 | Vector | 11.1 |
| | SV5V | 15.8 |
| | Mumps V | 34.9 |
| | RIP | 40.6 |
| 3T3/v-Src | Vector | 29.7 |
| | SV5V | 25.2 |
| | Mumps V | 57.4 |

¹Cells were co-transfected with expression vector for CD14.
²Percent of 1 × 10⁵ CD14-positive cells that stain with FITC-conjugated Annexin V measured by flow cytometry.

Without being bound by theory, the evidence provided above suggests that the Mumps V protein induces assembly of a protein complex similar to but distinct from the VDC ubiquitin ligase complex assembled by SV5, and catalyzes polyubiquitylation of both STAT1 and STAT3. As a consequence, STAT3-dependent transcription is disengaged in cells expressing Mumps virus V protein. Comparison of the STAT1 and STAT3 targeting requirements indicate separate mechanisms are used for each STAT. We demonstrate that the STAT3 interference is equally effective for suppressing transcription initiated by either cytokine (IL6) or oncogene (v-Src) signals. Moreover, Mumps virus V protein oncolytic potential is revealed by Mumps V-dependent apoptosis induced in human multiple myeloma cells and transformed murine fibroblasts. STAT3 interference by Mumps virus or the Mumps virus V protein represents a previously unrecognized STAT3 targeting agent with therapeutic potential.

### Example 7

### Delivery of MUMPS V Protein by Lentivirus Vectors

Lentivirus expression vector construction is based on vector pLenti as described in Mitta, B. et al., Nucleic Acids Res. 30(21):e113 (2002). The pLenti viral vectors are self-inactivating vectors derived from HIV-1 based SIN pNL-EGFPΔU3. Coding regions of various genes, including the DNA coding for the Mumps V protein, were cloned into the vector via standard recombinant techniques. Human fibrosarcoma 2fTGH cells were infected with 1×10⁵ colony forming units of replication-deficient recombinant virus, and whole cell lysates prepared 48 hours post infection. Lysates were processed for immunoblotting and detected with antiserum to STAT1, STAT2, and STAT3. Immunoblots were developed via standard chemiluminescent detection technique. Recombinant viruses expressing the following proteins were used: GFP, green fluorescent protein; SV, SV5 V protein; N100D, variant of SV5 V protein; HV, HPIV2 V protein; MuV; Mumps virus V protein; and MeV, measles virus V protein.

### Example 8

### Downregulation of STAT 3 Activity In Vivo

Inhibition of STAT-3 activity by delivery of Mumps V protein In vivo. To downregulate the activity of STAT3 in an animal model, any of the delivery methods mentioned could be used to express a mumps V protein-encoding cDNA. One way to accomplish such a delivery would be to construct a recombinant viral vector, possibly a lentivirus based system, to produce a replication-incompetent viral vector. Another way is to use the mumps virus itself. The recombinant virus could be applied by direct inoculation of target tissues. To directly evaluate the ability of such a delivery to inhibt STAT3 activity, several methods can be used. The total level of STAT3 in the targeted tissue can be analyzed by immunoblot. The specific reduction in STAT protein levels can be determined by processing tissue specimens for immunohistochemistry with STAT-specific antisera. In addition, target tissues can be analyzed for STAT target gene expression. For example, well known STAT3 targets are BclXI, c-myc, and cyclin D1. Reverse transcription PCR (RT-PCR) assays with specific oligonucleotide primers will be used to test that these genes are inhibited by loss of STAT3 activity.

### Example 9

### Inhibition of Tumor Cell Growth In Vivo

Inhibition of tumor growth or cancer by delivery or expression of Mumps V protein. For a murine tumor model, host mice are first implanted with syngenic tumor cells by subdermal injection to form a visible tumor. After measuring the diameter, the tumors are directly injected with a solution containing the recombinant virus (or other delivery vehicle) expressing mumps V protein or a control virus expressing an inert protein (e.g. green fluorescent protein). In some animals, control or inoculated tumors are harvested for biochemical and histological analysis. These samples are subject to RNA and protein extraction to facilitate analysis of STATs and their target genes including examination of STAT target genes by RTPCR methods as mentioned above, or protein levels by immunoblotting or in situ protein analysis immunohistochemistry. In other animals, the tumor size is measured and monitored for several days prior to sacrifice. Given the importance of STAT3 as a tumor survival factor, the tumors inoculated with control preparations are expected to continue to grow larger during the time course of the experiment, while the test group inoculated with mumps virus or mumps V vectors are expected to undergo apoptosis, or cell death, leading to tumor stasis or shrinkage.

Similar methodology could be used to evaluate the effects of reducing STAT3 in inflammatory responses. For example, a murine model system for chronic inflammation could be similarly targeted with Mumps V expression vectors, and the STAT3 expression levels examined by immunohistochemistry, and correlated with reduced inflammation by standard histological techniques.

What is claimed is:

1. A method of modulating STAT3 mediated signaling in a cell, comprising:
contacting the cell with a STAT inhibitor, wherein said inhibitor comprises a protein having at least about 80% identity with the amino acid sequence of SEQ ID NO: 1, and wherein said protein decreases the level of STAT3.

2. The method according to claim 1 wherein said protein comprises the amino acid sequence of SEQ ID NO: 1.

3. The method according to claim 1, wherein said protein comprises a fusion protein.

4. The method according to claim 1 wherein said protein comprises a peptide fragment of SEQ ID NO: 1.

5. The method according to claim 1, wherein said protein is expressed from a nucleic acid encoding said protein, wherein said nucleic acid comprises a nucleic acid sequence having at least about 80% identity to the nucleic acid sequence of SEQ ID NO: 2.

6. The method according to claim 5, wherein said nucleic acid comprises the nucleic acid sequence of SEQ ID NO:2.

7. The method according to claim 6, wherein said nucleic acid is part of a viral vector.

8. The method according to claim 7, wherein said viral vector comprises a lentiviral vector.

9. The method according to claim 1, wherein said contacting is in vitro.

10. The method according to claim 1, wherein said contacting is in vivo.

11. The method according to claim 1, wherein said cell is a tumor cell.

12. The method according to claim 1, wherein said cell is a lymphocyte.

13. The method according to claim 12, wherein said lymphocyte is a T-cell.

14. The method according to claim 1, wherein the cell interacts with a cytokine capable of activating STAT3.

15. The method according to claim 14, wherein said cytokine is growth hormone, IL6, IL10, or G-CSF.

16. A method for inhibiting growth of a tumor cell, comprising:
contacting the tumor cell with a composition comprising a protein having at least about 80% identity with the amino acid sequence of SEQ ID NO: 1, wherein said protein has STAT inhibiting activity.

17. The method according to claim 16, wherein said protein comprises the amino acid sequence of SEQ ID NO: 1.

18. The method according to claim 16, wherein said protein comprises a fusion protein.

19. The method according to claim 17, wherein said protein comprises a peptide fragment of SEQ ID NO: 1.

20. The method according to claim 16, wherein said protein is expressed from a nucleic acid encoding said protein, wherein said nucleic acid comprises a nucleic acid sequence having at least about 80% identity with the nucleic acid sequence of SEQ ID NO: 2.

21. The method according to claim 20, wherein said nucleic acid comprises the nucleic acid sequence of SEQ ID NO: 2.

22. The method according to claim 21, wherein said nucleic acid is part of a viral vector.

23. The method according to claim 22, wherein said viral vector is a lentiviral vector.

24. The method according to claim 16, wherein said contacting is in vitro.

25. The method according to claim 16, wherein said contacting is in vivo.

26. The method according to claim 16, wherein said STAT inhibiting activity is for STAT1 or STAT3, or variants thereof.

27. The method according to claim 16, wherein said tumor cell is associated with elevated STAT3 activity.

28. The method according to claim 27, wherein said tumor cell is multiple myeloma, leukemia; lymphomas, cutaneous T-cell lymphoma, Hodgkin's disease; and solid tumors.

29. The method according to claim 16, wherein said protein is used in combination with a chemotherapeutic agent.

30. A method for inhibiting an inflammatory reaction, comprising:
administering to a subject an effective amount of a composition comprising a protein having at least about 80% identity to the amino acid sequence of SEQ ID NO: 1 in an amount sufficient to inhibit said inflammatory reaction, wherein said protein has STAT inhibiting activity.

31. The method according to claim 30, wherein said protein comprises the amino acid sequence of SEQ ID NO: 1

32. The method according to claim 30, wherein said protein comprises a fusion protein.

33. The method according to claim 30 wherein said protein comprises a peptide fragment of SEQ ID NO:1.

34. The method according to claim 30, wherein said protein is expressed from a nucleic acid encoding said protein, wherein said nucleic acid comprises a nucleic acid sequence having at least about 80% identity to the nucleic acid sequence of SEQ ID NO:2.

35. The method according to claim 34, wherein said nucleic acid comprises the nucleic acid sequence of SEQ ID NO:2.

36. The method according to claim 30, wherein said STAT inhibiting activity is for STAT1 or STAT3, or variants thereof.

37. The method according to claim 30, wherein said inflammatory reaction is associated with elevated STAT3 activity.

38. The method according to claim 37, wherein said inflammatory reaction is associated with Crohn's disease, inflammatory bowel disease, multiple sclerosis, ischemia, stroke, traumatic brain injury, spinal injury, rheumatoid arthritis, and atherosclerosis.

39. A method for treating an autoimmune disease, comprising:
administering to a subject an effective amount of a composition comprising a protein having at least about 80% identity with the amino acid sequence of SEQ ID NO:1, wherein said protein has STAT inhibiting activity.

40. The method according to claim 39, wherein said protein comprises the amino acid sequence of SEQ ID NO:1.

41. The method according to claim 39, wherein said protein comprises a fusion protein.

42. The method according to claim 39 wherein said protein comprises a peptide fragment of SEQ ID NO:1.

43. The method according to claim 39, wherein said protein is expressed from a nucleic acid encoding said protein, wherein said nucleic acid has a nucleic acid sequence of at least about 80% identity to the nucleic acid sequence of SEQ ID NO:2.

44. The method according to claim 43, said nucleic acid comprises SEQ ID NO:1.

45. The method according to claim 39, wherein said STAT inhibiting activity is that of STAT1 or STAT3, or variants thereof.

46. The method according to claim 39, wherein said autoimmune disease is associated with elevated levels of STAT3 activity.

47. The method according to claim 46, wherein said autoimmune disease is insulin dependent diabetes mellitus, systemic lupus erythremetosus, or psoriasis.


SEQUENCE LISTING (13 sequences)

SEQ ID NO: 1 - protein, 224 aa, Mumps virus
MDQFIKQDETGDLIETGMNVANHFLSAPIQGTNSLSKASIIPGVAPVLIGNPEQKNIQHPTASHQGSKSKGSGSGVRSIIVPPSEAGNGGTQIPEPLFAQTGQGGIVTTVYQDPTIQPTGSYRSVELAKIGKERMINRFVEKPRTSTPVTEFKRGAGSGCSRPDNPRGGHRREWSLSWVQGEVRVFEWCNPICSPITAAARFHSCKCGNCPAKCDQCERDYGPP

SEQ ID NO: 2 - DNA, 675 bp, Mumps virus
atggatcaatttataaaacaggatgagactggtgatttaattgagacaggaatgaatgttgcaaatcatttcctatccgcccccattcagggaaccaactcgctgagcaaggcctcaatcatccctggcgttgcacctgtactcattggcaatccagagcaaaagaacattcagcaccctaccgcatcacatcagggatccaagtcaaagggcagcggctcaggggtcaggtccatcatagtcccaccctccgaagcaggcaatggagggactcagattcctgagcccctttttgcacaaacaggacagggtggtatagtcaccacagtttatcaggatccaactatccaaccaacaggttcataccgaagtgtggaattggcgaagatcggaaaagagagaatgattaatcgatttgttgagaaacctagaacctcaacgccggtgacagaatttaagaggggggccgggagcggctgctcaaggccagacaatccaagaggagggcatagacgggaatggagcctcagctgggtccaaggagaggtccgggtctttgagtggtgcaaccctatatgctcacctatcactgccgcagcaagattccactcctgcaaatgtgggaattgccccgcaaagtgcgatcagtgcgaacgagattatggacctccttag

SEQ ID NO: 3 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 4 - DNA, 3455 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaaaaaaaaaaa

SEQ ID NO: 5 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 6 - DNA, 4157 bp, Homo sapiens
agcggggcggggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatgcacacagtgctttccgtggcactgcatacaatctgaggcctcctctctcagtttttatatagatggcgagaacctaagtttcagttgattttacaattgaaatgactaaaaaacaaagaagacaacattaaaacaatattgtttcta

SEQ ID NO: 7 - protein, 9 aa, Human immunodeficiency virus
RKKRRQRRR

SEQ ID NO: 8 - protein, 32 aa, Artificial
  Synthetic
XXXXXCXXXCXXXXXXXXXXXXHXXXHXXXXX

SEQ ID NO: 9 - protein, 33 aa, Artificial
  Synthetic
FQCEECXXXXXXXXXXXXXXXXXXXXHIRSHTG

SEQ ID NO: 10 - protein, 30 aa, Artificial
  Synthetic
CXXCXXXXXXXXXXXXXXXXXXXXHXXXXC

SEQ ID NO: 11 - protein, 33 aa, Artificial
  Synthetic
VKCFNCXXXXXXXXXXXXXXXXXXXXHTARNCR

SEQ ID NO: 12 - protein, 34 aa, Artificial
  Synthetic
MNPNCARCGXXXXXXXXXXXXXXXXXXXXHKACF

SEQ ID NO: 13 - protein, 21 aa, Apis mellifera
MKFLVDVALVFMVVYISYIYA